(19) United States
(12) Patent Application Publication
(10) Pub. No.: US 2024/0182526 A1
(43) Pub. Date: Jun. 6, 2024

(54) COMPOSITIONS AND METHODS FOR MODULTING INFLAMMATORY AND DEGENERATIVE DISORDER

(71) Applicant: University of Southern California, Los Angeles, CA (US)

(72) Inventors: Denis Evseenko, Tarzana, CA (US); Ruzanna Shkhyan, Los Angeles, CA (US)

(21) Appl. No.: 18/285,227

(22) PCT Filed: Mar. 30, 2022
(86) PCT No.: PCT/US2022/022559

§ 371 Date: Sep. 29, 2023

Related U.S. Application Data
(60) Provisional application No. 63/168,901, filed on Mar. 31, 2021.

(51) Int. Cl. C07K 7/64; A01K 67/0275; A61K 31/4439; A61K 38/12; A61P 19/02; A61P 29/00; C07K 14/705; C07K 16/28

(52) U.S. Cl. CPC C07K 7/64; A01K 67/0275; A61K 31/4439; A61K 38/12; A61P 19/02; A61P 29/00; C07K 14/705; C07K 16/2866; A01K 2227/105

(57) ABSTRACT

The disclosure provides compounds and method useful for modulating gp130 biological activity. The disclosure also provides methods and compositions for treating disease and disorders associated with gp130 activity, particularly those associated with inflammation.

### CROSS REFERENCE TO RELATED APPLICATIONS

This application claims priority under 35 U.S.C. § 119 from Provisional Application Ser. No. 63/168,901, filed Mar. 31, 2021, the disclosures of which are incorporated herein by reference.

### TECHNICAL FIELD

The disclosure provides compounds and method useful for modulating gp130 biological activity. The disclosure also provides methods and compositions for treating disease and disorders associated with gp130 activity, particularly those associated with inflammation.

### INCORPORATION BY REFERENCE OF SEQUENCE LISTING

Accompanying this filing is a Sequence Listing entitled, “Sequence-Listing_ST25.txt, created on Mar. 30, 2022 and having 33,421 bytes of data, machine formatted on IBM-PC, MS-Windows operating system. The sequence listing is hereby incorporated herein by reference in its entirety for all purposes.

### BACKGROUND

Osteoarthritis (OA) is a degenerative disease of joints, characterized by progressive loss of cartilage which causes stiffness, swelling and pain. Nearly 10% of the world's population suffers from OA, making it the most common form of arthritis and one of the most common pathological conditions. In the United States, there are over 27 million people affected by OA, with this number projected to climb steeply due to a rapidly aging population and increases in obesity rates. Each year, over $185 billion is spent to treat OA globally, establishing this disease as a major burden on global health and economics.

Currently, there are no disease modifying agents available on the market for the treatment of osteoarthritis. Treatment modalities are focused on lifestyle modifications, pain management and improving joint viscosity, with the overall goal to delay joint replacement surgery. Initial therapies for those with mild osteoarthritis include weight loss, physical therapy and pain management using over-the-counter non-steroidal anti-inflammatory drugs (NSAIDs). As the condition progresses to a moderate stage, opioid-based pain control is introduced, while continuing physical therapy and other exercises. If the disease progresses to a severe stage, intra-articular injections, such as hyaluronic acid, are used to increase joint viscosity. Finally, if none of the previous treatments are able to mitigate pain, total joint replacement surgery is considered. There are over 1 million knee and hip replacements performed each year in the U.S. at a cost of over $50 billion.

### SUMMARY

The disclosure provides an isolated peptide comprising, consisting essentially of or consisting of 4-50 amino acids and containing the sequence QQ(PY)F, wherein the peptide interacts with c-SRC. In one embodiment, the peptide is cyclized with a reagent, thereby forming a cyclic peptide. In a further embodiment, the reagent is a thiol-reactive reagent. In yet a further embodiment, the thiol-reactive reagent is selected from the group consisting of: 1,2-bis(bromomethyl)benzene, 1,4-bis(bromomethyl)benzene, 2,6-bis(bromomethyl)pyridine, substituted bis(bromomethyl)benzenes and (E)-1,4-dibromobut-2-ene. In still a further embodiment, the thiol-reactive reagent is 1,3-bis(bromomethyl)benzene. In another embodiment, the peptide comprises a cell penetrating peptide (CPP) sequence. In still another embodiment, the peptide comprises one or more non-naturally occurring amino acids. In yet another embodiment, the peptide comprises one or more D-amino acids. In another embodiment, the peptide comprises a sequence set forth in Table 1 or a derivative thereof. In one embodiment, the peptide comprises the sequence QQ(PY)F.

The disclosure also provides a pharmaceutical composition comprising any of the peptides/peptidomimetics described herein and above.

The disclosure also provides a method for the treatment of a gp130 associated disease or disorder in a subject comprising administering to said subject in need thereof of a therapeutically effective amount of a peptide or a pharmaceutical composition of the disclosure. In one embodiment, the gp130 disease or disorder is an inflammatory disease or disorder or cell proliferative disease or disorder. In a further embodiment, the inflammatory disease or disorder or cell proliferative disease or disorder is selected from the group consisting of stroke, heart disease, cartilage degeneration, hair-loss, wound healing, arthritis, neurodegenerative disorders, aging, psoriasis, rosacea, lupus, rheumatoid arthritis, inflammatory bowel disease, fibrosis and cancer.

The disclosure also provides a method of modulating IL-6 mediated inflammatory cascade in a cell comprising contacting the cell with a peptide or pharmaceutical composition comprising said peptide of the disclosure. In one embodiment, the cell is a chondrocyte.

The disclosure also provides a method of treating an acute or chronic inflammatory state comprising contact a subject with a peptide or pharmaceutical composition comprising said peptide of the disclosure.

The disclosure also provides a method of decreasing an activated inflammatory pathway in a cell comprising contact the cell with a peptide or pharmaceutical composition comprising said peptide of the disclosure.

The disclosure also provide a method of inhibiting the production or induction of pro-inflammatory genes, cytokines or mediators comprising contacting a cell or subject with a peptide or pharmaceutical composition comprising said peptide of the disclosure.

The disclosure also provides a method of inhibiting the production or induction of extracellular matrix degrading enzymes comprising contacting a cell or subject with a peptide or pharmaceutical composition comprising said peptide of the disclosure.

The disclosure also provides a polyclonal antibody that specifically binds to an epitope comprising or corresponding to pY814 of SEQ ID NO:26 (human gp130). In one embodiment, the polyclonal antibody is labelled.

The disclosure also provides a method of diagnosing an inflammatory disease or disorder comprising contacting a cell comprising a gp130 with a polyclonal antibody of the disclosure and determining the binding of the polyclonal antibody to the gp130, wherein the binding is indicative of the inflammatory disease or disorder.

The disclosure also provides a non-human transgenic animal expressing a gp130 variant, wherein the gp130 variant comprises a substitution corresponding to the tyrosine residue at position 814 of SEQ ID NO:26 (human gp130) with a phenylalanine. In one embodiment, the animal is a mouse. In another or further embodiment, the mouse comprises a gp130 variant having a sequence of SEQ ID NO:27, wherein the gp130 variant comprises an F812 substitution.

The disclosure also provides a method of screening for substances for treating an inflammatory disease or disorder, pain, cancer or fibrosis in association with IL-6 family cytokine receptor malfunction, said method comprises using a non-human transgenic animal of the disclosure.

The best characterized facet of gp130 signaling is its ability to promote or suppress inflammation resulting in tissue regeneration or pathology. This suggests that the divergence in outcomes downstream of gp130 are differentially regulated, which imparts the need for identification of a novel specific modality within this network that can be manipulated to initiate beneficial outcomes. Gp130 regulates signaling cascades via recruitment of proteins on its various residues; however, some of these signaling residues have not been well-characterized. The disclosure demonstrates that a signaling Y814 residue within gp130 module serves as a major cellular stress sensor that is responsible for inducing pro-inflammatory and pro-fibrotic outcomes along with SRC kinase recruitment. Instigation and maintenance of inflammation during disease pathogenesis is carried out by pro-inflammatory mediators that are tightly regulated. The data presented here validates that Y814 is responsible for regulating most of the genes that are involved in inflammation and fibrosis including IL-6 cytokines, proteases, cellular adhesion molecules that are critical in leukocyte recirculation, COX2 and others. Since excess inflammation is a foremost culprit in the dysregulation of normal wound healing, targeting Y814 may potentially orchestrate efficacious tissue healing processes and microenvironment.

The disclosure demonstrates that gp130 Y814 serves as a recruitment site for SRC kinase. In addition, mutation in Y814 also downregulates MAPK (ERK 1/2) signaling, which is a central pathway controlling cellular processes associated with fibrogenesis, including growth, proliferation, and survival. It is unclear whether SRC and MAPK/ERK crosstalk on Y814, but is conceivable as SRC is known to activate ERK signaling in multiple pathologies. Conversely, other pathways downstream of gp130 such as STAT3 and AKT remained minimally affected.

An RNA seq analysis demonstrated herein shows that ablation of Y814 in vivo can dramatically reduce expression of multiple pro-inflammatory and pro-fibrotic genes in response to OSM (e.g. COX2, IL-11, NF-kB, etc.), in the skin wound healing and joint destabilization injury models, Y814 transgenic mice (F814) demonstrated a regenerative and anti-fibrotic healing potential while averting degenerative processes. The disclosure demonstrates that activation of this pro-inflammatory and pro-fibrotic stress sensor is a factor limiting regenerative capacity of adult mammals.

In addition, the disclosure demonstrates that an optimized RCGD423 analog R805 prevents Y814 activation in response to IL-6 cytokines dramatically reducing the appearance of OA in rat and canine models.

The disclosure provides a specific, biologically essential Y814 cellular stress sensor within gp130 module serves as a novel therapeutic target for drug development holding a considerable promise from a translational standpoint. Pharmacological manipulation of specific gp130 modules that enhance intrinsic regenerative capacity of tissues and resistance to degenerative changes can offer safe symptomatic treatments and alternatives to the conventional treatment options and could revolutionize the contemporary approach for a broad range of conditions. Therapeutic approaches designed towards enhancement of regenerative abilities could also result in substantial lifespan improvements.

The disclosure provides an isolated peptide of 4-50 amino acids that contains the sequence QQ(pY)F. In one embodiment, the peptide is cyclized with a reagent, thereby forming a cyclic peptide. In a further embodiment, the reagent is a thiol-reactive reagent. In still a further embodiment, the thiol-reactive reagent is selected from the group consisting of: 1,2-bis(bromomethyl)benzene, 1,4-bis(bromomethyl)benzene, 2,6-bis(bromomethyl)pyridine, substituted bis(bromomethyl)benzenes and (E)-1,4-dibromobut-2-ene. In one embodiment, the thiol-reactive reagent is 1,3-bis(bromomethyl)benzene. In one embodiment, the peptide further comprises a cell penetrating peptide (CPP or PTD) sequence. In one embodiment, the peptide comprises one or more non-naturally occurring amino acids. In one embodiment, the peptide comprises one or more D-amino acids. In one embodiment, the peptide comprises a sequence set forth in Table 1 and containing QQ(pY)F.

The disclosure also provides pharmaceutical composition comprising the peptide of the disclosure and a pharmaceutically acceptable carrier. In one embodiment, the pharmaceutical composition further comprises a compound Of

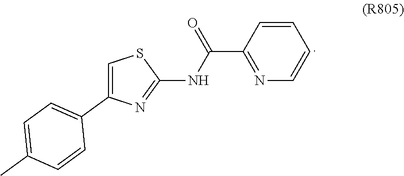 [SMILES: Cc1ccc(-c2csc(NC(=O)c3ccccn3)n2)cc1]

The disclosure also provides a method for the treatment of a gp130 associated disease or disorder in a subject comprising administering to said subject in need thereof of a therapeutically effective amount of a peptide or a pharmaceutical composition of the disclosure. In one embodiment, the gp130 disease or disorder is an inflammatory disease or disorder or cell proliferative disease or disorder. In one embodiment, the inflammatory disease or disorder or cell proliferative disease or disorder is selected from the group consisting of stroke, heart disease, cartilage degeneration, hair-loss, wound healing, arthritis, neurodegenerative disorders, aging, psoriasis, rosacea, lupus, rheumatoid arthritis, inflammatory bowel disease, fibrosis and cancer.

The disclosure also provides a method of modulating IL-6 mediated inflammatory cascade in a cell comprising contacting the cell with a peptide or a pharmaceutical composition of the disclosure. In one embodiment, the cell is a chondrocyte.

The disclosure also provides a method of treating an acute or chronic inflammatory state comprising contact a subject with a peptide of the disclosure.

The disclosure also provides a method of decreasing an activated inflammatory pathway in a cell comprising contact the cell with a peptide or a pharmaceutical composition of the disclosure.

The disclosure also provides a method of inhibiting the production or induction of pro-inflammatory genes, cytokines or mediators comprising contacting a cell or subject with a peptide or a pharmaceutical composition of the disclosure.

The disclosure also provides a method of inhibiting the production or induction of extracellular matrix degrading enzymes comprising contacting a cell or subject with a peptide or a pharmaceutical composition of the disclosure.

The disclosure also provides a polyclonal antibody that specifically binds to an epitope comprising or corresponding to pY814 of SEQ ID NO:26. In one embodiment, the polyclonal antibody is labelled.

The disclosure also provides a method of diagnosing an inflammatory disease or disorder comprising contacting a cell comprising a gp130 with a polyclonal antibody of the disclosure and determining the binding of the polyclonal antibody to gp130, wherein the binding is indicative of the inflammatory disease or disorder.

The disclosure also provides a non-human transgenic animal expressing a gp130 variant or homolog, wherein the gp130 variant or homolog comprises a substitution corresponding to the tyrosine residue at position 814 of SEQ ID NO:26 with a phenylalanine and wherein the transgenic animal exhibits reduced fibrosis and improved tissue regeneration compared to a wild-type animal. In one embodiment, the animal is a mouse. In another embodiment, the mouse comprises a gp130 variant having a sequence of SEQ ID NO:27, wherein the gp130 variant comprises an F812 substitution.

The disclosure also provides a method of screening for substances for treating an inflammatory disease or disorder, pain, cancer or fibrosis in association with IL-6 family cytokine receptor malfunction, said method comprises using a transgenic animal of the disclosure. In one embodiment, the inflammatory disease or disorder comprises arthritis.

The disclosure also provides a transgenic mouse model comprising a knockout of gp130 Y814.

### DESCRIPTION OF DRAWINGS

FIG. 1A-D shows genetically modified Y814 (F814) murine model. (A) CRISPR gRNA was designed by PNA Bio and the Y814 mutant mouse (F814) was generated by the USC transgenic mouse core. (B) WT and F814 mouse knees. (C) Levels of pSRC and pNF-κB p65 in WT and F814 mouse spleen stimulated with or without OSM for 24 hours. (D) Levels of protein complex formation between gp130 and pSRC in WT and F814 mouse spleen stimulated with or without OSM for 24 hours.

FIG. 2A-C shows molecular and functional effects of a recently identified chemical modulator of gp130. (A) Effects of CX-011 in canine model of posttraumatic OA. 24 dogs were randomized in four groups and treated with different doses of CX-011. (A) depicts OARSI scoring of joint sections by a blinded observer. Lower score means less progression of degenerative joint disease. (B) depict Colorado Pain Scores for each group over the course of the experiment as assessed by blinded observers, respectively. Surgeries and analysis were performed by Dr. Mark Hurtig (Guelph, Canada). (C) Protein levels of gp130 pY814 in pig articular chondrocytes stimulated with or without OSM for 24 hours in presence or absence of CX-011. Histone H3 was used as a loading control.

FIG. 3 shows a schematic of the study.

FIG. 4A-C shows OSM activity induces pro-inflammatory signaling and matrix catabolism that can be blocked by SRC inhibition. (A) Transcription of catabolic genes was determined via qPCR in pig articular chondrocytes treated with or without OSM, SRC inhibitor (SU6656) or both. (B) Protein levels of pNF-κB p⁶⁵ in pig articular chondrocytes stimulated with or without OSM and SRC inhibitor (SU6656) for 24 hours. (C) Protein levels of pNF-κB p⁶⁵ and pSRC in ATDC5 cells transfected with the indicated variants of plasmids stimulated with or without OSM for 24 hours.

FIG. 5 shows transcription of catabolic and anabolic genes was determined via qPCR in ATDC5 cells treated with SRC inhibitor alone, transfected with indicated variants of plasmids and stimulated with or without OSM.

FIG. 6 shows Y814 activation is cytokine specific. Protein levels of gp130 pY814 in WT mouse spleen, F814 mouse spleen and fat, and ATDC5 cells stimulated with or without OSM or LIF for 24 hours.

FIG. 7A-B shows F814 mutation reduces degenerative changes in cartilage cells. (A) Protein levels of pTAK1 in mouse WT and F814 spleen stimulated with or without OSM for 24 hours. Histone H3 was used as a loading control. (B) Mouse WT and F814 knee cartilage explants were incubated with or without OSM for 72 hours; levels of cleaved aggrecan and collagen epitopes in the supernatant are normalized to the wet weight of the explant.

FIG. 8A-B shows P2A inhibits SRC activity. (A) Levels of complex formation between gp130 and pSRC in pig articular chondrocytes stimulated with or without OSM in presence or absence of P2A for 24 hours. Total gp130 was used as a loading control. (B) Protein levels of pSRC and pNF-kB p65 in pig articular chondrocytes stimulated with or without OSM in presence or absence of P2A for 24 hours. Total gp130 was used as a loading control.

FIG. 9 shows P2A promotes biosynthesis and inhibits transcription of matrix degrading genes in OA chondrocytes. Transcription of anabolic and catabolic genes was determined via qPCR in human adult OA articular chondrocytes treated with or without P2A.

FIG. 10 shows inhibition of matrix loss by P2A is dose dependent. Pig articular cartilage explants were stimulated with or without OSM and treated with or without P2A at difference doses for 72 hours; levels of cleaved aggrecan epitope in the supernatant are normalized to the wet weight of the explant.

FIG. 11 shows Y814 is upregulated in 2 mouse models of arthritis. Endogenous levels of gp130 pY814 and pSRC in mouse synovium (C57 Black strain) from medial meniscal (MM) tear and collagen-induced arthritis (CIA) models. Synovial membrane was harvested at week 4 after MM surgery and at day 7 after induction of CIA model. Representative blots (n=3). Control represents the protein levels of pY814 in synovial tissue of non-manipulated mice. Histone H3 was used as a loading control.

FIG. 12 are schematics depicting mechanisms of gp130 inflammatory cascade. Cytokine oncostatin M (OSM) activates residue Y814 of gp130. Phosphorylation of this residue results in recruitment and activation of c-SRC kinase and subsequent activation of the master transcription regulator of inflammation NF-κB. LIF is a different cytokine from the IL6 family; LIF has no ability to activate Y814 and instead triggers a different signaling program downstream of gp130.

FIG. 13 is a schematic depicting the mode of action of the P2A peptide. The P2A peptide blocks the interaction between gp130 Y814 and c-SRC and inhibits OSM-induced pro-inflammatory cascade.

FIG. 14A-B provides Table 1 and composition data of Formula I. (A) Table 1. Peptide candidates tested (SEQ ID Nos:61-71, respectively). (B) Structure and properties of P2A.

FIG. 15 shows that Peptide 5 inhibits endogenous SRC and NF-kB protein expression. Protein levels of phosphorylated c-SRC (pSRC) and NF-kB p65 in pig articular chondrocytes treated with respective peptides for 24 hours. Total H3 was used as a loading control. H3—histone H3.

FIG. 16 shows Peptide 5 inhibits SRC and NF-kB protein expression. Protein levels of pSRC and pNF-kB p65 in human synovium stimulated with or without OSM in presence or absence of respective peptides for 24 hours. Histone 3 was used as a loading control.

FIG. 17 shows that inhibition of matrix loss by P2A is dose dependent. Pig articular cartilage explants were stimulated with or without OSM and treated with or without P2A at different doses for 72 hours; levels of cleaved aggrecan epitope in the supernatant are normalized to the wet weight of the explant.

FIG. 18A-D shows Y814 residue regulates SRC signaling downstream of gp130. A. qPCR results from pig articular chondrocytes stimulated with OSM and treated with or without SRC inhibitor (SU6656) for 48 hours. Horizontal lines with bars show the mean±SD. n=3. B. Protein expression of pSRC in ATDC5 cells after transfection (72 hours) with the modified and WT gp130 plasmids stimulated with OSM for 24 hours. Horizontal lines with bars show the mean±SD. n=3. C. qPCR results from ATDC5 cells treated with SRC inhibitor (SU6656) alone, or transfected with indicated variants of plasmids and stimulated with or without OSM for 48 hours. Horizontal lines with bars show the mean±SD. n=3. D. Protein expression of gp130 pY814 in untreated fetal (17 weeks) and adult articular chondrocytes. Horizontal lines with bars show the mean±SD. n=3.

FIG. 19A-G shows that genetic knockout of a novel gp130 modality in vivo results in downregulation of pro-degenerative and pro-fibrotic signaling cascades. A. Protein expression of pSRC in pig articular chondrocytes stimulated with IL-6 cytokines. Horizontal lines with bars show the mean±SD. n=3. B. Schematic of modified amino acids within gp130 812-827 domain. Protein expression of pSRC in Ba/F3 cells after transfection with the modified or WT gp130 plasmids stimulated with IL-6 for 24 hours. Horizontal lines with bars show the mean±SD. n=3. C. CRISPR gRNA was designed by PNA Bio. Y814 mutant mouse (F814) was generated by the USC transgenic mouse core. Mice genotyping was performed by GeneWiz. Representative images of 4-month-old males shown. n=4. (SEQ ID NO:59 and 60). D. Protein expression of gp130 pY814 in wild type (WT) and F814 mouse splenocytes treated with and without OSM and LIF for 24 hours. Horizontal lines with bars show the mean±SD. n=3. E. Distribution of differentially expressed genes in MA plot of wild type (WT) and F814 mouse periarticular stromal cells. MA plots (log 2fold change vs log 2mean expression) for WT-OSM vs WT and Y814-OSM vs Y814 mouse periarticular stromal cells. Each dot represents a gene. Red dots represent upregulated genes i.e. log 2fold-change >1 and p-value<0.05. Blue dots represent downregulated genes i.e. log 2fold change <−1 and p-value 0.05. Genes that do not qualify this threshold are indicated by grey dots. F. Levels of protein complex formation between gp130 and pSRC in wild type and F814 mouse splenocytes stimulated with or without OSM for 24 hours. Horizontal lines with bars show the mean±SD. n=3. G. Protein expression of pSRC in wild type and F814 mouse splenocytes stimulated with or without OSM for 24 hours. Horizontal lines with bars show the mean±SD. n=3.

FIG. 20A-B shows F814 mouse exhibits no noticeable morphological differences in major organs. A. H&E staining for indicated wild type (WT) and F814 mouse organs. Magnification=20×. n=3. B. Graphs indicate the average body weight (grams) of 4-month-old males. Representative images are shown. Horizontal lines with bars show the mean±SD. n=3.

FIG. 21 shows that the F814 mouse does not express gp130 Y814. Protein expression of gp130 pY814 in wild type (WT) and F814 mouse splenocytes treated with and without OSM and LIF for 4 hours. Horizontal lines with bars show the mean±SD. n=3.

FIG. 22 shows OA pathway analysis. RNA-seq results demonstrated that deletion of Y814 can dramatically reduce expression of pro-inflammatory genes in response to OSM with Ingenuity Macrophage RA pathway being one of the most affected by this mutation. Murine primary fibroblasts were stimulated with OSM for 24 hours.

FIG. 23 shows wild type and F814 mice have different modes of regulation downstream of gp130. Protein expression of indicated proteins in wild type (WT) and F814 mouse primary periarticular fibroblasts stimulated with or without OSM or LIF for 4 and 24 hours. Horizontal lines with bars show the mean±SD. n=3.

FIG. 24 shows ablation of Y814 inhibits OSM-induced collagenase and aggrecanase activity. Wild type and F814 mouse knee articular cartilage explants were incubated with or without OSM for 72 hours followed by a neoepitope assay. Horizontal lines with bars show the mean±SD. n=3.

FIG. 25A-D shows that Gp130 Y814-deficient mouse demonstrates increased resistance to degenerative joint disease and enhanced skin regeneration after injury. A. Histological staining and quantitative assessment of cartilage degradation and changes in joint morphology of wild type (WT) and F814 mouse knee joints 6 weeks after destabilization of the medial meniscus surgery. Safranin O delineates proteoglycans (pink). OARSI scoring system was used to quantify the extent of cartilage damage. Scale bars represent 100 μm. Horizontal lines with bars show the mean±SD. n=6. B. Wound-induced hair neogenesis in F814 mice is enhanced. WT and F814 mouse post-wound day (PWD) 21 wound sections after wound excision. Representative images are shown. Horizontal lines with bars show the mean±SD. n=5. C. Re-clustering of clusters annotated as macrophages from WT and F814 skin wounds PWD 14. Dot plots depict gene expression in each macrophage cluster. Clusters are named based on highly significant biomarkers and gene expression profiles. The contribution of each sample to each cluster is shown as a stacked bar graph. Dot sizes are proportional to the percentage of cells in each clutster expressing the indicated gene. DCs=dendritic cells. D. Re-clustering of clusters annotated as fibroblasts from WT and 4814 skin wounds post-wound day (PWD) 14. Dot plots depicting gene expression in each fibroblast cluster. The contribution of each sample to each cluster is shown as a stacked bar graph.

FIG. 26 shows that F814 mouse exhibits reduced synovitis after surgically induced osteoarthritis. Quantitative assessment of synovial lining cell layer, inflammatory infiltrate and density of cells in wild type (WT) and F814 in mouse knee joints 6 weeks after destabilization of the medial meniscus surgery. n=9.

FIG. 27 shows that F814 mouse exhibits enhanced wound-induced hair neogenesis. Wild type (WT) and F814 mouse post-wound day (PWD) 21 wound sections after wound excision. Representative images are shown. n=6.

FIG. 28 shows that F814 mouse cells are resistant to OSM-induced collagen deposition. Picrosirius Red staining of periarticular subcutaneous adipose fibroblasts in wild type (WT) and F814 mouse. Representative images are shown. n=3.

FIG. 29 shows scRNA-sequencing of large wounds from F814 and wild type (WT) mice. UMAP and k-means clustering of equal numbers of cells isolated at post-wound day (PWD) 14 from the indicated genotype; the number of cells in each cluster from each genotype is displayed in the stacked bar graph. Presumptive identities of each cluster are displayed based on biomarker genes significantly enriched; see also Supp. Table X. Cells were harvested from wounds pooled from 2-3 animals of each genotype and sorted as DAPI and Ter119 negative then subjected to the 10× Genomics workflow.

FIG. 30A-E shows a SRC-targeting peptide QQpYF prevents physical interaction of gp130 and SRC and ameliorates degenerative outcomes. A. Levels of complex formation between gp130 and pSRC in pig articular chondrocytes stimulated with or without OSM in presence or absence of control scrambled peptide or peptide QQpYF for 24 hours. Horizontal lines with bars show the mean±SD. n=3. B. Protein levels of pSRC in pig articular chondrocytes stimulated with or without OSM in presence or absence of control scrambled peptide or peptide QQpYF for 24 hours. Horizontal lines with bars show the mean±SD. n=3. C. Computational analysis using GOLD software predicted a high affinity binding of peptide QQpYF to the regulatory site of SRC (c-SRC). c-SRC as visualized by crystallography. The structure of the indicated c-SRC domains is shown in ribbon diagram representation (left) as well as with electrostatic potential (blue, positive charge; red, negative charge; white, neutral) mapped onto the molecular surface (right). Peptide QQpYF is shown in stick representation. D. Transcription of genes was determined via qPCR in human adult OA articular chondrocytes treated with or without peptide QQpYF for 48 hours. Horizontal lines with bars show the mean±SD. n=3. E. Pig knee cartilage explants were stimulated with or without OSM and treated with or without peptide QQpYF at indicated doses for 72 hours followed by a neoepitope assay. Levels of cleaved ACAN and COL2 neoepitopes in the supernatant were quantified with respect to the wet weight of the explant. Horizontal lines with bars show the mean±SD. n=3.

FIG. 31A-B shows that peptide QQpYF inhibits activation of SRC downstream of gp130. A. Levels of complex formation between gp130 and pSRC in pig articular chondrocytes stimulated with or without OSM in presence or absence of control scrambled peptide or peptide QQpYF for 4 hours. Horizontal lines with bars show the mean±SD. n=3. B. Protein levels of pSRC in pig articular chondrocytes stimulated with or without OSM in presence or absence of control scrambled peptide or peptide QQpYF for 4 hours. Horizontal lines with bars show the mean±SD. n=3.

FIG. 32 shows that STAT3 upregulation is high in diseased tissue. Endogenous protein expression of indicated proteins in healthy and osteoarthritic chondrocytes. Horizontal lines with bars show the mean±SD. n=9.

FIG. 33 shows that R805 does not activate MYC signaling. Protein levels of c-MYC in pig articular chondrocytes stimulated with or without RCGD423 or R805. Horizontal lines with bars show the mean±SD. n=3.

FIG. 34A-G shows Gp130 Y814-targeting small molecule promotes regeneration of tissues after injury. A. Protein expression of gp130 pY814 in pig articular chondrocytes treated with or without OSM and R805 for 24 hours. Horizontal lines with bars show the mean±SD. n=3. B. Gross morphology and Masson's trichrome staining of bearded dragon treated with vehicle control and R805 14 days post-amputation. Dashed lines mark amputation planes. n=10. sc=spinal cord; we=wound epithelium; ve=vertebra. C. Gross morphology of bearded dragon co-treated with vehicle control or R805 and PBS or clodronate liposomes 14 days post-amputation. Dashed lines mark amputation planes. n=10. D. Mouse vehicle-treated or R805-treated post-wound day (PWD) 21 wound sections. Representative images are shown. n=8. E. Hair follicle (n=3) and fiber length (n=8) in R805-treated mouse PWD 14 and untreated control wounds. Horizontal lines with bars show the mean±SD. F. Re-clustering of clusters annotated as macrophages from skin wounds of vehicle-treated or R805-treated mice PWD 14. Dot pious depict gene expression in each macrophage cluster. The contribution of each sample to each cluster is shown as a stacked b r graph. Dot: sizes are, proportional to the percentage of cells in each cluster expressing the indicated gene. DCs=dendritic cells. G. Re-clustering of clusters annotated as fibroblasts from skin wounds of vehicle-treated or R805-treated mice PWD 14. Dot plots depict gene expression in each fibroblast cluster. The contribution of each sample to each cluster is shown as a stacked bar graph.

FIG. 35 shows that R805 prevents pro-inflammatory signaling in diseased tissue. Protein expression of indicated proteins in osteoarthritic chondrocytes treated with or without R805 for 4 and 24 hours. Protein levels were quantified with respect to Histone H3. Horizontal lines with bars show the mean±SD. n=9.

FIG. 36 shows that R805 inhibits heterodimerization of gp130 with IL-6 family of cytokines' alpha receptors. Receptor-competition assay. Adult human articular chondrocytes were treated with or without indicated IL-6 family of cytokines and different doses of R805 for 4 hours. Co-IP measuring protein expression was performed after transfection with gp130 FLAG plasmid (72 hours) followed by western blot with respective receptor antibodies. Horizontal lines with bars show the mean±SD. n=3.

FIG. 37 shows that R805 inhibits heterodimerization of gp130 with IL-6 family of cytokines' alpha receptors. Receptor-competition assay. Adult human articular chondrocytes were treated with or without indicated IL-6 family of cytokines and different doses of R805 for 24 hours. Co-IP measuring protein expression was performed after transfection with gp130 FLAG plasmid (72 hours) followed by western blot with respective receptor antibodies. Horizontal lines with bars show the mean±SD. n=3.

FIG. 38A-C shows that R805 exhibits a protective effect on OSM-induced cartilage degeneration in vitro. A. Protein expression of gp130 pY814 in pig articular chondrocytes treated with or without OSM and R805 for 4 hours. Horizontal lines with bars show the mean±SD. n=3. B. qPCR results from pig articular chondrocytes cells treated with OSM with or without R805 for 48 hours. Horizontal lines with bars show the mean±SD. n=3. C. Pig cartilage knee explants were incubated with or without OSM for 72 hours with or without R805 at indicated concentrations followed by a neoepitope assay. Horizontal lines with bars show the mean±SD. n=3.

FIG. 39 shows that R805 prevents pro-inflammatory signaling in OSM-stimulated pig articular chondrocytes. Protein expression of pSRC in pig articular chondrocytes treated with or without OSM and R805 for 4 and 24 hours. Horizontal lines with bars show the mean±SD. n=3.

FIG. 40 show that R805 does not affect pro-regenerative signaling downstream of gp130. Protein expression of indicated proteins in pig articular chondrocytes treated with or without LIF and R805 for 4 and 24 hours. Horizontal lines with bars show the mean±SD. n=3.

FIG. 41 shows that R805 prevents SRC binding to gp130. Levels of complex formation between gp130 and pSRC in pig articular chondrocytes stimulated with or without OSM in presence or absence of R805 for 24 hours. Horizontal lines with bars show the mean±SD. n=3.

FIG. 42A-B shows that R805 treatment enhances lizard tail regeneration in a macrophage-dependent manner. A. EdU and CTSK staining of bearded dragon (Pogona vitticeps) tails treated with vehicle control or R805 14 days post-amputation. n=10. po, periosteum; sc, spinal cord; we, wound epithelium; ve, vertebra. B. EdU and CTSK staining of bearded dragon (Pogona vitticeps) tails treated with vehicle control or R805 14 days post-amputation. n=10. po, periosteum; sc, spinal cord; we, wound epithelium; ve, vertebra.

FIG. 43 shows R805-treated mouse exhibits enhanced wound-induced hair neogenesis. Wild type (WT) and R805-treated mouse post-wound day (PWD) 21 wound sections after wound excision. Representative images are shown. n=6.

FIG. 44 provides data on scRNA-sequencing of large wounds from R805-treated and control mice. UMAP and k-means clustering of equal numbers of cells isolated at post wound day (PWD) 14 from the indicated treatment; the number of cells in each cluster from each treatment is displayed in the stacked bar graph. Presumptive identities of each cluster are displayed based on biomarker genes significantly enriched. Cells were harvested from wounds pooled from 2-3 animals of each treatment and sorted as DAPI and Ter119 negative then subjected to the 10× Genomics workflow.

FIG. 45A-B demonstrates that Intra-articular injection of R805 reduces cartilage degeneration following medial meniscal tear surgery in rats. A. Intra-articular injection of R805 reduces cartilage degeneration following medial meniscal tear surgery in rats; note increased retention of Safranin O+ cartilage. Safranin O staining is proportional to the proteoglycan content in the cartilage tissue. Horizontal lines with bars show the mean±SD. Representative images are shown. n=8. B. Automated indentation and biomechanical thickness mapping of ipsilateral femoral condyle of Sham, Vehicle and R805-treated groups. The instantaneous modulus map shows stiff regions (high instantaneous modulus) of the condyle, which suggests bone exposure. R805 maintained thickness and reduced stiffness in rat cartilage. Representative images are shown. n=8. Mpa=megapascals.

FIG. 46A-B shows intra-articular injection of R805 in dog joint. A. R805 IC50 concentrations of R805 in the Beagle joint during a 90-day pharmacokinetic study. 1 μg intra-articular injection of R805 was maintained for 3 weeks. The compound was not detected in systemic circulation. n=3. B. Schematic depicting the large animal model of osteoarthritis experiment. Intra-articular injections of R805 or vehicle occurred at post-operative weeks 4 and 11. n=7.

FIG. 47A-F shows R805 demonstrates disease modifying effects in small and large animal models of osteoarthritis. A. OARSI scoring of canine joint sections. Horizontal lines with bars show the mean±SEM. n=5-6. B. Synovial inflammation, as gauged by fibrillations and immune infiltration, is reduced by mid and high doses of R805 in canine joint. Representative images are shown. Horizontal lines with bars show the mean±SEM. n=5-6. C. Assessment of canine bone mineral density (BMD) in the medial compartment of the operated stifle by microCT revealed a consistent absence of sclerosis in R805 10 and 1 μg groups. In contrast, BMD in the contralateral, non-operated medial compartment increased with decreasing dose of R805. Horizontal lines with bars show the mean±SEM. n=5-6. D. Canine knee joint shape change from baseline as determined by serial CT imaging showed dose-dependent reduction in ectopic bone formation (red). Horizontal lines with bars show the mean±SEM. n=5-6. E. Canine Colorado Pain Scores were measured daily and totaled for each group starting at after the first intra-articular injection. Linear regression analysis of daily scores is presented; p value. n=5-6. F. Linear regression analysis of lameness scores of R805-treated (10 ug) and vehicle-treated canine group. n=5-6.

FIG. 48 shows results of scRNA-sequencing of synoviocytes from R805-treated and control dogs. UMAP and k-means clustering of equal numbers of cells isolated at time of sacrifice from the indicated treatment; the number of cells in each cluster from each treatment is displayed in the stacked bar graph. Presumptive identities of each cluster are displayed based on biomarker genes significantly enriched; see also Supp. Table X. Cells were harvested from synovial membranes pooled from 2-3 animals of each treatment, subjected to RBC lysis, sorted as DAPI negative and then incorporated into the 10× Genomics workflow.

FIG. 49A-C shows that R805 promotes an anti-inflammatory, anti-fibrotic milieu in an injured joint. A. Re-clustering of clusters annotated as macrophages from synovial joint of R805-treated and vehicle-treated canine group. Clusters are named based on highly significant biomarkers and gene expression profiles. The contribution of each sample to each cluster is shown as a stacked bar graph. B. Dot plots depicting gene expression in each macrophage cluster from synovial joint of R805-treated and vehicle-treated canine group. Dot sizes are proportional to the percentage of cells in each cluster expressing the indicated gene. C. Re-clustering of clusters annotated as fibroblasts from synovial joint of R805-treated and vehicle-treated canine group. Cells are colored by their sample of origin. The dashed oval indicates clusters of fibroblasts dominated by R805-treated cells, while the solid oval denotes a cluster mainly derived from control cells. Select gene ontology terms over-represented in genes significantly enriched when comparing cells in the solid vs. dashed ovals.

### DETAILED DESCRIPTION

As used herein and in the appended claims, the singular forms “a,” “an,” and “the” include plural referents unless the context clearly dictates otherwise. Thus, for example, reference to “a chondrocyte” includes a plurality of such chondrocytes and reference to “an antagonist” includes reference to one or more antagonists or equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood by one of ordinary skill in the art to which this disclosure belongs. Although many methods and reagents similar to or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

Regeneration refers to a type of healing where a compensatory new growth completely restores original tissue architecture and function following damage. Regenerative capacity of an organ is influenced by regulatory networks orchestrated by local and systemic immune responses after an insult. During prenatal development, mammals have an extraordinary ability to regenerate tissue; unfortunately, this capability declines with age as adult injuries are usually not regenerated, but repaired. Some organisms, however, retain the ability to regenerate tissue throughout adult life, which continuously regrow their limbs or organs after amputation or wounding, respectively. Thus, it is of fundamental importance to decipher molecular mechanisms controlling repair versus regenerative responses after wounding.

In high mammals, loss of regenerative ability in the adult can be due to inability to activate this reprogramming process, which may be in turn suppressed by inappropriate level of inflammatory response. Controlled signaling components of the local microenvironment, such as inflammatory reactions initiated by pro-inflammatory cytokines contribute to successful tissue regeneration. During regeneration, inflammation often precedes the actual repair of the lesion. Previous studies across a variety of injury models suggest that a dampened inflammatory response can facilitate regeneration. The IL-6 family of cytokines (e.g., IL-6, IL-11, OSM, LIF, etc.), which signal through glycoprotein 130 (gp130), are pleiotropic key players in inflammatory responses following injury that are capable of promoting both, regeneration and pathogenesis, depending on the perseverance of inflammatory signal. It has been reported that IL-6 cytokines including IL-6 and IL-11 play an important role during limb regeneration after injury in axolotl. The regenerative capacity of IL-6-family cytokines are also seen in adult mammals in various organs and tissues. However, aberrant prolonged activation of IL-6 cytokines is implicated in the pathogenesis of various fibrotic pathologies and complex chronic diseases such as osteoarthritis, where progressive inflammatory and degenerative changes in articular cartilage are also accompanied by the excessive fibroplasia and collagen production in synovial tissue and subchondral bone.

The disclosure shows, for the first time, that a signaling tyrosine 814 (Y814) within gp130 receptor serves as a major cellular stress sensor. Using genetic experiments, in vivo inhibition of this signaling module improves regenerative outcomes in several tissues. Furthermore, a small molecule, R805, that can inhibit gp130 Y814 outputs further provides a major disease modifying agent in a large animal model of OA. R805 has the general structure:

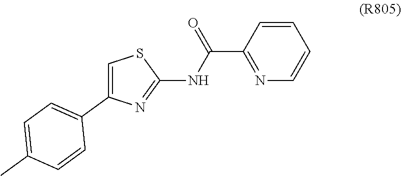 [SMILES: Cc1ccc(-c2csc(NC(=O)c3ccccn3)n2)cc1]

As described further herein, an RNA seq analysis demonstrates that ablation of Y814 in vivo can dramatically reduce expression of multiple pro-inflammatory and pro-fibrotic genes in response to OSM (e.g., Cox2, Il-11, Nf-kb, etc.). The disclosure demonstrates that in a skin wound healing and joint destabilization injury models, Y814 transgenic mice having an F814 demonstrated a regenerative, anti-fibrotic healing potential while averting degenerative processes, respectively. Based on this data, activation of this pro-inflammatory and pro-fibrotic stress sensor limits regenerative capacity. Genetic knockout of Y814 resulted in a decrease of inflammatory gene signature as visualized by the anti-inflammatory macrophage and non-pathological fibroblast subpopulations in the damaged skin tissue, and this improvement in the microenvironment may contribute to the observed enhanced regeneration.

In addition, the disclosure shows that an optimized RCGD 423 analog, R805, prevents Y814 activation in response to IL-6 cytokines conveying specific mode of regulation without hindering other pro-regenerative pathways and dramatically reduces the appearance of OA in rat and canine models. Similar to the ablation of the Y814 in mouse, R805 decreases the pathological milieu in mouse skin wound and diseased canine joint. In combination with the lizard tail regeneration, all these findings suggest that both Y814 and, consequently, R805 may modulate beneficial outcomes via macrophage polarization towards an anti-inflammatory phenotype.

Thus, the disclosure identifies a specific, biologically essential Y814 cellular stress sensor within gp130 receptor, which serves as a novel therapeutic target for drug development and holds considerable promise from a translational standpoint. Pharmacological manipulation of specific gp130 modules that enhance intrinsic regenerative capacity of tissues and resistance to degenerative changes can offer safe symptomatic treatments and alternatives to the conventional treatment options that could revolutionize the contemporary approach for a broad range of conditions. Therapeutic approaches designed towards enhancement of regenerative abilities could also result in substantial lifespan improvements.

The pathogenesis of osteoarthritis (OA) often begins from an injury to articular cartilage, which establishes chronic, low-grade inflammation mediated by interleukin-6/glycoprotein 130 (IL-6/gp130) and other factors that promote matrix degradation over time and eventual destruction of cartilage. IL-6 signaling through IL-6R/gp130 suppresses chondrocyte proliferation, promotes mineralization in articular cartilage, downregulation of matrix proteins and increases expression of matrix-degrading proteases. Moreover, blockade of IL-6 in vivo in mouse models of OA has been shown to be chondroprotective. Importantly, higher serum levels of IL-6 have been correlated with the development of OA in humans, and a monoclonal antibody against IL-6R is currently in Phase III clinical trials for the treatment of hand OA (NCT02477059). Signaling downstream of IL-6/gp130 is mediated by multiple pathways, including signal transducer and activator of transcription 3 (STAT3). STAT3 has been demonstrated to have pleiotropic effects during chondrogenesis and in articular chondrocytes. During chondrogenic differentiation of multipotent mesenchymal stem cells, IL-6/STAT3 signaling promotes chondrocyte commitment and matrix production. Similarly, loss of STAT3 during limb formation results in increased hypertrophy, premature ossification and decreases in expression of the master regulator of chondrocyte identity SOX9. In contrast, in adult articular chondrocytes inhibition of STAT3 downstream of IL-6 is chondroprotective, reducing the severity of OA-like pathology in a mouse model. Together, these data indicate that IL-6/STAT3 signaling can drive matrix loss and development of OA in vivo in both mouse models and humans.

There are two major types of inflammation, acute and chronic. Acute inflammation is rapid and temporary induced by leukocytes in injured tissues. Chronic inflammation is persistent inflammation characterized by tissue injury; the chronic response increases damage to tissues, resulting in the onset of various diseases, such as OA, rheumatoid arthritis (RA), arteriosclerosis, fibrosis and more. IL-6 cytokines (IL-6, IL-11, IL-27, LIF, OSM, CNTF, CT-1, CLCF1) play a pathological role in chronic inflammation and immune response leading to matrix catabolism and tissue destruction. The cellular effects of chronic inflammation have recently been recognized as major contributors to aging and age-associated disease such as OA, leading to the coining of the term inflammaging. The cytokines share a ubiquitously expressed signal transducing receptor, gp130. Aberrant activation of gp130 signaling promotes non-specific tissue damage, resulting in various autoimmune diseases, tissue destruction, fibrosis, thrombocytosis, and may promote malignant transformation. In acute experimental colitis, systemic deletion of gp130 attenuated the mucosal inflammatory infiltrate. In the synovial fluid of knees, IL-6 is thought to cause progression to OA and also inhibit cartilage regeneration after cartilage repair surgeries. During trans-signaling on cells that do not express the classical IL-6 receptor (as IL-6 receptor is present only on hepatocytes, some epithelial cells and some leukocytes), IL-6 complexes with its soluble receptor sIL-6Rα and this interaction induces a transition from acute to chronic inflammation by monocyte/macrophage recruitment instead of neutrophils, leading to apoptosis of neutrophils and monocyte propagation. In synovial fluid of patients with RA, OSM was highly expressed contributing to joint inflammation; in RA synovial explants, OSM induced cartilage degradation by increased production of MMP1. It was also shown that inhibition of OSM ameliorates joint inflammation in collagen-induced arthritis. During pathological inflammation, OSM was also shown to be released by neutrophils affecting endothelial cell function inducing adhesion of P-selectin in clathrin-coated pits.

However, multiple studies have demonstrated that this signaling cascade plays a broad role outside of inflammation. gp130 and its related cytokines are involved in the regulation of vital biological processes such as hematopoiesis, stemness, proliferation, differentiation, metabolism and regeneration. Different IL-6 family of cytokines induce dissimilar functional outcomes in cartilage. Thus, molecular signaling downstream of gp130 receptor and resulting outcomes are very context specific.

Engagement of gp130 leads to activation of various downstream pathways such as the JAK/STAT, SHP2/MAPK, and SRC (non-receptor tyrosine kinase). Much of the work focused on gp130-mediated signaling revolves around JAK/STAT and MAPK pathways, whereas little is known about the biological consequences of SRC downstream of gp130. Formerly, JAK/STAT3 signaling was identified as a vital pathway in degeneration and regeneration, suggesting that it drives both. However, STAT3 signaling within this network in the joint, providing insight that overexpression of STAT3 does not result in any ECM destruction and that it is actually highly expressed in developing anabolic fetal joints; instead, STAT3 activation by gp130 Y905 phosphorylation induced regeneration, cell proliferation and renewal. IL-6 cytokines can also drive activation of pro-inflammatory transcription factor NF-κB but the exact mechanism of NF-κB downstream of gp130 remains unclear. This transcription factor induces perpetuation of chronic inflammation/inflammaging controlling the expression of IL-lj and TNF-α, and is a major catabolic pathway in OA joints inducing cartilage destruction, synovial membrane inflammation and increased subchondral bone resorption.

Previously studied pathways induced by IL-6 family cytokines in various tissues cannot fully explain the magnitude of observed degenerative changes. This suggests that the divergence in outcomes of STAT3 and MAPK downstream of gp130 are cell-type and context-specific and are differentially regulated, which imparts the need for identification of a novel specific molecular mechanism within gp130 network to explain this dichotomy mediated by different IL-6 family of cytokines.

It has been thought that inflammation and recruitment of immune cells promotes regeneration of damaged tissue involving gp130. In mucosal injury, it has been shown that SRC is a downstream target of gp130 and association of SRC kinases (SFKs) with gp130 are essential to promote healing of the damaged epithelium. In vivo, SRC is essential to drive intestinal stem cell proliferation during tissue self-renewal and regeneration. The regenerative potential is mostly suggested in context of tumorigenesis and the role of SFKs is conditional to the specific tissue as it is also detrimental; dysregulated SRC-dependent signaling may lead to aberrant activation of infiltrating immune cells and cancer-precursor lesions. Inhibition of SFKs promote chondrogenic gene expression and phenotype, maintaining cartilage integrity, which is required for articular cartilage maintenance and for prevention of cartilage degradation. In spinal and bulbar muscular atrophy, it has been shown that SRC inhibition mitigated neuromuscular degeneration. Many SFKs are expressed in human RA synovium; they are highly predominant in lymphocytes, monocytes/macrophages and mast cells and are responsible for production of inflammatory cytokines, activation/migration of B cells, monocytes/macrophages, mast cells and inducing osteoclastogenesis. Furthermore, SRC kinase was highly activated in a rat model of collagen-induced arthritis and is implicated in bone resorption.

SFKs directly mediate activation of NF-κB. In acute inflammatory lung injury, SFKs play a critical role in LPS-induced NF-κB activation. In mice expressing a mutated kinase domain-truncated SRC, which develop severe osteopetrosis, inhibition of osteoclastogenesis was achieved through inhibition of NF-κB signaling. Studies have also demonstrated that interaction between SRC and IκB-α contributes to NF-κB activation in glomerular mesangial cells leading to renal inflammation. In cancer cells, NF-κB was shown to physically interact with SRC, and it has also been shown that in osteoclast progenitors, the regulatory subunit of the IKK complex, NEMO, interacts with SRC.

gp130 mediates downstream signaling via its intracellular residues that impact biological processes such as cell cycle regulation, proliferation and transcription. It is recognized that different residues are responsible for activation of various downstream pathways, including STAT3 and SHP2. It has been shown that STAT3 is predominantly activated by gp130 phosphorylated tyrosine residue Y905 and the second signaling pathway is mediated by phosphorylation of Y759 resulting in the recruitment of SHP2 triggering activation of MAPKs.

Previous studies have identified several downstream signaling pathways known to require gp130 residues as docking sites. Some pathways induced by gp130 activation are well-studied, including STAT3 and MAPK signaling, but current and previous studies has indicated that additional signaling modules downstream of gp130 may account for chronic inflammation and tissue degeneration in OA. However, the role of specific gp130 residues responsible for inducing these signaling cascades was not known. The disclosure identifies Y814 residue within gp130 as an initiating factor in upregulation of inflammatory SRC along with NF-κB, nominating this tyrosine as a therapeutic target for OA.

gp130-STAT3 signaling is highly upregulated in proliferative, anabolic fetal chondrocytes, and that LIF is highly expressed in developing human joints. Experiments demonstrated that Lifr-gp130-Stat3 signaling is required for homeostatic maintenance of chondroprogenitors in mice, and that genetic postnatal ablation of any members of this triad results in premature growth plate fusion and progressive changes in articular cartilage. Strikingly, genetic overexpression of STAT3 in postnatal chondrocytes did not induce an OA phenotype; moreover, chondrocyte hyperproliferation was observed in both growth plate and articular cartilage. Together, these data challenged previous oversimplified views on gp130-STAT3 signaling in cartilage tissue suggesting that activation of this pathway in OA may initially represent a regenerative attempt, but prolonged and excessive activation of this mechanism is likely to be detrimental.

The disclosure demonstrates that genetic or pharmacological inhibition of gp130-Y814 module designed to minimally interfere with the endogenous STAT3 signaling significantly improves regenerative outcomes in multiple tissues.

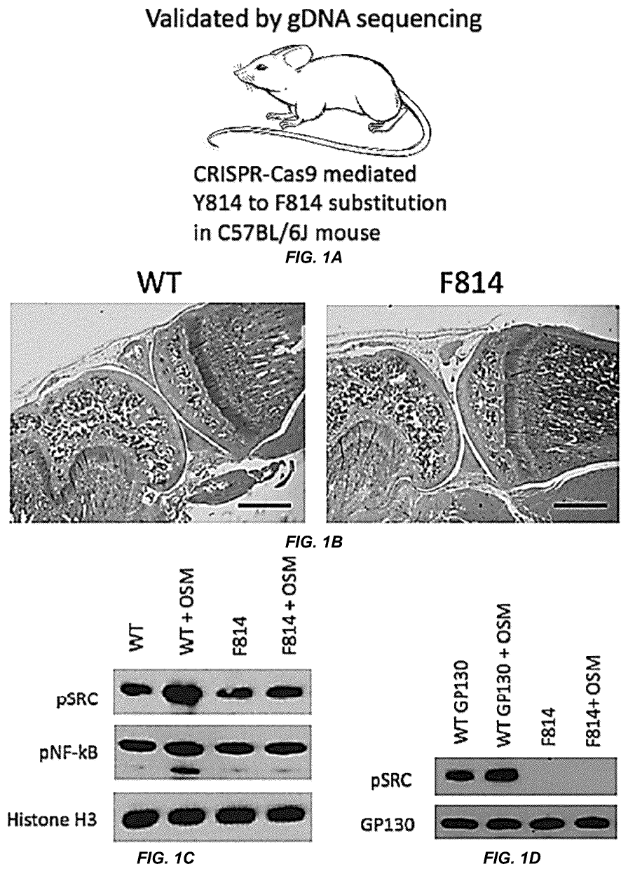

Through sequence substitutions in vitro, various amino acids were modified within the gp130 cytoplasmic domain. Tyrosine to phenylalanine modification of Y814 showed this residue activates SRC in pig and human chondrocytes. This residue was shown to decrease NF-κB expression within the same cell type, suggesting that manipulation of gp130 cannot only regulate SRC but also NF-κB in the context of the joint. In order to verify the function of this residue in vivo, a CRISPR/Cas9 homozygous murine model with a genetically modified Y814 was developed (FIG. 1A). The mutant mouse is viable, fertile, exhibits no striking morphological differences in the musculoskeletal tissues relative to wild type (WT) and has no organ pathology (FIG. 1B). Cells retrieved from mutant and WT spleens confirmed low SRC and NF-κB expression and these cells were unresponsive to stimulation by OSM (FIG. 1C). In addition, the mutant-derived cells revealed no endogenous interaction of SRC with gp130 as shown by co-immunoprecipitation (FIG. 1D). However, the mutation had no significant effect on STAT3 and MAPK signaling confirming that this residue is selective.

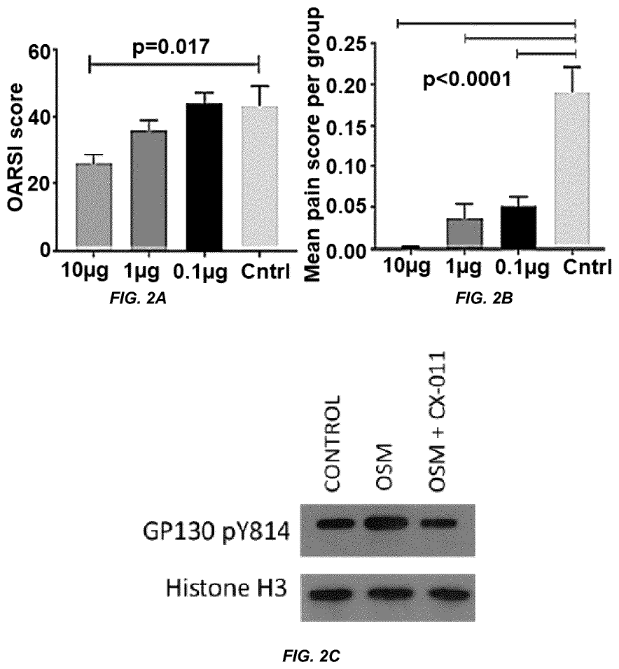

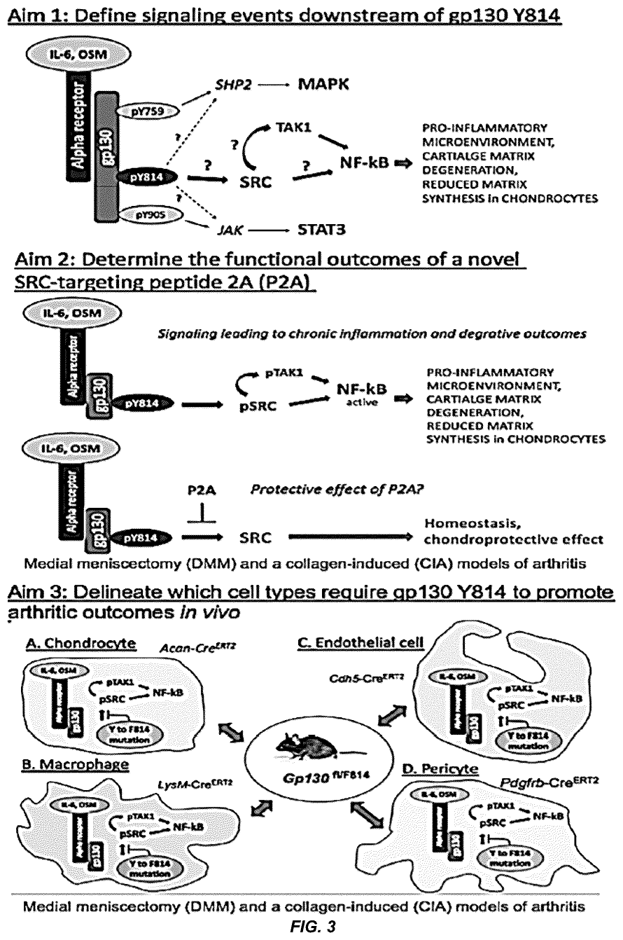

gp130 signaling can be modulated with a small molecule RCGD 423, which protects adult and pig articular cartilage from degeneration and promotes cartilage regeneration in rat medial meniscectomy and cartilage surface injury models. A more potent analog of this molecule termed CX-011; see International Pat. Publ. No. WO2019169135A1, was demonstrated to be biological active. A large-scale dog study demonstrated that CX-011 has a profound effect on alleviating pain and matrix catabolism in an OA dog joint (FIG. 2B,C). CX-011 is a potent inhibitor of OSM-stimulated activation of gp130 Y814 (FIG. 2A), indicating that this pathway is not only novel but plays a major role in OA.

gp130 signaling highlights the necessity of balance between pro- vs. anti-inflammatory and protective vs. pathogenic properties of gp130, which indicates that molecular crosstalk downstream of gp130 is cell-type specific and that different pathways and cytokines generate distinct functional outcomes. The data provided herein demonstrates a premise that gp130-SRC signaling axis plays a role in chronic inflammation, matrix catabolism and tissue degeneration which has significant clinical relevance.

The disclosure provides a gp130 signaling modality and a biological target that is responsible for mediating chronic inflammation and tissue degeneration. A gp130-dependent SRC-NF-κB signaling axis and the role of this mechanism has never been described in the framework of arthritis and skeletal tissue. The data shows that gp130 Y814 residue induces SRC-NF-κB signaling in the context of the joint. The disclosure shows that manipulation of this molecular pathway leads to inhibition of matrix catabolism and induced biosynthesis.

The disclosure also provides a Y814 CRISPR/Cas9 mutant homozygous mouse (F814) that can be used as an instrumental model for inflammatory and degenerative diseases potentially driven by inflammation and thus providing insight on the importance of this residue. This mutant mouse has showed peculiar responses to IL-6 cytokines with drastically reduced activation of SRC kinase in a gp130-dependent manner.

In addition, the disclosure provides a validated polyclonal pY814 antibody to be utilized in research for detection of SRC-NF-κB activity in tissues; the specificity of detection was examined in the mutant mouse which showed no Y814 activation and drastically diminished activation of both SRC and NF-κB by OSM when compared with WT cells. Further, various chemical inhibitors of gp130, SRC and NF-κB have previously been developed to decrease inflammatory outcomes; however, they are habitually nonspecific. Contrarily, synthetic peptides consisting of short sequence of amino acids are known to possess specificity and functional selectivity. In addition, since peptides mimic the endogenous motifs, they have low intrinsic immunogenicity and rapid clearance potential. These mimetic peptides are attractive candidates for inhibition of interactions involved in downstream signaling.

The disclosure provides SRC binding peptides and derivatives thereof. These peptides contain 4 amino acids resembling residues around Y814 of SEQ ID NO:26, which is essential for SRC-gp130 interaction; utilization of this peptide allows for modulation of protein function through controlled interference. This highly specific binding makes the peptides and derivatives thereof of the disclosure useful agent to modify disease associated protein interactions. Pilot studies in vitro have shown that these peptides are capable of inhibiting cytokine induced matrix catabolism and cartilage degradation confirming the importance of Y814. Based on the data, Y814 can serve as pivotal therapeutic target providing a way that pro-inflammatory signaling can be genetically and pharmacologically regulated in a highly selective manner. This modality has major molecular and clinical implications in inflammaging, autoimmune diseases and degenerative disorders.

The disclosure provides compositions and methods to modulate gp130 inflammatory activity and more particularly, inflammatory activity resulting from phosphorylation of Y814. The disclosure provides antibodies, peptides, small molecules and antibodies that interact with the gp130 Y814 module to inhibit the inflammatory cascade induced by activation of the Y814 module as well as diagnostics.

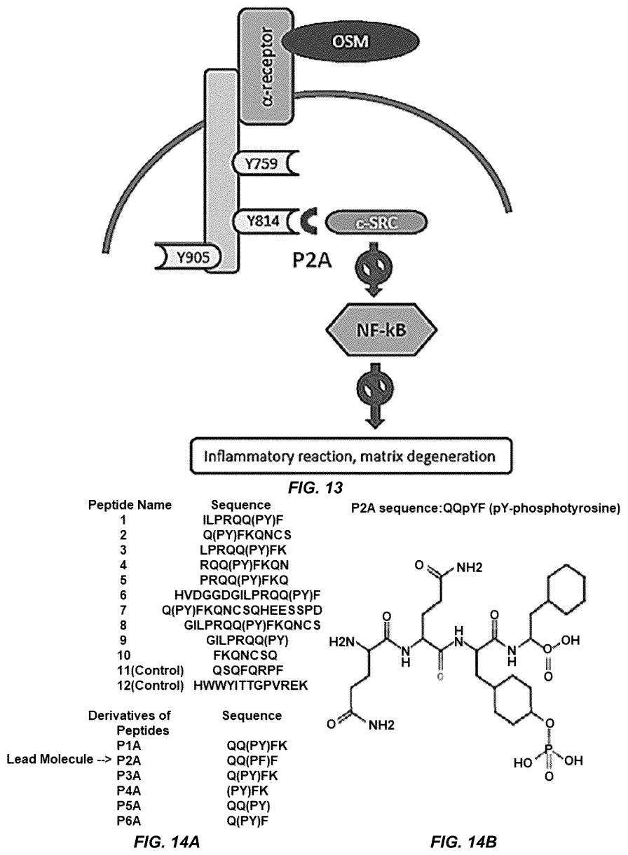

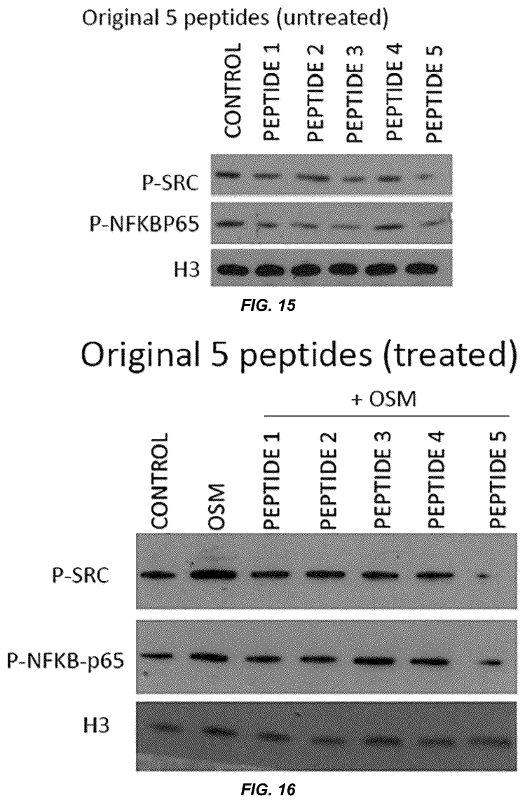

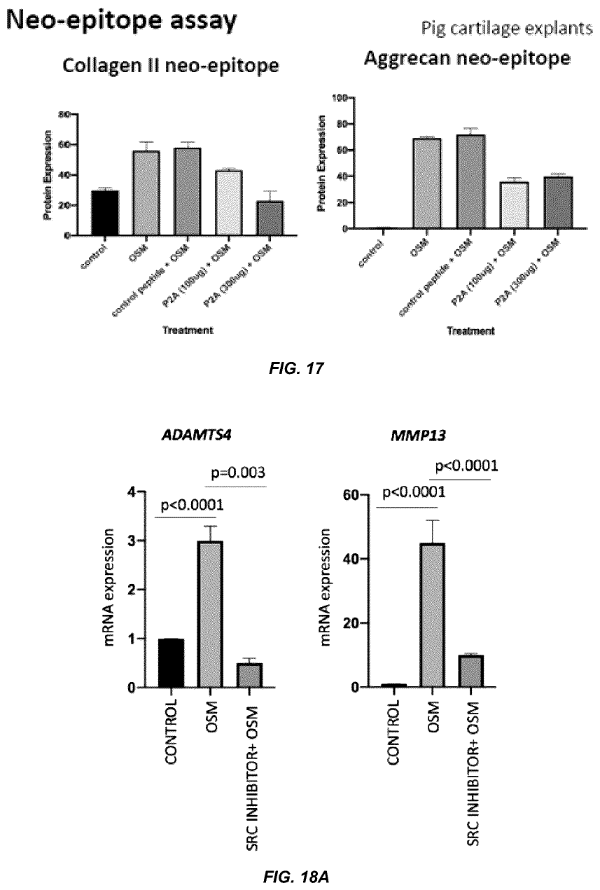

In one embodiment, the disclosure provides peptides and peptidomimetics that bind to c-SRC preventing activation of c-SRC by gp130 Y814. By inhibiting the interaction of gp130 Y814 with c-SRC the peptide and peptidomimetics of the disclosure inhibit OSM-induced pro-inflammatory cascade (see FIGS. 12-13).

The disclosure provides peptide and peptidomimetics that comprise a sequence QQ(pY)F motif as well as derivatives thereof. For example, Table 1 provides exemplary peptide/peptidomimetics and control peptides:

TABLE 1

| PEPTIDE NAME | SEQUENCE |
|---|---|
| 1 (SEQ ID NO: 61) | ILPROQ(pY)F |
| 2 (SEQ ID NO: 62) | Q(pY)FKQNCS |
| 3 (SEQ ID NO: 63) | LPRQQ(pY)FK |
| 4 (SEQ ID NO: 64) | RQQ(pY)FKQN |
| 5 (SEQ ID NO: 65) | PRQQ(pY)FKQ |
| 6 (SEQ ID NO: 66) | HVDGGDGILPROQ(pY)F |
| 7 (SEQ ID NO: 67) | Q(pY)FKQNCSQHEESSPD |
| 8 (SEQ ID NO: 68) | GILPRQQ(pY)FKQNCS |
| 9 (SEQ ID NO: 69) | GILPRQQ(pY) |
| 10 (SEQ ID NO: 70) | FKQNCSO |
| 11 (CONTROL) | OSOFORPE |
| 12 (CONTROL) (SEQ ID NO: 71) | HWWYITTGPVREK |
| Derivatives of peptide 5 | SEQUENCE |
| P1A (SEQ ID NO: 56) | QQ(pY)FK |
| P2A | QQ(PY)F |
| P3A | Q(pY)FK |
| P4A | (pY)FK |
| P5A | QQ(pY) |
| P6A | Q(pY)F |

The peptides/peptidomimetics can be 4-50 (e.g., 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33, 4-34, 4-35, 4-36, 4-37, 4-38, 4-39, 4-40, 4-41, 4-42, 4-43, 4-44, 4-45, 4-46, 4-47, 4-48, 4-49 or 4-50) amino acids and contain the sequence QQ(pY)F. In one embodiment, the peptide contains the sequence QQ(pY)F and further comprises 1 or more D-amino acids or 1 or more non-natural amino acids. In some embodiments, the peptide/peptidomimetic is circularized and contains a sequence QQ(pY)F that interacts with c-SRC. In still another embodiment, a peptide or peptidomimetic of the disclosure comprises the sequence QQ(pY)F directly or indirectly linked to a PTD domain. The QQ(pY)F can be separated from the PTD domain by a cleavable linker and may further comprise one or more D-amino acids. In other embodiments, QQ(pY)F can be conjugated or encapsulated in a cationic lipid or other cationically charged delivery vehicle to affect uptake of the peptide across cell membranes.

In one embodiment, a peptide of the disclosure comprises a sequence QQ(pY)F and optionally comprises 1-5, 1-10, 5-15 or 15-30 amino acids linked to either the N-terminal and/or C-terminal end of the QQ(pY)F peptide. The optional amino acids can comprise linkers and/or protein transductions domains (PTDs). In one embodiment, a linker may be linked to a protein transduction domain or a detectable label. In any of the foregoing, one or more of the amino acids linked to the N- and/or C-terminal ends can comprise L-, D- and/or unnatural amino acids. In any of the foregoing the peptide can bind to c-SRC and prevent the interaction of c-SRC with gp130.

As discussed elsewhere herein the peptide/peptidomimetics of the disclosure can be formulated for delivery to a site of, e.g., inflammation to inhibit the inflammatory cascade caused by binding of c-SRC to gp130 Y814.

In another embodiment, a molecule useful in the methods and compositions of the disclosure has the general formula I:

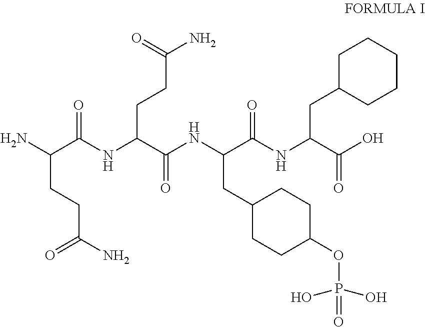

wherein additional amino acids can be present on either end of the structure of Formula I via peptide bonds.

As used herein, the term “amino acid” includes both natural and synthetic amino acids, and both D and L amino acids. “Natural amino acid” means any of the twenty primary, naturally occurring amino acids which typically form peptides, polypeptides, and proteins. “Synthetic amino acid” means any other amino acid, regardless of whether it is prepared synthetically or derived from a natural source. As used herein, “synthetic amino acid” also encompasses chemically modified amino acids, including but not limited to salts, derivatives (such as amides), and substitutions. Amino acids contained within the peptides of the disclosure, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention, as long as anti-HIV activity is maintained. The term also includes amino acids bearing a conventional amino protecting group (e.g. acetyl or benzyloxycarbonyl), as well as natural and unnatural amino acids protected at the carboxy terminus alkyl, phenyl or benzyl ester or amide; or as an alpha-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc, and documents cited therein). The peptide compositions of the disclosure may also include modified amino acids.

Unnatural amino acids useful for peptides/peptidomimetics include, but are not limited to, homolysine, homoarginine, homoserine, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, 2,4-diaminoisobutyric acid, desmosine, 2,2′-diaminopimeiic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylpentylglycine, naphthylalanine, ornithine, pentylglycine, thioproline, norvaline, tert-butylglycine, phenylglycine, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobuteric acid, (S)-2-amino-3-(1H-tetrazol-5-yl)propanoic acid, cyclopentylglycine, cyclohexylglycine, cyclopropylglycine, .eta.-.omega.-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, isopropyl-phenylglycine, (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, and (S)-2-amino-3-(1H-indazol-3-yl)propanoic acid and the D and L stereoisomers thereof.

Modified amino acid residues useful in the methods and compositions of the disclosure include, but are not limited to, those which are chemically blocked, reversibly or irreversibly, or chemically modified on their N-terminal amino group or their side chain groups, as for example, N-methylated D and L natural or unnatural amino acids or residues wherein the side chain functional groups are chemically modified to another functional group. For example, modified amino acids include without limitation, methionine sulfoxide; methionine sulfone; aspartic acid-(beta-methyl ester), a modified amino acid of aspartic acid; N-ethylglycine, a modified amino acid of glycine; or alanine carboxamide, and a modified amino acid of alanine. Unnatural amino acids may be purchased from Sigma Aldrich or other supplier.

“Analogs” is meant to include polypeptide variants which differ by one or more amino acid alterations, e.g., substitutions, additions or deletions of amino acid residues that still maintain one or more of the properties of the parent or starting polypeptide.

As used herein, the term “bridging moiety” refers to one or more components of a bridge formed between two adjacent or non-adjacent amino acids in a polypeptide. The bridging moiety may be of any size or composition. In some embodiments, a bridging moiety comprises one or more chemical bonds between two adjacent or non-adjacent amino acids. In some embodiments, such chemical bonds may be between one or more functional groups on adjacent or non-adjacent amino acids. In some embodiments, the bridging moiety comprises one or more features including, but not limited to, disulfide bonds, thioether bonds and cyclic rings. In some embodiments, the bridging moiety comprises a disulfide bond formed between two cysteine residues. In some embodiments, the bridging moiety comprises one or more thioether bonds. In some embodiments, bridging moieties comprise non-protein or non-peptide-based moieties, including, but not limited to, cyclic rings (including, but not limited to aromatic ring structures (e.g. xylyls)). Such bridging moieties may be introduced by reaction with reagents containing multiple reactive halides, including, but not limited to, poly(bromomethyl)benzenes, poly(bromomethyl)pyridines, poly(bromomethyl)alkylbenzenes and/or (E)-1,4-dibromobut-2-ene.

As used herein, a “conjugate” refers to any molecule or moiety appended to another molecule. In the disclosure, conjugates may be protein (amino acid) based or not. Conjugates may comprise lipids, small molecules, RNA, DNA, proteins, polymers, or combinations thereof. Functionally, conjugates may serve as targeting molecules or may serve as payload to be delivered to a cell, organ or tissue. Conjugates are typically covalent modifications introduced by reacting targeted amino acid residues or the termini of the polypeptide with an organic derivatizing agent that is capable of reacting with selected side-chains or terminal residues. Such modifications are within the ordinary skill in the art and are performed without undue experimentation.

Covalent modifications specifically include molecules in which proteins, peptides or polypeptides of the disclosure are bonded to a non-proteinaceous polymer. The non-proteinaceous polymer ordinarily is a hydrophilic synthetic polymer, i.e. a polymer not otherwise found in nature. However, polymers that exist in nature and are produced by recombinant or in vitro methods are useful, as are polymers which are isolated from nature. Hydrophilic polyvinyl polymers fall within the scope of this disclosure, e.g. polyvinylalcohol and polyvinylpyrrolidone. The proteins, peptides or polypeptides may be linked to various non-proteinaceous polymers, such as polyethylene glycol (PEG), polypropylene glycol or polyoxyalkylenes, in the manner set forth in U.S. Pat. Nos. 4,640,835; 4,496,689; 4,301,144; 4,670,417; 4,791,192 or 4,179,337.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

As used herein, the term “cyclic peptide mimetic” or “cyclic polypeptide mimetic” refers to a peptide mimetic that has as part of its structure one or more cyclic features such as a loop, bridging moiety, and/or an internal linkage.

“Disease” or “condition” refer to a state of being or health status of a patient or subject capable of being treated with the compounds/molecules or methods provided herein. Disease as used herein may refer to inflammatory diseases and disorders and immune diseases and disorders such as cartilage degenerative disease, joint surface injury or arthritis (including rheumatoid arthritis), psoriasis, inflammatory bowel disease, aging, lupus, rosacea and the like.

The term “gp130” as used herein refers to glycoprotein 130, a cell surface receptor that is expressed ubiquitously in the body. Activation of gp130 is essential for several physiological functions, including but not limited to, acute-phase response to injury and infection, fertility, metabolism, hematopoiesis, neuroprotection, anti-angiogenesis, and melanoma and tumor cell suppression. Gp130 is activated by a ligand from the IL-6 family of cytokines, including but not limited to, IL-6, IL-11, leukemia inhibitory factor (LIF), Oncostatin M (OSM), ciliary neurotrophic factor (CNTF), cardiotrophin-1 (CT-1) and cardiotrophin-like cytokine (CLC). Activation of gp130 signaling may be direct, i.e. activation may be triggered by binding of the ligand directly to gp130 (i.e., IL-6 or IL-11, which result in gp130-homodimerization). Activation of gp130 signaling may also be indirect by binding of the ligand to another cell surface receptor, which forms a complex with gp130, thereby activating it. LIF, CT-1, CNTF, OSM and CLC form heterodimers of gp130 and LIFR, whereas OSM may also form a heterodimer of gp130 and OSMR. Therefore, LIF, CT-1, CNTF, OSM and CLC may activate gp130 signaling directly, by binding gp130 first, or indirectly, by binding LIFR/OSMR and then recruiting gp130 to the complex. The ligands of the IL-6 cytokine family trigger the JAK/STAT pathway, the first event of which is the ligand-induced homo- or hetero-dimerization of signal-transducing receptor subunits. All IL-6-type cytokines recruit gp130 to their receptor complexes. They either signal via gp130 alone or in combination with LIFR or OSMR, which are all able to activate Jaks and to recruit STAT proteins. The terms “gp130 receptor,” “gp130,” gp130 protein,” “IL6ST receptor,” “IL6ST” or “IL6ST protein” are herein used interchangeably and according to their common, ordinary meaning (e.g., transmembrane protein “glycoprotein 130”) and refer to proteins of the same or similar names and functional fragments and homologs thereof. The term includes any recombinant or naturally occurring form of, or variants thereof that maintain gp130 activity (e.g. within at least 30%, 40%, 50%, 60%, 70%, 80%, 90%, 95%, or 100% activity compared to gp130). In embodiments, the gp130 receptor has at least 80%, 85%, 90%, 95%, 96%, 97%, 98%, 99% or 100% sequence identity to SEQ ID NO:26 or a functional fragment thereof (e.g. 700 contiguous amino acids of SEQ ID NO:26, 750 contiguous amino acids of SEQ ID NO:26, 800 contiguous amino acids of SEQ ID NO:26, 850 contiguous amino acids of SEQ ID NO:26, 870 contiguous amino acids of SEQ ID NO:26, 880 contiguous amino acids of SEQ ID NO:26, 890 contiguous amino acids of SEQ ID NO: 26, 900 contiguous amino acids of SEQ ID NO:26 or 910 contiguous amino acids of SEQ ID NO:26).

SEQ ID NO: 26 (homo sapiens):
MLTLQTWLVQ ALFIFLTTES TGELLDPCGY ISPESPVVQL HSNFTAVCVL KEKCMDYFH
VNANYIVWKT NHFTIPKEQY TIINRTASSV TFTDIASLNI QLTCNILTFG QLEQNVYGI
TIISGLPPEK PKNLSCIVNE GKKMRCEWDG GRETHLETNF TLKSEWATHK FADCKAKRD
TPTSCTVDYS TVYFVNIEVW VEAENALGKV TSDHINFDPV YKVKPNPPHN LSVINSEEL
SSILKLTWTN PSIKSVIILK YNIQYRTKDA STWSQIPPED TASTRSSFTV QDLKPFTEY
VERIRCMKED GKGYWSDWSE EASGITYEDR PSKAPSFWYK IDPSHTQGYR TVQLVWKTL
PFEANGKILD YEVTLTRWKS HLQNYTVNAT KLTVNLTNDR YLATLTVRNL VGKSDAAVLT
IPACDFQATH PVMDLKAFPK DNMLWVEWTT PRESVKKYIL EWCVLSDKAP CITDWQQEDG
TVHRTYLRGN LAESKCYLIT VTPVYADGPG SPESIKAYLK QAPPSKGPTV RTKKVGKNEA
VLEWDQLPVD VQNGFIRNYT IFYRTIIGNE TAVNVDSSHT EYTLSSLTSD TLYMVRMAAY
TDEGGKDGPE FTFTTPKFAQ GEIEAIVVPV CLAFLLTTLL GVLFCENKRD LIKKHIWPNV
PDPSKSHIAQ WSPHTPPRHN FNSKDQMYSD GNFTDVSVVE IEANDKKPFP EDLKSLDLFK
KEKINTEGHS SGIGGSSCMS SSRPSISSSD ENESSQNTSS TVQYSTVVHS GYRHQVPSVQ
VFSRSESTQP LLDSEERPED LQLVDHVDGG DGILPRQQYF KQNCSQHESS PDISHFERSK
QVSSVNEEDF VRLKQQISDH ISQSCGSGQM KMFQEVSAAD AFGPGTEGQV ERFETVGMEA
ATDEGMPKSY LPQTVRQGGY MPQ
SEQ ID NO: 27 (Mus musculus):
MSAPRIWLAQ ALLFFLTTES IGQLLEPCGY IYPEFPVVQR GSNFTAICVL KEACLQHYYV
NASYIVWKTN HAAVPREQVT VINRTTSSVT FTDVVLPSVQ LTCNILSFGQ IEQNVYGVTM
LSGFPPDKPT NLTCIVNEGK NMLCQWDPGR ETYLETNYTL KSEWATEKFP DCQSKHGTSC
MVSYMPTYYV NIEVWVEAEN ALGKVSSESI NFDPVDKVKP TPPYNLSVTN SEELSSILKL
SWVSSGLGGL LDLKSDIQYR TKDASTWIQV PLEDTMSPRT SFTVQDLKPF TEYVFRIRSI
KDSGKGYWSD WSEEASGTTY EDRPSRPPSF WYKTNPSHGQ EYRSVRLIWK ALPLSEANGK
ILDYEVILTQ SKSVSQTYTV TGTELTVNLT NDRYVASLAA RNKVGKSAAA VLTIPSPHVT
AAYSVVNLKA FPKDNLLWVE WTPPPKPVSK YILEWCVLSE NAPCVEDWQQ EDATVNRTHL
RGRLLESKCY QITVTPVFAT GPGGSESLKA YLKQAAPARG PTVRTKKVGK NEAVLAWDQI
PVDDQNGFIR NYSISYRTSV GKEMVVHVDS SHTEYTLSSL SSDTLYMVRM AAYTDEGGKD
GPEFTFTTPK FAQGEIEAIV VPVCLAFLLT TLLGVLFCEN KRDLIKKHIW PNVPDPSKSH
IAQWSPHTPP RHNFNSKDQM YSDGNFTDVS VVEIEANNKK PCPDDLKSVD LFKKEKVSTE
GHSSGIGGSS CMSSSRPSIS SNEENESAQS TASTVQYSTV VHSGYRHQVP SVQVFSRSES
TQPLLDSEER PEDLQLVDSV DGGDEILPRQ PYFKQNCSQP EACPEISHFE RSNQVLSGNE
EDFVRLKQQQ VSDHISQPYG SEQRRLFQEG STADALGTGA DGQMERFESV GMETTIDEEI
PKSYLPQTVR QGGYMPQ

The term “tyrosine residue corresponding to the tyrosine residue at position 814 of human gp130 protein” as used herein means a tyrosine residue in gp130 protein or homolog of a transgenic animal of the disclosure (e.g., a mouse), wherein said tyrosine residue corresponds to the tyrosine residue located at position 814 in human gp130 protein. The term “region comprising a tyrosine residue corresponding to the tyrosine residue at position 814 of human gp130 protein” means a region of the amino acid residues in the gp130 protein of the transgenic animal of the disclosure, wherein said region comprises said tyrosine residue corresponding to the tyrosine residue at position 814 of human gp130 protein. In a gp130 variant expressed in the transgenic animal of the disclosure, a tyrosine residue, which corresponds to the tyrosine residue at position 814 of human gp130 protein, is substituted or deleted, and/or one or more amino acid residue are substituted, inserted or deleted in a region comprising said corresponding tyrosine residue. In a specific embodiment, a residue corresponding to the tyrosine residue at position 814 of human gp130 refers to Y812 of SEQ ID NO:27.

A gp130 variant expressed in the transgenic animal which is used in the method of screening of the disclosure. A transgenic animal of the disclosure comprising a substitution of an amino acid phenylalanine for tyrosine at a residue corresponding to Y814 of SEQ ID NO:26 provides a phenotype showing reduced inflammatory cascade induced by OSM compared to a wild-type animals.

“Homology” or “identity” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

“Inflammation” refers to a complex biological response of a body to a stimulus (e.g., a pathogen, cellular damage or an irritant). Inflammation, when prolonged, can lead to an inflammatory disease or disorder. Factors elicited during an inflammatory reaction include pro-inflammatory cytokines (e.g., TNF-α, IL-1, INF-γ, MCP-1), cellular migration (e.g., monocytes, macrophages, lymphocytes, plasma cells) and serum proteins (e.g., serum amyloid A (SAA) and serum amyloid P (SAP)). Inflammation can be local (e.g., vascular inflammation) or systemic.

“Inflammatory disorder” or “inflammatory disease” refers to a condition characterized by inflammation in a cell, tissue or body. Inflammatory diseases and disorders include, but are not limited to, hypersensitivities (e.g., allergies), asthma, autoimmune disease (e.g., rheumatoid and osteo arthritis, lupus, multiple sclerosis), cancer, diabetes, inflammatory bowel disease (IBD) or cardiovascular disease (e.g., atherosclerosis), NAFLD, NASH, hepatitis, fibrosis, and cirrhosis.

As used herein, a “mimetic” refers to a molecule which exhibits some of the properties or features of another molecule. A “peptide mimetic” (also referred to as a peptidomimetic) is a mimetic in which the molecule contains non-peptidic structural elements that are capable of mimicking or antagonizing the biological action(s) of a natural peptide. A peptidomimetic may have many similarities to natural peptides, such as: amino acid side chains that are not found among the known 20 proteinogenic amino acids, non-peptide-based linkers used to effect cyclization between the ends or internal portions of the molecule, substitutions of the amide bond hydrogen moiety by methyl groups (N-methylation) or other alkyl groups, replacement of a peptide bond with a chemical group or bond that is resistant to chemical or enzymatic treatments, N- and C-terminal modifications, and conjugation with a non-peptidic extension (such as polyethylene glycol, lipids, carbohydrates, nucleosides, nucleotides, nucleoside bases, various small molecules, or phosphate or sulfate groups).

A “peptide” is a compound comprised of amino acid residues covalently linked by peptide bonds.

The term “pharmaceutically acceptable” as in pharmaceutically acceptable salt or pharmaceutically acceptable counter ion, refers to compounds, salts, or ions that are tolerated by a subject for topical, or internal use.

The term “pharmaceutically acceptable salt” refers to making a salt formation of a compound disclosed herein. Salt formation can be used as a means of varying the properties of the compounds disclosed herein, for example, to increase or decrease solubility of the compounds, to improve stability of the compounds, to reduce toxicity of the compounds, and/or to reduce the hygroscopicity of the compounds. There are a wide range of chemically diverse acids and bases, with a range of pKa values, molecular weights, solubilities and other properties, that can used for making pharmaceutically acceptable salts of the compounds disclosed herein. Examples of pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the disclosure can form pharmaceutically acceptable salts with various amino acids. Examples of pharmaceutically acceptable base addition salts include, but are not limited to, aluminum, calcium, lithium, magnesium, potassium, sodium, zinc, and diethanolamine salts. For additional examples of pharmaceutical salts that can used to practice this disclosure, see P. H. Stahl and C. G. Wermuth (eds.), Pharmaceutical Salts: Properties, Selection, and Use (2d ed. 2011) Wiley and Sons Publisher, ISBN: 978-3-90639-051-2.

The term “pharmaceutically acceptable counter ion” either refers to pharmaceutically acceptable cations including, but not limited to, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyls, i.e., including, methyl, ethyl, or hydroxyethyl, specifically, trimethyl ammonium, triethyl ammonium, and triethanol ammonium cations); or pharmaceutically-acceptable anions including, but not limited to, halides (e.g., Cl⁻, Br⁻), sulfate, acetates (e.g., acetate, trifluoroacetate), ascorbates, aspartates, benzoates, citrates, and lactate.

As used herein when referring to polypeptides the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the disclosure.

A “subject” generally refers to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

The term “substantially” as used to modify a term means that the modified term includes minor variations in size, purity, structure and the like by only a minor amount. Accordingly, “substantially homogenous in size” means that the material does not vary by more than 1%, 5%, 10%, 20% or 30% (or any value there between) in size from an average size.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the terms “termini or terminus” when referring to polypeptides refers to an extremity of a peptide or polypeptide. Such extremity is not limited only to the first or final site of the peptide or polypeptide but may include additional amino acids in the terminal regions. The polypeptide based molecules of the disclosure may be characterized as having both an N-terminus (terminated by an amino acid with a free amino group (NH₂)) and a C-terminus (terminated by an amino acid with a free carboxyl group (COOH)).

A “therapeutically effective amount,” refers to an amount of a compound, molecule or composition of the disclosure that reduces a symptom or symptoms (and grammatical equivalents of this phrase) or the severity of or frequency of the symptom(s), or elimination of the symptom(s) associated with a disease or disorder to be treated. A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

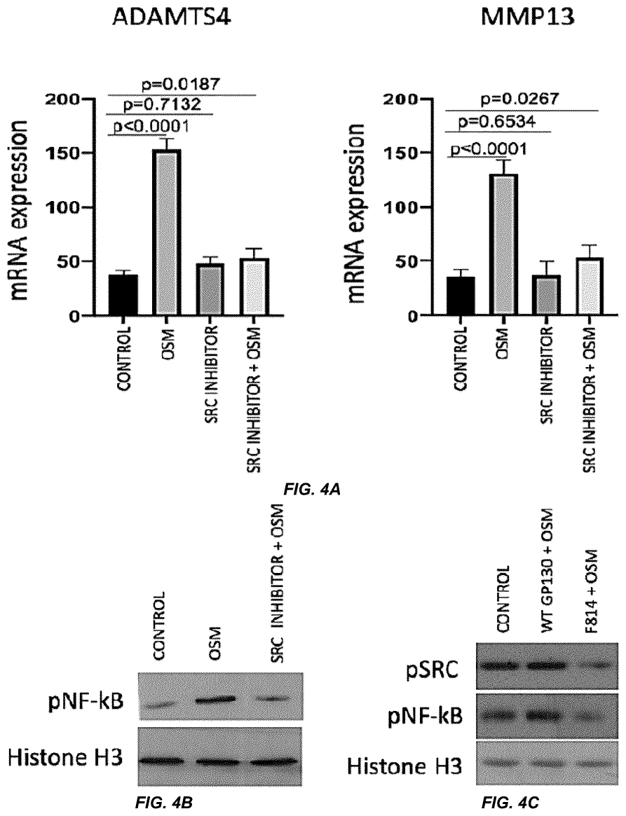

Initial studies show that chemical inhibition of SRC kinase had the most dramatic effect on preventing cartilage degeneration induced by the IL-6 family cytokine, OSM, by inhibiting transcription of matrix degrading enzymes ADAMTS4 and MMP13 (FIG. 4A). Persistent SRC and NF-κB upregulation are known to play a role in OA and RA pathogenesis, and experiments show that these proteins are highly expressed in the OA joint. SRC is also known to activate NF-κB signaling, and the data show that in the joint, SRC inhibition was able to downregulate NF-κB activation (FIG. 4B). Aberrant NF-κB activation is detrimental in cartilage as it drives a pro-inflammatory, catabolic responses that result in increased production of proteinases and IL-6 cytokines.

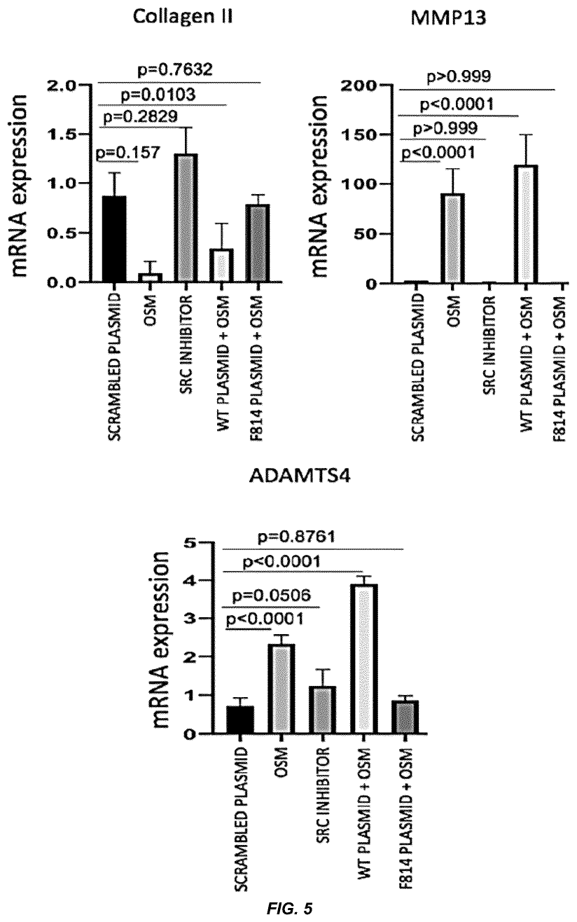

The data demonstrate that functional modification of tyrosine (Y) to phenylalanine (F) in gp130 Y814 residue via mutant plasmid (F814) is responsible for SRC and NF-κB downregulation in ATDC5 transfected cells relative to the control WT gp130 plasmid (FIG. 4C); in the same cells, decreased expression of matrix degenerating genes and increased biosynthesis was observed (FIG. 5). To verify this effect in vivo, a Y814 mutant (F814) mouse (point mutation: Y→F) was generated. The transgenic mouse model confirmed that substitution of this residue resulted in downregulation of SRC and NF-κB activation while other signaling pathways downstream of gp130 were minimally affected.

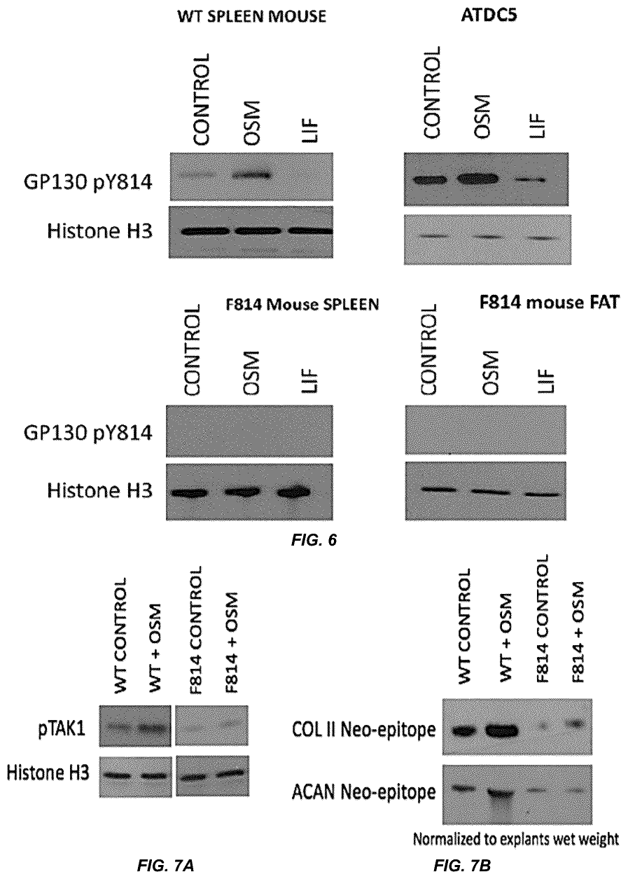

Furthermore a polyclonal antibody of the disclosure specific against pY814 confirmed marked activation of this residue in response to OSM treatment in various cell types in WT, but not in the F814, mouse; however, LIF had no effect on activation of this residue in WT suggesting that different IL-6 cytokines might not require this residue for downstream signaling (FIG. 6). Based on these data it was hypothesized that the gp130 residue Y814 is responsible for modulating a SRC-signaling cascade in different cell types and promotes matrix catabolism and degeneration, and that manipulation of this residue prevents matrix degradation and promote regeneration of damaged tissue.

A test compound which may be used in the screening method of the disclosure includes, for example, peptides, proteins, non-peptide compounds, antisense DNAs, antisense RNAs, synthetic compounds, fermented products, cell extracts, plant extracts, mammal tissue extracts, plasma, serum and the like, and these test compounds may be novel or known.

The means for administration of a test compound to a non-human transgenic organism in the screening method of the disclosure includes, for example, oral administration, intravenous injection, intradermal injection, intramuscular injection and the like. Those skilled in the art can appropriately select the dosage of a test compound according to the route of administration, the property of the test compound and the like. The administration of a test compound may be started prior to the timing of the onset of immune abnormality or the disease for screening of prophylactic drugs, whereas after the disease is macroscopically presented for screening of therapeutic agents.

In the screening method of the disclosure, male and female wild and the non-human transgenic organisms of the disclosure are divided into groups, and the animals, with or without administration of a test compound, are visually observed and scored for the severity of the symptom. Blood is collected from the organisms and subjected to blood cell counting and serological a test, additionally a urine test and the like are also conducted. After a certain period of observation, X-ray photogram analysis, pathological analysis and immunological analysis are conducted.

Tissues or cells can be isolated from the non-human transgenic organisms of the disclosure and subjected to analyses in vitro to assess preventing or therapeutic effect of a test compound, changes in the cellular phenotype resulting from the genetic modification for changes in cell growth and cytokine production and the like.

By virtue of their size and complexity, peptides are able to form numerous, highly specific contacts with their biological targets/cognates and can show a high level of selectivity for the correct or desired target as compared to a closely related target within the same family. Numerous peptides have been developed into effective drugs. These include, but are not limited to, insulin, glucagon-like peptide 1 (GLP-1), somatostatin, vasopressin, cyclosporine A, and the like. In a case such as insulin, the therapeutic peptide can be identical to the naturally occurring molecule (i.e. that which circulates in humans and is considered “wild-type” in the human population). In many other cases, the peptide is not suitable or sub-optimal for therapeutic use due to a short circulating half-life that is often due to metabolic instability in the body. In these cases a modified or a variant form of the peptide (peptidomimetic) is used which results in improved pharmacokinetic and pharmacodynamic behavior.

Peptides are typically limited to non-oral routes of administration. In nearly all cases, peptides and peptidomimetics must be delivered by injection, since even very short peptides (e.g., peptides with 4-10 amino acid residues) are incapable or poorly capable of passing through the cell membranes lining the intestinal tract. For efficient oral availability, drugs typically need to pass through both the luminal and basolateral membranes of gut epithelial cells in order to enter the systemic circulation. The poor membrane permeability and lack of oral bioavailability of peptides significantly limits their therapeutic use. In some instances, the permeability can be modified by linking the peptide to cell penetrating peptides (CPPs; sometimes referred to as protein transduction domains (PTDs)). PTDs are known in the art and include a number of peptide sequences.

Cellular delivery can be accomplished by macromolecule fusion of “cargo” biological agents (in this case the peptides of the disclosure) to a cationic Peptide Transduction Domain such as TAT or (Arg₈) (Snyder and Dowdy, 2005, Expert Opin. Drug Deliv. 2, 43-51). PTDs can be used to deliver a wide variety of macromolecular cargo, including the peptides described herein. Cationic PTDs enter cells by macropinocytosis, a specialized form of fluid phase uptake that all cells perform.

Biophysical studies on model vesicles suggest that cargo escape from macropinosome vesicles into the cytoplasm, thus requiring a pH decrease (Magzoub et al., 2005, Biochemistry 44, 14890-14897). The cationic charge of the PTDs is essential for the molecules to traverse the cell membrane.

The discovery of several proteins which could efficiently pass through the plasma membrane of eukaryotic cells has led to the identification of a class of proteins from which peptide transduction domains have been derived. The best characterized of these proteins are the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), and more recently the cationic N-terminal domain of prion proteins. Exemplary PTD sequences are provided in Table A. The disclosure further provides for one or more of the PTDs listed in Table A or other PTDs known in the art (see, e.g., Joliot et al., Nature Cell Biology, 6(3):189-196, 2004) to be conjugated to the peptides/peptidomimetics disclosed herein. Strategies for conjugation include the use of a bifunctional linker that includes a functional group that can be cleaved by the action of an intracellular enzyme.

TABLE A

| PDP | Sequence | SEQ ID NO. |
|---|---|---|
| TAT | RKKRRQRRR | SEQ ID NO.: 1 |
| Penetratin | RQIKIWFQNRRMKWKK | SEQ ID NO.: 2 |
| Buforin II | TRSSRAGLQFPVGRVHRLLRK | SEQ ID NO.: 3 |
| Transportan | GWTLNSAGYLLGKINKALAALAKKIL | SEQ ID NO.: 4 |
| MAP (model | KLALKLALKALKAALKLA | SEQ ID NO.: 5 |
| amphipathic | | |
| peptide) | | |
| K-FGF | AAVALLPAVLLALLAP | SEQ ID NO.: 6 |
| Ku70 | VPMLK-PMLKE | SEQ ID NO.: 7 |
| Prion | MANLGYWLLALFVTMWTDVGLCKKRPKP | SEQ ID NO.: 8 |
| pVEC | LLIILRRRIRKQAHAHSK | SEQ ID NO.: 9 |
| Pep-1 | KETWWETWWTEWSQPKKKRKV | SEQ ID NO.: 10 |
| SynB1 | RGGRLSYSRRRFSTSTGR | SEQ ID NO.: 11 |
| Pep-7 | SDLWEMMMVSLACQY | SEQ ID NO.: 12 |
| (phage display) | | |
| HN-1 | TSPLNIHNGQKL | SEQ ID NO.: 13 |
| (phage display) | | |

Exemplary auxiliary moieties, which comprise TAT peptides that can be conjugated to any of the nucleic acid constructs described herein are provided in Table B.

TABLE B
Sequence (N′ to C′)

PEG-(PTD)
GG-(PTD)-PEG-(PTD)
PEG-(PTD)-PEG-(PTD)
GG-(PTD)-PEG-PEG-PEG-(PTD)
PEG-(PTD)-PEG-PEG-PEG-(PTD)
GG-(PTD)-PEG-(PTD)-PEG-(PTD)
GG-(PTD)-PEG-PEG-PEG-(PTD)-PEG-PEG-PEG-(PTD)
PEG = a poly(ethyleneglycol) linker having six repeat units

Thus, PTDs that can be conjugated to a peptide of the disclosure include, but are not limited to, AntHD, TAT, VP22, cationic prion protein domains, and functional fragments thereof.

In a particular embodiment, the disclosure therefore provides methods and compositions that combine the use of PTDs, such as TAT and poly-Arg, with a peptide/peptidomimetic disclosed herein to facilitate the targeted uptake of the construct into and/or release within targeted cells. The peptides disclosed herein therefore provide methods whereby a therapeutic activity of the peptide can be targeted to be delivered in certain cells comprising one or more PTDs linked to the peptide.

In general, the delivery domain that is linked to a peptide disclosed herein can be nearly any synthetic or naturally-occurring amino acid sequence which assists in the intracellular delivery of a nucleic construct disclosed herein into targeted cells. For example, transfection can be achieved in accordance with the disclosure by use of a peptide transduction domain, such as an HIV TAT protein, or fragment thereof, that is covalently linked to a peptide/peptidomimetic of the disclosure. Alternatively, the peptide transduction domain can comprise the Antennapedia homeodomain or the HSV VP22 sequence, the N-terminal fragment of a prion protein or suitable transducing fragments thereof such as those known in the art.

The type and size of the PTD will be guided by several parameters including the extent of transfection desired. Typically the PTD will be capable of transfecting at least about 20%, 25%, 50%, 75%, 80% or 90%, 95%, 98% and up to, and including, about 100% of the cells. Transfection efficiency, typically expressed as the percentage of transfected cells, can be determined by several conventional methods.

PTDs will manifest cell entry and exit rates (sometimes referred to as k₁ and k₂, respectively) that favor at least picomolar amounts of a nucleic acid construct disclosed herein into a targeted cell. The entry and exit rates of the PTD and any cargo can be readily determined or at least approximated by standard kinetic analysis using detectably-labeled fusion molecules. Typically, the ratio of the entry rate to the exit rate will be in the range of between about 5 to about 100 up to about 1000.

In one embodiment, a PTD useful in the methods and compositions of the disclosure comprises a peptide or polypeptide featuring substantial alpha-helicity. It has been discovered that transfection is optimized when the PTD exhibits significant alpha-helicity. In another embodiment, the PTD comprises a sequence containing basic amino acid residues that are substantially aligned along at least one face of the peptide or polypeptide. A PTD domain useful in the disclosure may be a naturally occurring peptide or polypeptide or a synthetic peptide or polypeptide.

In another embodiment, the PTD comprises an amino acid sequence comprising a strong alpha helical structure with arginine (Arg) residues down the helical cylinder.

In yet another embodiment, the PTD domain comprises a peptide represented by the following general formula: B_{P1}—X_{P1}—X_{P2}—X_{P3}—B_{P2}—X_{P4}—X_{P5}—B_{P3} (SEQ ID NO:14) wherein B_{P1}, B_{P2}, and B_{P3} are each independently a basic amino acid, the same or different; and X_{P1}, X_{P2}, X_{P3}, X_{P4}, and X_{P5} are each independently an alpha-helix enhancing amino acid, the same or different.

In another embodiment, the PTD domain is represented by the following general formula: B_{P1}—X_{P1}—X_{P2}—B_{P2}—B_{P3}—X_{P3}—X_{P4}—B_{P4} (SEQ ID NO:15) wherein B_{P1}, B_{P2}, B_{P3}, and B_{P4} are each independently a basic amino acid, the same or different; and X_{P1}, X_{P2}, X_{P3}, and X_{P4} are each independently an alpha-helix enhancing amino acid the same or different.

Additionally, PTD domains comprise basic residues, e.g., lysine (Lys) or arginine (Arg), and further can include at least one proline (Pro) residue sufficient to introduce “kinks” into the domain. Examples of such domains include the transduction domains of prions. For example, such a peptide comprises KKRPKPG (SEQ ID NO:16).

In one embodiment, the domain is a peptide represented by the following sequence: X_{P}—X_{P}—R—X_{P}—(P/X_{P})—(B_{P}/X_{P})—B_{P}—(P/X_{P})—X_{P}—B_{P}—(B_{P}/X_{P}) (SEQ ID NO:17), wherein X is any alpha helical promoting residue such as alanine; P/X_{P} is either proline or X_{P} as previously defined; B_{P} is a basic amino acid residue, e.g., arginine (Arg) or lysine (Lys); R is arginine (Arg) and B_{P}/X_{P} is either B_{P} or X_{P} as defined above.

In another embodiment the PTD is cationic and consists of between 7 and 10 amino acids and contains the formula KX_{P1}RX_{P2}X_{P1} (SEQ ID NO:18), wherein X_{P1} is R or K and X_{P2} is any amino acid. An example of such a peptide comprises RKKRRQRRR (SEQ ID NO:1). In another example, the PTD is a cationic peptide sequence having 5-10 arginine (and/or lysine) residues over an length of 5-15 amino acids.

Additional delivery domains in accord with this disclosure include a TAT fragment that comprises at least amino acids 49 to 56 of TAT up to about the full-length TAT sequence. A TAT fragment may include one or more amino acid changes sufficient to increase the alpha-helicity of the fragment. In some instances, the amino acid changes introduced will involve adding a recognized alpha-helix enhancing amino acid. Alternatively, the amino acid changes will involve removing one or more amino acids from the TAT fragment that impede alpha helix formation or stability. In a more specific embodiment, the TAT fragment will include at least one amino acid substitution with an alpha-helix enhancing amino acid. Typically the TAT fragment will be made by standard peptide synthesis techniques although recombinant DNA approaches may be used in some cases. In one embodiment, the substitution is selected so that at least two basic amino acid residues in the TAT fragment are substantially aligned along at least one face of that TAT fragment. In a more specific embodiment, the substitution is chosen so that at least two basic amino acid residues in the TAT 49-56 sequence are substantially aligned along at least one face of that sequence.

Additional transduction proteins (PTDs) that can be used in the compositions and methods of the disclosure include the TAT fragment in which the TAT 49-56 sequence has been modified so that at least two basic amino acids in the sequence are substantially aligned along at least one face of the TAT fragment. Illustrative TAT fragments include at least one specified amino acid substitution in at least amino acids 49-56 of TAT which substitution aligns the basic amino acid residues of the 49-56 sequence along at least one face of the segment and typically the TAT 49-56 sequence.

Also included are chimeric PTD domains. Such chimeric PTDs include parts of at least two different transducing proteins. For example, chimeric PTDs can be formed by fusing two different TAT fragments, e.g., one from HIV-1 and the other from HIV-2 or one from a prion protein and one from HIV.

Peptide linkers that can be used in the constructs and methods of the disclosure will typically comprise up to about 20 or 30 amino acids, commonly up to about 10 or 15 amino acids, and still more often from about 1 to 5 amino acids. The linker sequence is generally flexible so as not to hold the fusion molecule in a single rigid conformation. The linker sequence can be used, e.g., to space the PTD domain from the peptide/peptidomimetic to be delivered. For example, the peptide linker sequence can be positioned between the peptide transduction domain and the therapeutic peptide/peptidomimetic domain, e.g., to provide molecular flexibility. The length of the linker moiety is chosen to optimize the biological activity of the peptide or polypeptide comprising, for example, a PTD domain fusion construct and can be determined empirically without undue experimentation. Examples of linker moieties are -Gly-Gly-, GGGGS (SEQ ID NO:19), (GGGGS)_{N} (SEQ ID NO:20), GKSSGSGSESKS (SEQ ID NO:21), GSTSGSGKSSEGKG (SEQ ID NO:22), GSTSGSGKSSEGSGSTKG (SEQ ID NO:23), GSTSGSGKPGSGEGSTKG (SEQ ID NO:24), or EGKSSGSGSESKEF (SEQ ID NO:25). Peptide or polypeptide linking moieties are described, for example, in Huston et al., Proc. Nat'l Acad. Sci. 85:5879, 1988; Whitlow et al., Protein Engineering 6:989, 1993; and Newton et al., Biochemistry 35:545, 1996. Other suitable peptide or polypeptide linkers are those described in U.S. Pat. Nos. 4,751,180 and 4,935,233, which are hereby incorporated by reference.

The peptides/peptidomimetics of the disclosure can comprise unnatural amino acids. The presence of unnatural amino acids can contribute to extended half-lives due to the reduction in proteolytic action. Unnatural amino acids have side chains or other structures not present in the 20 naturally-occurring amino acids listed above and include, but are not limited to: N-methyl amino acids, N-alkyl amino acids, alpha, alpha substituted amino acids, beta-amino acids, alpha-hydroxy amino acids, D-amino acids, and other unnatural amino acids known in the art (See, e.g., Josephson et al., (2005) J. Am. Chem. Soc. 127: 11727-11735; Forster, A. C. et al. (2003) Proc. Natl. Acad. Sci. USA 100: 6353-6357; Subtelny et al., (2008) J. Am. Chem. Soc. 130: 6131-6136; Hartman, M. C. T. et al. (2007) PLoS ONE 2:e972; and Hartman et al., (2006) Proc. Natl. Acad. Sci. USA 103:4356-4361).

Essentially any amino acid that, when attached to an appropriate tRNA, can be assembled into a polymer by natural or mutant ribosomes can be used (see Sando, S. et al., (2007) J. Am. Chem. Soc. 129:6180-6186; Dedkova, L. et al. (2003) J. Am. Chem. Soc. 125: 6616-6617; Josephson, K., Hartman, M. C. T., and Szostak, J. W. (2005) J. Am. Chem. Soc. 127:11727-11735; Forster, A. C. et al. (2003) Proc. Natl. Acad. Sci. USA 100:6353-6357; Subtelny, A. O., Hartman, M. C. T., and Szostak, J. W. (2008) J. Am. Chem. Soc. 130:6131-6136; and Hartman, M. C. T. et al. (2007) PLoS ONE 2:e972).

When unnatural amino acids are desired, it may be advantageous to use a purified translation system that lacks endogenous aminoacylated tRNAs (Shimizu, Y. et al. (2001) Nat. Biotech. 19:751-755; Josephson, K., Hartman, M. C. T., and Szostak, J. W. (2005) J. Am. Chem. Soc. 127: 11727-11735; Forster, A. C. et al. (2003) Proc. Natl. Acad. Sci. USA 100: 6353-6357). If unnatural amino acids are used with an in vitro translation system based on a lysate or extract, it may be desirable to deplete the extract of endogenous tRNAs, as previously described (see Jackson, R. J., Napthine, S., and Brierley, I. (2001) RNA 7:765-773). A system based on purified E. coli translation factors is commercially available (PURExpress™; New England Biolabs, Ipswich, Mass.). These systems are particularly useful for translation with unnatural amino acids to produce peptidomimetics.

When using natural amino acids with an in vitro translation system based on a lysate or extract, translation is dependent on the enzymatic charging of amino acids onto tRNAs by tRNA synthetases, all of which are components of the extracts. Alternatively, in vitro translation systems that use purified translation factors and ribosomes, or tRNA-depleted extracts, require that aminoacylated tRNAs be provided. In these instances, purified or in vitro synthesized tRNAs can be charged with amino acids using chemical (see Frankel, A., Millward, S. W., and Roberts, R. W. (2003) Chem. Biol. 10:1043-1050) or enzymatic procedures (Josephson, K., Hartman, M. C. T., and Szostak, J. W. (2005) J. Am. Chem. Soc. 127: 11727-11735; Murakami, H. et al. (2006) Nat. Methods 3:357-359).

After in vitro translation and/or isolation of peptide/peptidomimetics, the peptide moiety may be modified by intramolecular or intermolecular cross-linking, chemical conjugation, enzymatic cleavage, truncation, or extension with additional amino acid monomers. One way to accomplish this is by incorporating unnatural amino acids with reactive side chains into the polypeptides that make up the library. After translation, the newly formed polypeptides can be reacted with molecules that react specifically with the reactive side chain of the incorporated amino acid. For example, an amino acid with a terminal alkyne side chain can be incorporated into the polypeptide library and subsequently reacted with an azido sugar, creating a library of displayed polypeptides with sugars attached at the positions of the alkynyl side chains (Josephson, K., Hartman, M. C. T., and Szostak, J. W. (2005) J. Am. Chem. Soc. 127: 11727-11735). A variety of reactive side chains can be used for such post-translational conjugation, including amines, carboxyl groups, azides, terminal alkynes, alkenes, and thiols.

One particularly useful modification is based on the cross-linking of amino acids to produce cyclic structures. Cyclic regions in a protein contain a rigid domain, which reduces conformational flexibility and degrees of rotational freedom, leading to very high affinity binding to target proteins. A number of methods for cyclizing a polypeptide are available to those skilled in the art and are incorporated herein by reference. Typically, the chemical reactivity of specific amino acid side chains and/or the carboxyl or amino termini of the polypeptide are exploited to crosslink two sites of the polypeptide to produce a cyclic molecule. In one method, the thiol groups of two cysteine residues are cross-linked by reaction with dibromoxylene (see Timmerman, P. et al., (2005) ChemBioChem 6:821-824). Tri- and tetrabromoxylene can be used to produce polypeptides with two and three loops, respectively.

In another exemplary method, a side chain amino group and a terminal amino group are cross-linked with disuccinimidyl glutarate (see Millward, S. W. et al., J. Am. Chem. Soc. 127:14142-14143, 2005). In other approaches, cyclization is accomplished by making a thioether bridging group between two sites on the polypeptide (see Timmerman, P. et al., (2005) ChemBioChem 6:821-824; incorporated by reference herein in its entirety). One chemical method relies on the incorporation of an N-chloroacetyl modified amino acid at the N-terminus of the polypeptide, followed by spontaneous reaction with the thiol side chain of an internal cysteine residue (see Goto, Y. et al. (2008) ACS Chem. Biol. 3:120-129). An enzymatic method relies on the reaction between (1) a cysteine and (2) a dehydroalanine or dehydrobutyrine group, catalyzed by a lantibiotic synthetase, to create the thioether bridging group (see Levengood, M. R. and Van der Donk, W. A., Bioorg. and Med. Chem. Lett. 18:3025-3028, 2008). The dehydro functional group can also be generated chemically by the oxidation of selenium containing amino acid side chains incorporated during translation (see Seebeck, F. P. and Szostak, J. W. J. Am. Chem. Soc. 2006).

Once a single peptide or a pool of candidate peptide molecules is identified, they may undergo one or more rounds of structure activity relationship (SAR) optimization using standard chemical and peptide synthesis techniques. Such optimization may include considerations such as avoiding charged polar side chains (Asp, Glu, Arg, Lys) that may inhibit cell penetration, avoidance of side chains that pose metabolic liabilities (Tyr, Met, Trp, Cys), improving solubility, avoidance of unnecessary molecular weight, avoidance of rotatable bonds, and lipophilicity.

The amino acid sequences of the peptides/peptidomimetics of the disclosure may comprise only naturally occurring amino acids and as such may be considered to be peptides, polypeptides, or fragments thereof. Alternatively, the peptides may comprise both naturally and non-naturally occurring or modified amino acids or be exclusively comprised of non-naturally occurring amino acids. According to the disclosure, the compositions identified may be “peptide mimetics,” “peptidomimetics,” “peptides,” “polypeptides,” or “proteins.” While it is known in the art that these terms imply relative size, these terms as used herein should not be considered limiting with respect to the size of the various peptidic based molecules referred to herein and which are encompassed within this disclosure, unless otherwise noted.

According to the disclosure, any amino acid based molecule may be termed a “polypeptide” and this term embraces both “peptides” and “proteins.” Peptides are also a category of proteins and are traditionally considered to range in size from about 4 to about 50 amino acids. Dipeptides, those having two amino acid residues are a category of peptide as are tripeptides (3 amino acids). Polypeptides larger than about 50 amino acids are generally termed “polypeptide” or “proteins.” Peptide, polypeptide and/or proteins sequences may be linear or cyclic. For example, a cyclic peptide can be prepared or may result from the formation of disulfide bridges between two cysteine residues in a sequence. A peptide can be linked through the carboxy terminus, the amino terminus, or through any other convenient point of attachment, such as, for example, through the sulfur of a cysteine or any side-chain of an amino acid residue or other linkage including, but not limited to, a maleimide linkage, an amide linkage, an ester linkage, an ether linkage, a thiol ether linkage, a hydrazone linkage, or an acetamide linkage.

The disclosure contemplates several types of composition that are amino acid based including variants and derivatives. These include substitutional, insertional, deletion and covalent variants and derivatives. The term “derivative” is used synonymously with the term “variant” and refers to a molecule that has been modified or changed in any way relative to a reference molecule or starting molecule.

As such, included within the scope of this disclosure are polypeptide based molecules containing substitutions, insertions and/or additions, deletions and covalently modifications. For example, sequence tags or amino acids, such as one or more lysines, can be added to the peptide sequences of the disclosure (e.g., at the N-terminal or C-terminal ends). Sequence tags can be used for peptide purification or localization. Lysines can be used to increase peptide solubility or to allow for site specific modifications, such as, but not limited to, biotinylation or PEGylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence.

The disclosure provides that compounds disclosed herein can have prodrug forms. Prodrugs of the compounds are useful in the methods of this disclosure. Any compound that will be converted in vivo to provide a biologically, pharmaceutically or therapeutically active form of a compound of the disclosure is a prodrug. Various examples and forms of prodrugs are well known in the art. Examples of prodrugs are found, inter alia, in Design of Prodrugs, edited by H. Bundgaard, (Elsevier, 1985), Methods in Enzymology, Vol. 42, at pp. 309-396, edited by K. Widder, et al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p.1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392).

Prodrugs of compounds disclosed herein can be prepared by methods known to one of skill in the art and routine modifications thereof, and/or procedures found in U.S. Pat. No. 8,293,786, and references cited therein and routine modifications made thereof.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy to administer by a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound, e.g. a compound disclosed herein, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient plus any additional desired ingredient from a previously sterile-filtered solution thereof.

In a particular embodiment, one or more compounds of the disclosure are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to cells with monoclonal antibodies) can also be used as pharmaceutically acceptable carriers. These can be prepared according to methods known to those skilled in the art, for example, as described in U.S. Pat. No. 4,522,811.

The data obtained from the cell culture assays and animal studies can be used in formulating a range of dosage for use in humans. The dosage of such compounds lies within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma can be measured, for example, by high performance liquid chromatography.

Compositions and formulations of one or more compounds disclosed herein can be used in combination with other active agents to treat a disorder or disease in a subject.

It should be understood that the administration of an additional therapeutic agent with a compound of the disclosure encompasses co-administration of these therapeutic agents in a substantially simultaneous manner, such as in a single capsule having a fixed ratio of active ingredients or in multiple, separate capsules for each active ingredient. In addition, administration of an additional therapeutic agent in combination with a compound disclosed herein also encompasses use of each type of therapeutic agent in a sequential manner. In either case, the treatment regimen will provide beneficial effects of the drug combination in treating the disorders described herein.

In a further embodiment, the compounds disclosed herein can be combined with one or more class of therapeutic agents, including, but not limited to, alkylating agents, cancer immunotherapy monoclonal antibodies, anti-metabolites, mitotic inhibitors, anti-tumor antibiotics, topoisomerase inhibitors, photosensitizers, tyrosine kinase inhibitors, anti-cancer agents, chemotherapeutic agents, anti-migraine treatments, anti-tussives, mucolytics, decongestants, anti-allergic non-steroidals, expectorants, anti-histamine treatments, anti-retroviral agents, CYP3A inhibitors, CYP3A inducers, protease inhibitors, adrenergic agonists, anti-cholinergics, mast cell stabilizers, xanthines, leukotriene antagonists, glucocorticoid treatments, antibacterial agents, antifungal agents, sepsis treatments, steroidals, local or general anesthetics, NSAIDS, NRIs, DARIs, SNRIs, sedatives, NDRIs, SNDRIs, monoamine oxidase inhibitors, hypothalamic phoshpholipids, anti-emetics, ECE inhibitors, opioids, thromboxane receptor antagonists, potassium channel openers, thrombin inhibitors, growth factor inhibitors, anti-platelet agents, P2Y(AC) antagonists, anti-coagulants, low molecular weight heparins, Factor VIa inhibitors, Factor Xa inhibitors, renin inhibitors, NEP inhibitors, vasopepsidase inhibitors, squalene synthetase inhibitors, anti-atherosclerotic agents, MTP inhibitors, calcium channel blockers, potassium channel activators, alpha-muscarinic agents, beta-muscarinic agents, anti-arrhythmic agents, diuretics, thrombolytic agents, anti-diabetic agents, mineralocorticoid receptor antagonists, growth hormone secretagogues, aP2 inhibitors, phophodiesterase inhibitors, anti-inflammatories, anti-proliferatives, antibiotics, farnesyl-protein transferase inhibitors, hormonal agents, plant-derived products, epipodophyllotoxins, taxanes, prenyl-protein transferase inhibitors, anti-TNF antibodies and soluble TNF receptors, Cyclooxygenase-2 inhibitors, and miscellaneous agents.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds or agents described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

Via genetic and pharmacological experiments in vitro and in vivo, the disclosure provides, for the first time, a novel signaling Y814 residue within gp130 module that represents an initiating factor in upregulation of inflammatory and catabolic pathways including activation of SRC family kinases along with NF-κB transcription factor, which have been nominated as regulators in degeneration and chronic inflammation in inflammaging, arthritis and other diseases. The disclosure demonstrates that transgenic mice with constitutively inactivated Y814 (F814) are viable and fertile as homozygotes; they show no obvious phenotype in musculoskeletal tissues in homeostatic conditions. Moreover, all tested cell types from F814-CRISPR mutant mice show atypical responses to IL-6 cytokines with drastically reduced activation of SRC kinase and cartilage matrix degeneration in response to gp130 stimulation. Further, the disclosure demonstrates that a newly developed polyclonal antibody against pY814 confirmed marked activation of this residue in response to IL-6 cytokines in wild type, but not in F814 mutant mice. The studies show that Y814 residue can be inhibited by a small molecule with a significant reduction in pain levels and cartilage degeneration in canine model of post-traumatic OA. Altogether those findings demonstrate a novel and clinically relevant signaling module. The disclosure demonstrates that this gp130 tyrosine is a potential novel therapeutic target for OA. Consequently, gp130 Y814 is a mediator of inflammaging and tissue degeneration directly implicated in the pathogenesis of OA.

As a tool to elucidate the role of this gp130 residue, the disclosure provides a CRISPR mouse with a specific point mutation at residue Y814 (Y812). This mouse represents a paradigm-shifting tool in inflammation related research. It can be commercialized as a research tool to study the biological effect of this tyrosine on inflammatory activity. Using the mouse model and the discovery of the biological activity of Y814 (Y812), the disclosure provides methods to identify small molecules and biologics to target this residue and specifically modulate interaction between this residue and downstream partners activated via IL6 cytokines via Y814 (Y812).

The disclosure also provide an antibody that can be used as a biomarker-agent as a predictive sign of prognosis for various diseases such as cancer and autoimmune/inflammatory diseases. In addition, by using the commercially produced Y814 antibody as a detection method, a high throughput small molecule screen can be performed for innovative pharmaceutical/pharmacological drug therapy. The screening will allow one to identify small molecules that may activate/deactivate Y814 residue and indicate various therapeutic effects of this activation/deactivation in inflammatory disorders, cancer and age associated degeneration.

The compositions and methods of the disclosure provide for diagnostics and therapeutics for the treatment of a variety of clinical indications including cancer, autoimmunity/inflammation.

The invention is illustrated in the following examples, which are provided by way of illustration and are not intended to be limiting.

### EXAMPLE

### Example 1

Experimental procedures: First, the molecular pathways and potential mediators implicated in downstream signaling of gp130 Y814 activated by IL-6 family cytokines in different cell types will be defined. SRC kinase family consists of 9 members: SRC, YES, FYN, FGR, LCK, HCK, BLK, LYN and FRK, and, via RNA-seq, that some, albeit not all, kinases are expressed in the adult synovial joint. In order to determine which IL-6 cytokines require Y814, various cell types derived from WT and F814 mice will be used to not only confirm the necessity of the residue, but also to investigate if this mechanism is cell-type specific. Vascular cells and macrophages play a critical role in the chronic inflammatory process and it remains to be determined if Y814 residue activates SRC kinases and their targets, including NF-κB, in each of these cell types. Chondrocytes will be derived from the anatomically defined femoral head cartilage of 1-week old pups.

For cell isolation, cartilage tissue will be digested. From the same animals, pericytes (CD146+CD31−CD45−), endothelial cells (CD31+CD45−) and macrophages (CD11b+CD45+) from the synovial tissue and bone marrow will be sorted, respectively. To assess the differences in gp130 Y814 levels downstream of various IL-6 family cytokines, cells will be stimulated with OSM, LIF, IL-6, CNTF, IL-11 and hyper-IL-6 for 24 hours followed by Western and ICC using pY814, pSRC and pNF-κB, pERK1/2, pSTAT3 antibodies to determine their respective levels in the WT vs. mutant chondrocytes, pericytes, endothelial cells and macrophages. Initial studies have shown that SHP2/ERK and JAK/STAT signaling is minimally affected by the Y814 mutation.

Next, experiments are performed to determine which SRC kinases are dependent on Y814 in mouse chondrocytes. Certain SFKs are not expressed in all tissues. SRC, FYN, YES and YRK are ubiquitously expressed whereas HCK (myeloid cells), FGR (myeloid cells, B cells) and LYN (brain, B cells, myeloid cells) tend to be more restricted to blood cells. Currently, experiments have only focused on SRC (c-SRC) and its activation by OSM in pig, human and mouse chondrocytes. Even though members of the family have a conserved domain organization (a myristoylated N-terminal segment, an SH3, SH2 and tyrosine kinase domains, and a C-terminal tail), they nevertheless possess a unique domain that exhibits a strong sequence divergence among the family members which may contribute to differences in signaling and lead to distinct regulation. To demonstrate which SFKs are affected by Y814, WT and F814 1-week old mouse femoral head-derived and adult sternal chondrocytes will be stimulated with IL-6 cytokines and hyper-IL-6 for 24 hours followed by a Western blot using antibodies for all 9 SFKs to determine kinase activation profiles. IHC staining of WT and F814 mouse joint sections will also be performed as an additional confirmation of SFK expression.

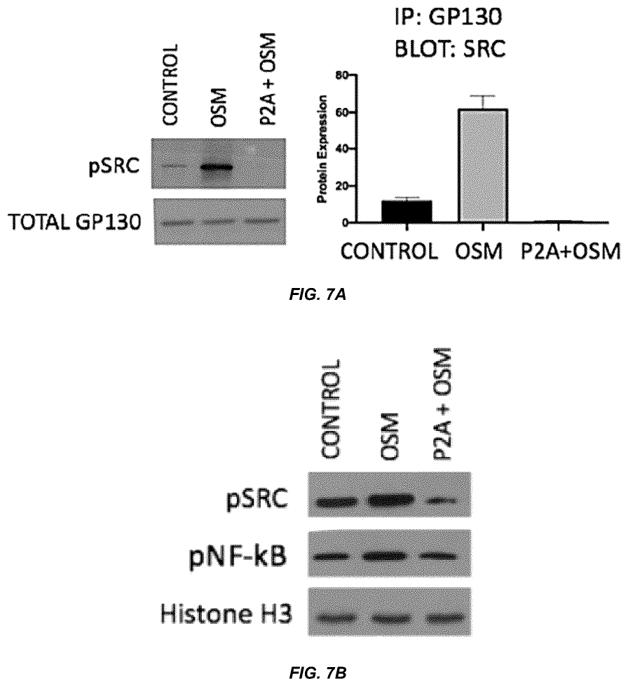

In prostate cancer cell lines, SRC mediates scatter factor cytokine stimulation of NF-κB activity via TAK1/NIK/NF-κB pathway shows that SRC plays a role in TAK1 and NF-κB signaling. In some cell types, TAK1 is essential for NF-κB activation and is a central component of NF-κB signaling. The initial data have demonstrated that IL-6 cytokines can dramatically upregulate TAK1 in WT spleen cells, but this response is almost absent in Y814 deficient cells nominating TAK1 as one of the key players downstream of Y814 (FIG. 7A). Currently, it is unclear whether SRC-dependent NF-κB activation is mediated by TAK1 even though previous studies demonstrated that SRC can directly interact with NF-κB IKK complex. In order to elucidate the definitive role of TAK1 as a mediator of SRC and NF-κB in a gp130 Y814-dependent mechanism, WT and F814 mouse cell types noted above will be stimulated with IL-6 cytokines and hyper-IL-6 for 24 hours followed by Western and ICC detecting TAK1 protein activation by pTAK1 antibody. If TAK1 is a critical mediator in gp130 Y814-dependent signaling, TAK1 expression should be inhibited in the mutant cells correlating to decreased levels of pSRC and pNF-κB. SRC inhibition can prevent matrix catabolism downstream of OSM, and that in F814 mouse knee cartilage explants cartilage degeneration is significantly decreased relative to WT as shown by low matrix neoepitope generation in the mutant (FIG. 7B).

In order to assess the distinct functional significance of Y814 residue, WT and F814 1-week old mouse femoral head-derived chondrocytes and pig chondrocytes will be used to conduct functional assays since cartilage is the major target in OA. Legs from 5 five-month-old Yucatan minipigs will be obtained from S&S Farms, and cartilage will be harvested and digested. The WT and mutant chondrocytes will be stimulated with IL-6 cytokines and hyper-IL-6 for 24 hours to induce catabolism, and pig chondrocytes will be treated with SRC, NF-κB and TAK1 inhibitors for 24 hours prior to harvesting. Cell viability, proliferation (EdU assay), apoptosis (Annexin V and TUNEL assays), matrix catabolism (qPCR and COL2 and aggrecan neoepitope assay and biosynthesis (COL2A1, aggrecan expression and extracellular matrix domain (EMD) assay and cell migration (wound healing assay) will be measured to determine the effect of the Y814 mutation and SRC, NF-κB and TAK1 inhibition on functional outcomes.

Outcomes: The data will show that gp130 Y814 residue is essential for downstream activation of SRC TAK1-NF-κB axis and is cell-type specific, and that mutation of this residue will markedly prevent matrix catabolism and degeneration in vitro. In addition, the data will show that SFKs are differentially dependent on Y814 and not all will require activation of this residue.

A gp130-SRC-YAP module has been described; thus, it cannot be excluded that YAP may play a role in gp130-dependent matrix catabolism and cartilage degeneration. Inhibition of YAP prevents cartilage degradation and ameliorates OA; however, opposing data has demonstrated that YAP is highly upregulated in human and mice OA cartilage. Therefore, the role of YAP in cartilage degradation remains elusive. Previously, YAP has been shown to directly interact with TAK1 and mitigate NF-κB signaling by inhibiting substrate accessibility of TAK1 in chondrocytes. Accordingly, if the studies fail to define TAK1 as an mediator of gp130 Y814-SRC-NF-κB signaling, experiments will be performed to assess the role of YAP in this axis. Western blots and Cut & Run sequencing will be performed utilizing YAP antibody to detect protein levels and transcription of YAP, respectively, in the WT vs. Y814 mutant mouse. To delineate if YAP/TAK1 interaction is present in WT and Y814 mutant chondrocytes derived from 1-week old femoral heads, co-immunoprecipitation capturing YAP immune complexes and blotting for TAK1 antibody; detection of TAK1 will confirm protein-protein interactions.

Determine the functional outcomes of a novel SRC-targeting peptide 2A (P2A). Rationale: Synthetic peptides mimic a protein of interest, potentially hindering its interaction with other proteins; peptides that interfere with protein-protein interactions are termed interfering peptides. This is favorable for controlled interference of specific factors directly involved in intramolecular interactions with receptors. Peptides are specific to the protein of interest and mimic natural endogenous motifs resulting in minimal side effects in vivo. The advantages of synthetic peptides is that they are small (less than 55 amino acids in length) (61), are easily synthesized, can penetrate cell membranes and do not accumulate in organs, minimizing toxicity (62).

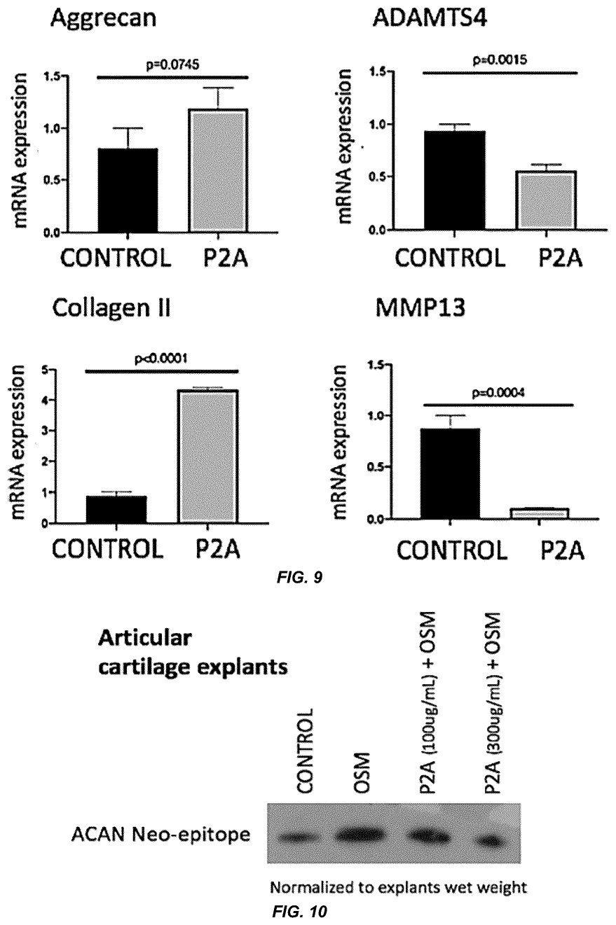

The studies delineate that gp130 Y814 regulates SRC-NF-κB downstream signaling; since SRC is known to associate with gp130, it was hypothesized that gp130 and SRC are capable of physically interacting on Y814. In order to impede this interaction, a screen of short and overlapping peptides mimicking the motif of gp130 around Y814 that were individually tested for SRC and NF-κB inhibition were tested. Out of all the synthesized peptides, a 4 amino acid interfering peptide motif was identified that is termed peptide 2A (P2A) and which was the most efficacious at physically hindering gp130-SRC protein-protein interaction by binding to SRC (c-SRC), preventing accessibility to Y814 as an active site (FIG. 8A) and decreasing SRC and NF-κB activation (FIG. 8B). Due to sequence specificity, the peptide is capable of discriminating among other gp130 regions serving as a competitive inhibitor. Docking for peptide-protein interaction interfaces confirmed that P2A has a strong binding affinity to c-SRC as shown by the spatial proximity through protein folding in resolved 3D structure. It is important to note that since SRC interacts with or is activated by various other receptors such as integrins, EGFR, TNFR, HGFR, IGF-1R, PDGFR and other RTKs (63-66), complete inhibition of SRC activity may prevent signaling of these receptors through SRC which may be detrimental; specificity of P2A bypasses the potential deleterious effect of total SRC inhibition. However, it is unknown whether interaction between other SFK members will replicate the interaction detected with SRC. P2A decreased transcription of catabolic enzymes (ADAMTS4/5 and MMP13), promoted biosynthesis (COL2 and ACAN) in OA chondrocytes (FIG. 9) and prevented matrix degeneration in pig cartilage explants induced by OSM (FIG. 10). Based on the preliminary data indicating that P2A is capable of interfering with SRC-NF-κB signaling, it was hypothesized that administration of P2A will block tissue degenerating pro-inflammatory milieu in vivo and in vitro and that individual SFKs will have different binding affinities to P2A.

In order to elucidate the role of P2A in vitro, pericytes (CD146+CD31−CD45−), endothelial cells (CD31+CD45−), and monocytes (CD11b+) from normal (healthy) and OA human synovium via flow cytometry, will be isolated and obtained. Concurrently, articular chondrocytes from normal and OA patients (see above) will be used.

Cells isolated from normal patients will be stimulated with IL-6 cytokines and hyper-IL-6 with and without treatment of P2A for 24 hours at the dose of 300 μg, while OA cells will only be treated with and without P2A for 24 hours at the same dose. OA cartilage and synovium endogenously have an excess of pro-inflammatory cytokines and a deficit of anti-inflammatory cytokines leading to persistent tissue destruction; therefore, no cytokine treatment will be necessary. The initial studies demonstrated that 300 μg of P2A is a useful dose for sustained prevention of catabolism in pig cartilage (FIG. 10) and that P2A had a significant effect on decreasing SRC and NF-κB signaling in OSM stimulated pig chondrocytes.

Therefore, the molecular pathways and mediators downstream of gp130 that are affected by administration of P2A in different human cell types will be determined. All P2A-treated stimulated and unstimulated cells (chondrocytes, pericytes, endothelial cells and monocytes) from normal adult and OA patients, respectively, will be subjected to Western blot to determine the protein levels of pSFKs, pNF-κB, and pTAK1. In addition, the co-IP data demonstrated that P2A interacts with SRC (c-SRC); in order to determine if P2A is capable of binding to other SFKs, all cells will be subjected to co-IP, immunoprecipitated with gp130 as a complex and blotted with various antibodies for all SFK family members. As an additional readout in pericytes, chondrocytes, endothelial cells and monocytes, secretion of pro-inflammatory (TNF-α, IL-1, IL-6, IL-17), and anti-inflammatory (IL-4, IL-10, IL-27 cytokines) by ELISA (RayBiotech) will be measured. Likewise, a human cytokine protein array (RayBiotech C2000 array) will be analyzed to objectively detect various cytokines. These experiments will provide a direct measure of the change of the inflammatory microenvironment following treatment of P2A.

Initial studies suggest that P2A prevents degeneration in pig cartilage explants. To assess the effects of P2A on normal and OA cartilage, a neo-epitope assay will be performed on human cartilage explants and quantify the expression of catabolic (ADAMTS4/5, MMP13) and anabolic genes (COL2A1, ACAN) by qPCR on chondrocytes; biosynthesis via EMD assay will also be determined. In addition, cell viability, proliferation (EdU assay), apoptosis (Annexin V and TUNEL assays) and cell migration (wound healing assay) will be performed.

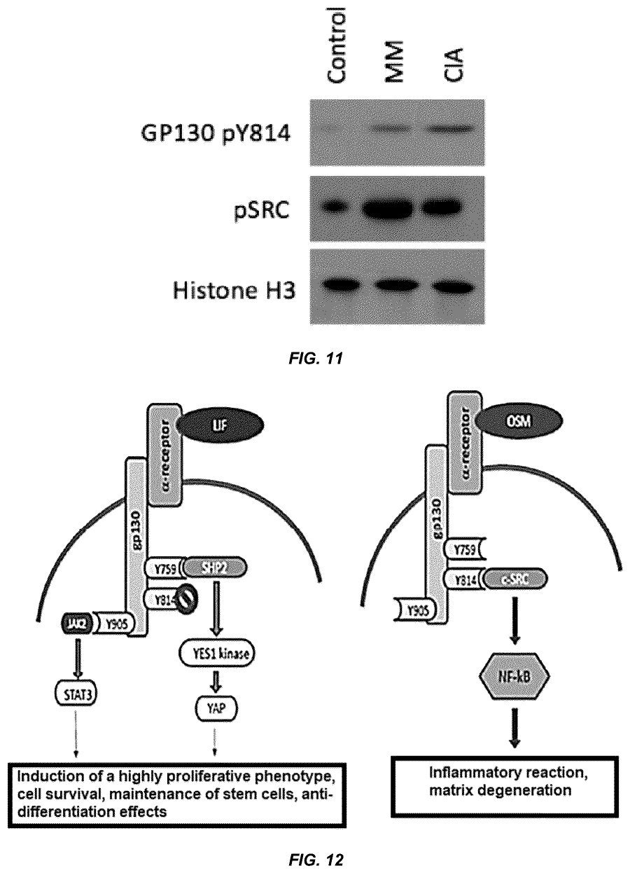

To define the role of P2A in disease progression in vivo, two mouse models of arthritis, a medial meniscectomy (MMJ) model for post-traumatic OA and a collagen-induced arthritis (CIA) model for inflammatory arthritis will be employed. Initial studies showed that in MM and CIA models, gp130 Y814 and pSRC are highly upregulated relative to sham animals (FIG. 11). P2A and control peptide will be injected intraarticularly into experimental and sham animals every week, starting one week after induction of disease at dose 300 mg/mL; this dose showed efficacy in vitro.

In the MM model, at least 11 males each will be used for sham, control and experimental groups to statistically establish the effects of P2A injection. Males are used due to fewer confounding variables in this injury-induced model. All analyses will be performed at 8 weeks post experimental or sham surgery (joint opening but no meniscectomy). The animals of each group will be examined using immunostaining and Safranin O staining (detecting proteoglycans) assessing morphology and matrix deposition in growth plates and articular cartilage coupled with OASRI scoring. Immunostaining and in situ hybridization (ISH) for markers characteristic of distinct growth plate zones (COL2A1: columnar; IHH: late columnar/prehypertrophic; COL10A1: prehypertrophic/hypertrophic; OPN: late hypertrophic) will detect differences in onset, pace, and extent of maturation. Synovitis will be evaluated. Defects in proliferation and survival will be documented by PCNA and TUNEL. Levels and location of pSRC, pTAK1, and pNF-κB will be examined using commercial antibodies. Additionally, synovium and chondrocytes will be isolated from 3 mice in each group and perform total joint scRNA-Seq. The analysis of this data will focus on genes affiliated with OA, e.g., Adamts4/5, MMPs and osteoclast genes and their relationships with gp130. These data sets will be overlapped with published data using GSEA to identify unique pathways that are enriched in an OA-like model and/or may prevent progression. Treatment with P2A will result in a protective, anti-degenerative effect on articular cartilage and synovium.

In this model, mice will be anesthetized and medial para-patellar arthrotomy is carried out under a dissection microscope; the knee will be flexed to expose the meniscus. Half of the meniscus will be removed and the joint closed. Animals that have their joints exposed but do not undergo partial meniscectomy will be used as sham controls. Eight weeks after surgery, animals will be sacrificed and joints embedded in paraffin. OARSI histological scoring and immunohistochemical detection of matrix proteins will be performed as well as the markers of viability, catabolism, and chondrocyte hypertrophy of articular cartilage.

IHC and standard image quantification techniques for COL1A1 and COL2A1 will be used to evaluate the nature of cartilage matrix the defects in control and P2A-treated animals, while TUNEL will define apoptotic rates of articular chondrocytes. Indian Hedgehog, RUNX2 and COL10A1 will be used as markers of hypertrophy, while MMP13, ADAMTS4/5, pNF-κB, pSRC, pTAK1 and collagen/aggrecan neo-epitopes will indicate matrix degeneration. Finally, to assess the dependence of joint function following injury on gp130, behavioral testing will be performed as a surrogate for pain. Equipment to assess general activity (wheel run assay; WRA) and gait (dynamic weight bearing; DWB) will be used. The DWB assay enables quantitative analysis of usage of individual paws during normal activity including walking, rearing and sitting, thus defining any potential bias due to pain. Animals will be assessed before surgery and at 2-week intervals during the course of the experiment; each will serve as its own control. In composite, the structural and behavioral results will be compared among animals of all genotypes to specify how P2A treatment contributes to disease progression.

To quantify the signaling milieu in the joint, several additional analyses will be performed in addition to those already detailed above. At the time of sacrifice, synovial fluid will be harvested from all 11 mice in each group and pooled from 3-4 animals. Levels of 144 proteins will be semi-quantitatively determined using arrays from RayBiotech (C2000 array), including the anabolic and anti-inflammatory factors FGF2, IGF1/2, IL-4, IL-10, IL-1RL1, IL-27, Shh and TIMP1/2 and the pro-degenerative/pro-inflammatory factors IL-6, IL-6R, IL-1A, CXCL8 (IL-8), IL-17, MMP-2/3/9 and TNF-α. These arrays will provide a direct measure of the change in the injured joint microenvironment following manipulation of gp130 signaling.

In parallel, similar experiments will be performed in the CIA model in which cartilage degradation is mediated by a very active inflammatory response that could potentially be counteracted by administration of P2A. To elicit this response, mice are co-injected with Complete Freund's Adjuvant (CFA) and bovine collagen II. CFA contains heat killed mycobacterium which activate the innate immune system, while the collagen II provides an antigen for the adaptive immune response. This protocol results in immune cell infiltration, synovitis, pannus formation and articular cartilage degeneration in a variable number of both small and large joints. For these experiments, both male and female mice will be used (n=11 in each group). An emulsion of collagen II and CFA will be injected subcutaneously at the base of the tail. Three weeks later, a booster injection of the same solution will be administered; after an additional 3 weeks allowing for maximal response, animals will be subjected to final DWB and WRA behavioral analysis, sacrificed and affected small and large joints harvested for molecular and structural assessment as above.

Outcomes: Treatment with P2A will impede an inflammatory microenvironment in vitro by physically hindering activation of SRC downstream signaling and modulate the secretion of detrimental pro-inflammatory cytokines in normal tissue. The endogenous inflammatory milieu in OA tissue will be reduced via downregulation of gp130-SRC-NF-κB signaling and decline of pro-inflammatory cytokines. In addition, the administration of P2A will prevent joint destruction in the in vivo MM and CIA mouse models. P2A will serve as a potential translational tool to modulate SRC-mediated pathogenesis with high specificity due to limited inhibitory profile.

A cell penetrating peptide (CPP) delivery system will be used in the experiments of the disclosure; CPPs have been shown to promote the delivery of peptides effectively into live cells. A CPP will be conjugated to P2A to increase cell permeability and facilitate uptake. Additional structural modifications can be made to the peptide to improve its biological activity and stability. Unmodified native peptides may have a short half-life, solubility and bioavailability; introduction of modified amino acids can represent a solution in this eventuality. In addition, to increase protease resistance, a cyclized P2A will be generated that will generate a bond between the original N- and C-termini of P2A. Alternatively, PEGylation can be used to increase the half-life by conjugating polyethylene glycol to the peptide, which will reduce renal filtration. In addition, microsomal or nanoparticle-based delivery systems may be employed should optimization of pharmacokinetics be necessary.

It has been demonstrated that IL-6 family cytokines that signal through gp130 can promote arthritic outcomes in animal models, including inflammation and cartilage degradation. However, it is not known to what extent these pro-arthritic effects of gp130 and IL-6 cytokines are dependent on Y814 of gp130. The data suggest that activation of this residue in multiple cell types is primarily responsible for gp130-mediated activation of NF-κB and the downstream destructive effects. Interestingly, different IL-6 cytokines evidence varying propensities to activate Y814; specifically, OSM>LIF (FIG. 6). This is the converse of these cytokines' capacity to promote tissue regeneration and stem/progenitor cell activity. Accordingly, it is hypothesized that activation of Y814 shunts activity downstream of gp130 away from a pro-regenerative signaling milieu toward a pro-inflammatory state. Furthermore, that mutation of Y814 will not only reduce joint damage following injury or acute inflammation but shift the joint microenvironment toward a pro-regenerative state.

Moreover, there have been few efforts to address which cell types may require gp130 signaling to promote joint disease or maintenance; it is worth noting that the data demonstrate that gp130 signaling in chondrocytes is beneficial during homeostasis, promoting chondrocyte proliferation and growth plate integrity. Several candidate cell types that may drive inflammation and cartilage catabolism downstream of gp130 include phagocytes (macrophages and neutrophils), endothelial cells and perivascular cells. Macrophages and neutrophils are known “cytokine factories” that respond to tissue injury, with neutrophils acting as first responders to tissue damage to be followed by monocyte-derived macrophages. Both of these cell types shed IL-6R locally as well as producing IL-6, thus providing a potentially strong pro-inflammatory signal in the synovium. Endothelial cells respond to IL-6 by producing chemotactic factors that recruit additional immune cells, thus behaving as cellular gatekeepers following injury. However, as they do not express IL-6R, endothelial cells require pre-formed IL-6/IL-6R complexes to activate gp130. Interestingly, deletion of gp130 in endothelial cells strongly disrupts extravasation by neutrophils; the effects of this cell-type specific deletion has not been evaluated in a mouse of arthritis. Directly adjacent to endothelial cells, pericytes also separate immune cells from circulation and the tissue parenchyma. The role of pericytes in mediating neuro-inflammation has been studied extensively, but their function in modulating immune cell infiltration and cartilage degeneration is not well documented. Accordingly, it is hypothesized that synoviocytes, including pericytes, endothelium and innate immune cells, function downstream Y814 activation to secrete pro-inflammatory and pro-degenerative factors that drive cartilage loss. It is also proposed that Y814-SRC signaling cascade in chondrocytes further enhances degenerative changes initiated by the stromal and immune cells in the joint.

Experimental procedures. In order to examine the role of Y814 in chondrocytes, phagocytes, endothelial cells and pericytes in promoting arthritic phenotypes following joint injury without the confounding effects of developmental phenotypes, gp130fl/fl mice will be generated that will permit conditional deletion in the appropriate cell type. Accordingly, Acan-CreERT2 (chondrocytes, present in Evseenko lab), LysM-CreERT2 (phagocytes; JAX 032291), Cdh5-CreERT2 (endothelium; Taconic 13073) and Pdgfrb-CreERT2 (pericytes; JAX 030201) mice and cross them with gp130fl/fl mice to generate animals hetero/hemizygous for CreERT2 and homozygous for gp130fl/fl. To create cell-type specific expression of solely F814, gp130fl/fl;CreERT2 animals will be crossed with gp130F814/F814 animals to generate gp130fl/F814;CreERT2 progeny, where CreERT2 represents any of the cell-specific deletion lines mentioned above (Acan/LysM/Cdh5/Pdgfrb-CreERT2). Administration of tamoxifen in these animals will result in excision of the WT gp130 allele (which is fully signaling competent before deletion) and continued expression of the F814 allele; this strategy has been applied previously with other gp130 mutants. Animals that are gp130fl/+;Cre+ generated in crosses of gp130fl/fl;CreERT2 animals with gp130+/+ mice will serve as controls.

Both the CIA and MM models described will be employed to assess the cell-type specific requirements for Y814 signaling in disease progression. In the MM model, tamoxifen will be injected into skeletally mature 4 month old gp130fl/F814;Acan-CreERT2, gp130fl/F814;LysM-CreERT2, gp130fl/F814;Cdh5-CreERT2 and gp130fl/F814;Pdgfrb-CreERT2; at least 11 males each will be used sham, control and experimental groups to statistically establish the effects of cell-type specific modifications on Y814 signaling on articular cartilage degeneration. Surgery will be performed 2 weeks after the final tamoxifen injection. Males are used due to fewer confounding variables in this injury-induced model. All analyses will be performed at 8 weeks post experimental or sham surgery.

11 animals of each experimental and control genotype will be examined using immunostaining and Safranin O staining (proteoglycans) coupled with OASRI scoring; 3 additional animals will undergo total joint scRNA-seq. It is expected that cell-type specific expression of solely F814 under homeostatic conditions will have little effect on articular cartilage and synovium due to the lack of phenotype in global homozygous F814 animals maintained without injury or inflammatory stimulus.

The impact of cell-type specific expression of solely Y814 on cartilage injury will then be tested. It is hypothesized that expression of solely F814 should impair disease progression in the MM model. To address the function of gp130 in chondrocytes, phagocytes, endothelium and pericytes in promoting OA-like symptoms, 11 gp130fl/F814;Acan-CreERT2, gp130fl/F814;LysM-CreERT2, gp130fl/F814;Cdh5-CreERT2 and gp130flF814l;Pdgfrb-CreERT2 males will be compared to gp130fl/+;Cre+ control and sham animals; 3 animals of each experimental, control and sham group will undergo total joint scRNA-seq. Surgeries will be performed 2 weeks after the final tamoxifen injection. Eight weeks after surgery, animals will be sacrificed and analyzed as in Aim 2. In composite, the structural, behavioral and molecular results will be compared among animals of all genotypes to specify how loss of Y814 signaling in individual cell types contributes to disease progression.

To assess the capacity of Y814 to suppress a proliferative and anabolic response, male gp130F814/F814 will be compared to control gp130fl/fl mice 8 weeks after MM surgery; sham groups for each genotype will also be analyzed. EdU will be injected IP daily for four days in weeks 7 and 8 to detect proliferation. Behavior and joints will be analyzed by histology and scRNA-seq as above. To quantify the signaling milieu in the joint, synovial fluid will be harvested from all 11 mice in each group and pooled from 3-4 animals. Levels of 144 proteins will be determined using arrays from RayBiotech. Proliferation will be assessed by co-staining for EdU and markers of chondrocytes, M1/M2 macrophages and pericytes. For these experiments, both male and female mice will be used (n=11 of each genotype). Two weeks after the final tamoxifen injection, animals will undergo induction; at 6 weeks post-induction, animals will be sacrificed and affected small and large joints embedded in paraffin for structural assessment or total joint scRNAseq as above. All animals expressing Cre (gp130fl/F814;Acan-CreERT2/LysM-CreERT2/Pdgfrb-CreERT2/Cdh5-CreERT2) will be compared to their sex-matched, gp130fl/+;Cre+ controls to allow for assessment of how the Y814 signaling module in specific cell types impacts progression of arthritic symptoms in an acute inflammatory model.

Outcomes: Based on initial data, most of the pro-inflammatory effects of gp130 are mediated by Y814. Consequently, the expression of only F814 will strongly reduce the pathogenic effects mediated by gp130 in both the MM and CIA models. Moreover, it is hypothesized that global expression of F814 will abrogate the feed-forward, pro-inflammatory loop downstream of IL-6 family cytokines that signal through gp130 and activate Y814. If these hypotheses prove accurate, then a new and functional biomarker that mediates the balance between de- and regeneration downstream of gp130 will be identified. These findings could support development of future therapeutics that specifically target Y814.

### Example 2

General methods. For all experiments, biological replicates (cells from independent specimens) were employed to generate data. For experiments expected to yield large differences, standard practice of using 3-5 replicates was followed.

Cell culture and treatments. Only early passages of chondrocytes (passage 0-2) were used for experimentation to avoid de-differentiation and loss of cartilage phenotype. Fetal tissue was provided by Dr. April Pyle (UCLA). Normal and OA adult articular cartilage were obtained from the National Disease Research Interchange.

Ba/F3 and ATDC5 cells were purchased from ATCC (Manassas, VA). Mouse spleen, fat and cartilage tissue were obtained from the wild-type and mutant F814 animals attained from the Jackson Laboratory and USC Transgenic Core, respectively. At termination, mouse tissues of interest were dissected, harvested and digested following previously established protocols. Legs from five-month-old Yucatan minipigs were obtained from S&S Farms (Ramona, California), and cartilage was harvested and digested following previously established protocols. Cartilage explants were made using 2 mm biopsy punch (Miltex, Inc., York, PA) and wet weight of each explant determined prior to experimentation. For cell isolation cartilage tissue was digested as described previously.

Cell culture reagents were purchased from Life Technologies, Inc. (Grand Island, New York). Growth factors LIF, OSM, IL-6, IL-11, CNTF and hyper-IL6 were purchased from Peprotech (Rocky Hill, NJ). SRC (SU6656) inhibitor was purchased from Selleckchem (Pittsburgh, PA). R805 was synthesized via a fee-for-service arrangement with Chares River, UK. Media was replenished with DMEM F12 medium containing 1% (vol/vol) fetal bovine serum and 1% Penicillin-Streptomycin (vol/vol) once treatments were added.

Statistical analysis. Numbers of repeats for each experiment are indicated in the figure legends. Pooled data are represented as mean±SD unless otherwise indicated. Unless otherwise indicated, statistical analysis was performed using one-way ANOVA followed by the Tukey test to compare more than 2 groups or 2-tailed Student's t test to compare 2 groups. p values less than 0.05 were considered to be significant.

IP Western, SDS-PAGE and Western blot analysis. For transfections of gp130 wild type (WT) (InVivoGen) and gp130 mutant plasmids (Thermo Scientific), cells were plated 24 h prior to transfection to produce monolayers that were 60% confluent and these were transfected by using Turbo DNAfectin 3000 (cat #G3000, Lambda Biotech) according to the manufacturer's protocol. 72h after transfection, cells were maintained in DMEM (10% FBS, 1% PSA) and were either left untreated or were stimulated with IL-6 (10 ng/mL) and/or OSM (10 ng/mL) for 24 h before cell harvest and protein extraction. For immunoprecipitation assays, cell lysates were incubated with Protein G Agarose (cat #20398, Pierce) and anti-Flag antibody (cat #2368, Cell Signaling) at 4° C. overnight. The immune complexes were sedimented, washed and separated by SDS-PAGE (see below) and further analyzed by Western blot using p-SRC (pY416-SRC) (cat #1246F, Novus Biologicals) or NEMO (cat #18474-1-AP, Proteintech) antibodies and normalized to gp130 (cat #bs-1459R, Bioss) or total SRC (cat #2123, Cell Signaling). For R805 treatment, the immune complexes were sedimented, washed and separated by SDS-PAGE and further analyzed by Western blot using OSMR (cat #ab85575, Abcam), LIFR (Santa cat #515337, Cruz Biotechnology) or p-SRC (pY416-SRC) (Novus Biologicals, cat #1246F). Histone H3 (cat #9715, Cell Signaling), gp130 (cat #bs-1459R, Bioss), total SRC (cat #2123, Cell Signaling) or Flag antibody (cat #14739, Cell Signaling) were used as loading controls.

For standard western blot, treated and non-treated cells were lysed in RIPA Lysis and Extraction Buffer (Pierce, Rockford, IL) containing protease inhibitors (Pierce) followed by sonication with a 15-second pulse at a power output of 2 using the VirSonic 100 (SP Industries Company, Warminster, PA). Protein concentrations were determined by BCA protein assay (Pierce). Proteins were resolved with SDS-PAGE utilizing 4-15% Mini-PROTEAN TGX Precast Gels and transferred to Trans-Blot Turbo Transfer Packs with a 0.2-μm pore-size nitrocellulose membrane. The SDS-PAGE running buffer, 4-15% Mini-PROTEAN TGX Precast Gels, Trans-Blot Turbo Transfer Packs with a 0.2-μm pore-size nitrocellulose membrane was purchased from Biorad (Hercules, CA). Nitrocellulose membranes were blocked in 5% nonfat milk in 0.05% (v/v) Tween 20 (PBST) (Corning, Manassas, VA). Membranes were then incubated with primary antibodies p-gp130 Y814 (Evseenko lab, AbClonal), p-AKT (cat #4060, Cell Signaling), p-ERK 1/2 (cat #9106, Cell Signaling), p-Y416-SRC (cat #1246F, Novus Biologicals), p-gp130 (cat #1453R, Bioss USA) and p-NFκBp65 (cat #8242, Cell Signaling), and/or YAP1 (cat #4912, Cell Signaling). Histone H3 (cat #9515, Cell Signaling) was used as loading control. After washing in PBS containing 0.05% (v/v) Tween 20 (PBST), membranes were incubated with rabbit (cat #31460, Thermo Scientific) or mouse IgG-HRP (cat #31430, Thermo Scientific) secondary antibodies. After washing, membranes were developed with the Clarity Western ECL Blotting Substrate (Hercules, CA). Quantification of blots was performed using ImageJ 1.44 followed by Prism9 for analysis.

Lizard Tail Healing Model. Captive bred and raised bearded dragon lizards (Pogona vitticeps) were maintained at 25.5° C. with 65% humidity on a 12 hr light/12 hr dark schedule with UVB lamp (Zoo Med) treatment during daylight hours. Care and experimental use of lizards was conducted in accordance with Institutional Animal Care and Use Committee (IACUC). Lizard tails were amputated halfway along lengths with scalpels To systemically deplete macrophage populations, lizards received intraperitoneal (IP) injections of L-α-phoshatidylcholine/cholesterol liposomes containing clodronate (0.125 mg/g) as previously described (Londono et al. J Immunol Regen Med. 2020). Control animals were treated with liposomes containing PBS instead of clodronate. Lizards were treated with R805 drug via daily direct injection (1 pmol drug) into amputated tail stumps. Lizard tail samples were collected 14 days following original tail amputation. Collected tail samples were photographed using an Olympus SZX16 stereo microscope. Images were uploaded in ImageJ (NIH, Bethesda MD) and quantified using the measure command. For labeling of proliferating cell populations with 5-ethynyl-2′-deoxyuridine (EdU) (ThermoFisher Scientific), animals received IP injections of EdU (50 mg/kg) four hours prior to sample collection. Samples to be analyzed via histology were fixed overnight in 4% paraformaldehyde, decalcified for 3 weeks in 10% EDTA (pH 7.4), equilibrated to 30% sucrose, embedded in optimal cutting temperature compound (OCT, Tissue-Tek), crypsectioned at 16 μm thickness, mounted on glass slides, and stained with 4′,6-diamidino-2-phenylindole (DAPI). Samples were stained with Masson's trichrome (American Mastertech) according to the manufacturer's instructions or immunostained for CTSK (Abcam antibody ab19027, used at 1:1000). Images were captured with an Olympus CKX41 microscope outfitted with a Keyence BZ-X800 microscope, and the areas of EdU signal were quantified with ImageJ. Statistical analysis was performed using Prism 7 with one or two-way ANOVA with pairwise Tukey's multiple comparison test for data with multiple groups. A p-value of <0.05 was deemed to be statistically significant. All values and graphs are shown as mean±SD.

RNA sequencing library preparation and sequencing. Total RNA was isolated using QIAGEN RNeasy Mini kit and quantified using Qubit fluorometer (Thermo Fisher Scientific). Quality of the isolated RNA was checked using Agilent Bioanalyzer 2100. Universal Plus mRNA-Seq Library with NuQuant (TECAN) was used to generate stranded RNA-seq libraries. Briefly, poly(A) RNA was selected followed by RNA fragmentation. Double stranded cDNA was generated thereafter using a mixture of random and oilgo(dT) priming. The library was then constructed by end repairing the cDNA to generate blunt ends, ligation of Unique Dual Index (UDI) adaptors, strand selection and PCR amplification. Different adaptors were used for multiplexing samples in one lane. Sequencing was performed on Novaseq SP with paired end 50 base pair reads. Data quality check was done on Illumina SAV. Demultiplexing was performed with Illumina CASAVA 1.8.2.

RNA Sequencing Data Analysis. Raw fastq files were analyzed in Partek flow (version 10.0.21.0801). Reads were aligned to mouse GRCm38 (mm10) genome using Gencode Release M25 reference using STAR aligner (version 2.7.3a) (18). Transcript levels were quantified to the reference using Partek E/M with default parameters. Normalization was done using counts per million (CPM) method. Genes were considered to be differentially expressed based on fold change>2 and p-value<0.05. Functional enrichment analysis for the differentially expressed genes was performed using Ingenuity Pathway analysis (IPA, Qiagen). Pathway schematics were generated using Path designer application of IPA. MA-plots for the differentially expressed genes were generated in R using ggmaplot function of ggpubr (v0.4.0) package.

Single-cell sequencing using 10× Genomics. Single cell samples were prepared using Next GEM Single Cell 5′ v2 (dual index) and Chip Kit (10× Genomics) according to the manufacturer's protocol. Briefly samples were FACS sorted using DAPI to select live cells followed by resuspension in 0.04% BSA-PBS. Nearly 1200 cells/μl were added to each well of the chip with a target cell recovery estimate of 10000 cells. Thereafter Gel bead-in Emulsions (GEMs) were generated using GemCode Single-Cell Instrument. GEMs were reverse transcribed, droplets were broken and single stranded cDNA was isolated. cDNAs were cleaned up with DynaBeads and amplified. Finally, cDNAs were ligated with adapters, post-ligation products were amplified, cleaned-up with SPRIselect and purified libraries were sequenced on Novaseq.

10× Sequencing data analysis. Raw sequencing reads were processed using Partek Flow Analysis Software (build version 10.0.21.0210). Briefly, raw reads were checked for their quality, trimmed and reads with an average base quality score per position>30 were considered for alignment. Trimmed reads were aligned to the dog genome version canFam3-Ensembl_v102 using STAR-2.6.1d with default parameters. Reads with alignment percentage >75% were deduplicated based on their unique molecular identifiers (UMIs). Reads mapping to the same chromosomal location with duplicate UMIs were removed. Thereafter ‘Knee’ plot was constructed using the cumulative fraction of reads/UMIs for all barcodes. Barcodes below the cut-off defined by the location of the knee were assigned as true cell barcodes and quantified. Further noise filtration was done by removing cells having >5% mitochondrial counts and total read counts >24,000. Genes not expressed in any cell were also removed as an additional clean-up step. Cleaned-up reads were normalized using counts per million (CPM) method followed by log-transformation generating count matrices for each sample. Samples were batch-corrected on the basis of expressed genes and mitochondrial reads percent. Count matrices were used to visualize and explore the samples in further details by generating tSNE plots, a non-linear dimensional reduction technique. This algorithm learns the underlying manifold of the data and places similar cells together in a low-dimensional space. Gene ontology enrichment analysis for the differentially expressed genes was performed using DAVID Gene Functional Classification Tool ([http://]david.abcc.ncifcrf.gov; version 6.8). Dot plots were generated in R (v4.0.3) using ggplot2(v3.3.3).

Differential expression analysis. Reads were aligned to mouse GRCm38 (mm10) genome using Gencode Release M25 reference using STAR aligner. Normalization was done using counts per million (CPM) method. Transcript levels were quantified to the reference using Partek E/M with default parameters. Genes were considered to be differentially expressed based on fold change>2 and p-value<0.05. Functional enrichment analysis for the differentially expressed genes was performed using Ingenuity Pathway analysis (IPA, Qiagen). Pathway schematics were generated using Path designer application of IPA. MA-plots for the differentially expressed genes were generated in R using ggmaplot function of ggpubr (v0.4.0) package.

Data availability. All RNA-seq data for F814 mouse are deposited in GEO under the SuperSeries accession number GSE168279. Single sequencing data of canine synovium are deposited in GEO under the accession number GSE168395.

Quantitative Real-Time PCR. Power SYBR Green (Applied Biosystems) RT-PCR amplification and detection was performed using an Applied Biosystems Step One Plus Real-Time PCR machine. The comparative Ct method for relative quantification (2-ΔΔCt) was used to quantitate gene expression. TBP (TATA-box binding protein) was used for gene normalization and expressed relative to a calibrator (sample in each set with lowest expression). Q-PCR for was conducted using the following primers: human: TBP forward, 5′ GATGGACGTTCGGTTTAGG 3′ (SEQ ID NO:28); TBP reverse, 5′ AGCAGCACAGTACGAGCAA 3′ (SEQ ID NO:29), MMP13 forward, 5′ ACTGAGAGGCTCCGAGAAATG 3′(SEQ ID NO:30); MMP13 reverse, 5′ GAACCCCGCATCTTGGCTT 3′ (SEQ ID NO:31), ADAMTS5 forward 5′ GAACATCGACCAACTCTACTCCG 3′ (SEQ ID NO:32); ADAMTS5 reverse, 5′ CAATGCCCACCGAACCATCT 3′ (SEQ ID NO:33), ADAMTS4 forward 5′ GCAACGTCAAGGCTCCTCTT 3′ (SEQ ID NO:34); ADAMTS4 reverse, 5′ CTCCACAAATCTACTCAGTGAAGCA 3′ (SEQ ID NO:35), Collagen II forward, 5′ CCTGGCAAAGATGGTGAGACAG 3′ (SEQ ID NO:36); Collagen II reverse 5′ CCTGGTTTTCCACCTTCACCTG 3′ (SEQ ID NO:37); Aggrecan forward, 5′ AGGCAGCGTGATCCTTACC 3′ (SEQ ID NO:38); Aggrecan reverse, 5′ GGCCTCTCCAGTCTCATTCTC 3′ (SEQ ID NO:39). Cells were treated with 10 ng/mL OSM and/or with 5 μM SRC (SU6656) inhibitor, 10 ng/mL OSM and/or 50 ng/mL WT/F814 plasmids; 10 ng/mL OSM and/or 100 or 300 ug/mL of peptide QQpYF (Evseenko lab, Thermo Scientific); 10 uM R805 and/or 10 ng/mL OSM.

Cloning and transfection of Ba/F3 and ATDC5 cells. A plasmid encoding full-length gp130 (WTgp130; InVivoGen) was used to create the 5 mutants. The parental vector was digested with EcorI and NheI; 5 different DNA fragments encoding point mutations for tyrosine to phenylalanine (residue Y814), lysine to arginine (residue K816), and serine to alanine (residues S820, S824, S825) within 5′ GATGGTATTTTGCCCAGGAGTCAGCATGAATCCAGT 3′ (SEQ ID NO:40) d812-827a were purchased from Thermo Scientific and ligated into the backbone. All independent transfectants were sequenced to verify the point mutation substitution and transfected into Ba/F3 cells or ATDC5 cells. 48 hours after transfection of either WT gp130 or 5 mutants, cells were treated with or without 10 ng IL-6, OSM and/or 5 uM of SRC inhibitor SU6656 and then harvested for Western blot.

Immunohistochemistry. Histological staining for was performed on wild type (WT) mice, F814 mice and all mice that underwent destabilization of the medial meniscus (DMM). Six weeks after surgery, mice were sacrificed and joints were fixed in 10% formalin, decalcified in 10% EDTA and embedded in paraffin. For rat model, 6 weeks after partial medial meniscectomy (PMM), rats were sacrificed and joints were fixed in 10% formalin, decalcified in 10% EDTA and embedded in paraffin. A total of three 8 μm thick sections were made at a 200 μm interval by coronal sectioning. For canine model, 4 and 16 weeks after medial meniscal release (MMR), dogs were sacrificed and joints were fixed in 10% formalin, decalcified in 10% EDTA and embedded in paraffin. No animals were excluded from analysis. A microtome (Leica) was used to cut 5-μm sections for joints. H&E staining was performed to assess morphology. Safranin O/Fast Green staining, immunohistochemistry for proteolytic enzymes and Osteoarthritis Research Society International (OARSI) scoring was performed as described (41, 42). For OARSI scoring, observers performing the analysis were blinded as whether the slides were from treated or control animals. The stained samples were visually observed using LSCM (Zeiss LSM710, Carl Zeiss, Germany).

Neoepitope analysis. WT and F814 mouse articular cartilage explants were treated with or without OSM (10 ng/mL). Pig articular cartilage explants were stimulated with or without OSM (10 ng/mL) and treated with or without peptide QQpYF (100 μg/mL or 300 μg/mL). For R805 treatment, pig articular cartilage explants were stimulated with or without OSM (10 ng/mL) and treated with or without R805 (0.01, 0.1, 1 or 10 μM). The explants were then digested for 2 hr at 37° C. with 0.01 units chondroitinase ABC (Sigma Aldrich, St. Louis, MO). Samples were then dialyzed with ultrapure water for 24 hr at 4° C., freeze dried, dissolved in RIPA Lysis and Extraction Buffer (Pierce, IL) containing protease inhibitors (Pierce) followed by sonication. Proteins were separated and analyzed by Western blot using primary antibodies against aggrecan (cat #NB100-74350, Novus Biologicals,) and collagen II neoepitopes (cat #50-1035, Ibex). Wet weight of explants was used as loading control.

Peptide QQpYF synthesis. Amino acid sequence of human gp130 was used and peptides were synthesized surrounding tyrosine 814. The peptide sequences synthesized in vitro in a cyclic manner were as follows: RQQYFKQNCSQHESS (SEQ ID NO:41), DGILPRQQYFKQNCS (SEQ ID NO:42), YFKQNCSQHESSPDIS (SEQ ID NO:43), GDGILPRQQYFKQN (SEQ ID NO:44), QYFKQNCSQHESSP (SEQ ID NO:45), VDGGDGILPRQQYFK (SEQ ID NO:46), DGGDGILPRQQYFK (SEQ ID NO:47), QYFKQNCSQHESSPD (SEQ ID NO:48), DGGDGILPRQQYFKQN (SEQ ID NO:49), GDGILPRQQYFKQNC (SEQ ID NO:50), GGDGILPRQQYFKQN (SEQ ID NO:51), GILPRQQYFKQNCSQ (SEQ ID NO:52), PRQQYFKQNCSQHE (SEQ ID NO:53), PRQQYFKQNCSQHES (SEQ ID NO:54), QQYFKQNCSQHE (SEQ ID NO:55), QQYFK (SEQ ID NO:56), QQYF, QYFK, YFK, QQY, and QYF. (4 mg, purity >=50%) with >70% conjugation rate of peptide to protein carriers. All tyrosines (Y) were modified to phosphorylated tyrosines. The peptides were synthesized and provided by Thermo Scientific (Rockford, IL).

Molecular docking and Molecular Dynamics Simulations. The molecular docking of Gln-Gln-phosphoTyr-Phe (QQ[pY]F) to c-Src kinase was performed in two steps. The coordinates for the c-Src Kinase was obtained from the protein data bank (PDBID 2SRC). In the first step, protein-peptide docking server was used to identify the preferred binding site in the c-Src kinase for the QQYF peptide. For the blind c-Src Kinase-QQ[pY]F docking, HPEPDOCK server ([http://]huanglab.phys.hust.edu.cn/hpepdock/) was used. HPEPDOCK server uses a hierarchical flexible-peptide docking protocol, which includes conformational sampling of the peptide and peptide docking. In the second step, refinement of the protein-peptide docking was done using the GOLD, version 5.8.1 using the best ranked site for QQ[pY]F binding to SRC kinase that was identified by the HPEPDOCK server as a starting model. All the residues within 18 Å of centroid around the initial identified site were defined as part of the peptide binding site. The GOLD refinement protocol includes 300 genetic algorithm run, 100 000 iterations were employed in which early termination option was disabled. The best docked pose was then chosen based on the GOLDSCORE and all poses were retained. Molecular dynamics (MD) simulations was carried out to determine the binding free energy of QQ[pY]F peptide binding to cSrc Kinase. AMBER18 package was used to perform the MD simulation. Amber-compatible parameters for post-translational modified amino acids were taken from Forcefiled_PTM. After adding hydrogens, the cSrc Kinase-QQ[pY]F complex was solvated in a truncated octahedral TIP3P box of 12 Å, and the system was neutralized with sodium ions. Periodic boundary conditions, Particle Mesh Ewald summation and SHAKE-enabled 2-femto seconds time steps were used. Langevin dynamics temperature control was employed with a collision rate equal to 1.0 ps⁻¹. A cutoff of 13 Å was used for nonbonding interactions. Initial configurations were subjected to a 1000-step minimization with the harmonic constraints of 10 kcal·mol⁻¹A°⁻² on the protein heavy atoms. The system was gradually heated from 0 to 300 K over a period of 50 ps with harmonic constraints. The simulation at 300 K was then continued for 50 ps during which the harmonic constraints were gradually lifted. The system was then equilibrated for a period of 500 ps before the 100 ns production run. The MD simulation was carried out in the NPT ensemble. Equilibration and production run were carried out using the Sander and PMEMD modules (optimized for CUDA) of AMBER 18.0 (ff14SB). All analyses were performed using the cpptraj module of AmberTools 18. From the 100 ns simulation 10000 structures were taken at an interval 10 ps for the free energy calculations. The MMPBSA module in AMBER was used to compute the binding free energy for QQ[yP]F binding to cSrc kinase.

Generation of F814 CRISPR/Cas9 mouse. Selection and synthesis of Cas9 mRNA and sgRNA (5′ AAAATGTGAAATCTCTGGACAGG-3′(SEQ ID NO:57)) to gp130 target region was provided by PNA Bio (Thousand Oaks, CA) and targeting efficiency of the sgRNAs used for the knock-in experiment was evaluated by surveyor nuclease assay to detect the sgRNA with highest DNA cleavage efficiency. Microinjections by USC Transgenic Core were performed at one-cell stage embryo using C57BL/6 mouse strain. Mice genotyping was performed by GeneWiz (La Jolla, CA).

Destabilization of the Medial Meniscus (DM4) mouse model of osteoarthritis. Six 3-month-old wild type (Charles River, USA) and F814 mice (University of Southern California, USA) were anesthetized and medial para-patellar arthrotomy was carried out under a dissection microscope to expose the meniscus via knee flexion; once located, the meniscus ligament was cut, but not removed, to destabilize the joint. Only males were used in this study; females are resistant to this injury type of injury. Six weeks after the initial surgery, mice were sacrificed and the joints were transferred to 10% formalin for 48 hours followed by a decalcification process and embedding in paraffin for histological assessment.

Wound-induced hair neogenesis assay. The WHIN assay was previously described. Briefly, a 1.5×1.5 cm square full thickness wound was excised on the posterior dorsum of 6-week-oldf mice, and observed for hair neogenesis and wound histology. Mice were anesthetized using isofluorane, full thickness skin was excised, and analgesic Buprenorphine SR (0.5 mg/kg) was given by intraperitoneal injection (IP) at the beginning of the procedure. Additional DietGel Boost (ClearH2O) was placed on the bottom of the cage during the first week post-operation. For R805-treatment, R805 (10 μM or 20 μM) was applied topically to the wound of WT mice.

Alkaline phosphatase (ALP) stain. To detect newly forming dermal papillae, alkaline phosphatase staining was performed as previously reported. Briefly, full thickness wounds were excised and epidermis separated from the dermis using 20 mM EDTA. The dermis was fixed in acetone overnight at 4° C., and washed in PBS several times. The dermis was pre-incubated in ALP buffer (0.1 M Tris-HCl, 0.1 M NaCl, 5 mM MgCl₂ and 0.1% Tween-20) for 30 min, incubated with BCIP/NBT Color Development Substrate (Promega, Madison, WI, USA) in ALP buffer at 37° C. until color development. The reaction was stopped by washing with pH 8.0 Tris-EDTA and the tissue stored in PBS with sodium azide. 5 wounds were calculated for each strain of mice.

Hair fiber length quantification. To quantify regenerated hair fiber length, hair fibers from respective wounds were plucked, aligned and photographed next to a ruler under the dissecting microscope. The length of the hair fibers was measured and analyzed using ImageJ. 3-4 hairs were randomly plucked from each wound and analyzed.

Synthesis of phospho-gp130 Y814 antibody. Phospho-gp130-Y814 antibody was synthesized and provided by AbClonal (Woburn, MA). Synthesis of antigen modified (phosphorylated) peptide: PRQP(pY)FKQNC (SEQ ID NO:58) (19 mg, purity>=85%), non-modified peptide: PRQPYFKQNC (SEQ ID NO:58) (14 mg, purity>=85%), and conjugation of modified peptide to KLH was performed followed by immunization of 3 New Zealand rabbits and sera collection. Antibody purification was conducted by antigen affinity chromatography followed QC via dot-blot test against the modified and non-modified polypeptides.

Receptor competition assay. Pig articular chondrocytes were transfected with gp130-Flag 72 hours before incubation with 0.3, 1, 3, 10 or 30 μM R805 in the presence of 10 ng/mL OSM. After 24 hours of cytokine treatment, protein was extracted and immunoprecipitated with gp130-Flag (cat #125623, Thermo Scientific). Western blots were then performed for detection of OSMR (cat #ab85575, Abcam) and antibodies and normalized to gp130-Flag (cat #125623, Thermo Scientific).

Rat model of osteoarthritis. All experiments were conducted ethically. 24 male Sprague-Dawley (10 weeks old) were purchased through Charles River, USA. Animals were pair housed in standard cages in a temperature and humidity regulated room on a 12h dark/light cycle. 8 Rats received medial meniscal tear (MMT) surgery and weekly injections of the vehicle (50 microliter of Saline+DMSO 1:1000), another 8 rats received MMT surgery and 50 uL at 10 μM of R805 and 3 rats were used as sham operated control. All surgeries were performed on the right hind paw. Rats received weekly injections with either the drug or the vehicle and an empty needle in the case of the shams. Rats were anesthetized using 5% Isoflurane (VetOne inc., Boise, ID, USA) in 100% oxygen and maintained with 2% Isoflurane throughout the surgery. The joint was held in a 90-degree angle to open the joint space. Soft tissue was bluntly dissected to expose the meniscus and carefully cut to excise ˜50%. Rats were sacrificed 6 weeks' post-surgery, joints were harvested and kept in a PBS drenched cloth in a humid chamber at 4 degrees Celsius for a maximum of 8 hours. Indentation was performed on fresh joints, and once finished, the joints were transferred to 10% formalin for 48 hours followed by a decalcification process and embedding in paraffin for histological assessment.

Indentation and thickness mapping. Mechanical properties were mapped ex vivo using a 17N multi-axial load cell and a 0.5 mm spherical indenter. The Mach-1 v500css, a novel developed device by Biomomentum Inc., Laval, Canada, was utilized for cartilage indentation and thickness mapping on rat articular cartilage. A camera system provided by Biomomentum was used to superimpose a position grid that was used as a template to predefine 40 positions per tibia, 40 per femur and 20 per patella. Indentation was performed at a speed of 50 μm/s using an amplitude of 50 μm, scanning grid was set on 100 μm. When indentation was finished, the spherical indenter was replaced with a 26G ½″ intradermal bevel needle to determine the thickness of the predefined positions to calculate the instantaneous modulus using the model developed by Hayes. Instantaneous modulus (IM) and thickness data is presented as mean+/−SD. IM data shown represents the instantaneous modulus at 20% strain to mimic its compression in vivo.

Pharmacokinetic assessment of R805. Pharmacokinetics were conducted in a canine model for over 90 days. Purpose-bred beagles with intact stifles were injected intra-articularly with 500 μL of physiological saline containing 0.25% carboxymethyl cellulose (CMC) and either 1 μg or 100 ng of CX-011 (n=4 each). Synovial fluid and plasma were collected at d0, d3, d7, d14, d21, d31, d60 and d90 and analyzed by LC-MS/MS. Doses of 10 μg, 1 μg and 0.1 μg of R805 were utilized for the main experiment.

Canine osteoarthritis model. All procedures were approved by an institutional animal care review board using national guidelines governing research animal welfare. Twenty-four purpose bred Foxhound cross dogs (12 females, 12 males), 10 months of age (Marshall BioResources (North Rose, NY, USA)) were chosen based on ability to walk on a leash and social interaction with handlers. Littermate information was obtained to ensure littermates were placed in separate groups to control for genetic similarities. Dogs were allocated to groups to approximate similar body weight and gender distributions. Additional daily socialization and outdoor exercise was done with trained handlers, and weekly training on an obstacle course were done for four weeks. Two weeks pre-operatively all dogs underwent baseline assessments including: lameness scoring (0 to 5) where 0 is normal and 5 is non-weight bearing, Colorado Acute Pain Scale scoring (0-4), and gait analysis using a Tekscan Walkway7 (Tekscan Inc., Boston, MA, USA). Five valid gait trials were completed for each dog at each time point. A trial was considered valid when three complete gait cycles were recorded and velocity was within 1.6-1.9±0.5 m/s (Brown et al., 2013). All data was collected using Tekscan Walkway Research Software (ver. 7.66-05) and forces were normalized to each dog's body weight (kg). Maximum force (kg) and maximum peak pressure (kPa) for each limb as well as maximum force ratios between front/hind, left front/right front and left hind/right hind were selected for analysis. At week 0 CT imaging of both knee joints was done under general anesthesia which was followed by unilateral arthroscopic transection of the caudal medial meniscotibial ligament. This medial meniscal release procedure (MMR) resulted in loss of meniscal loading sharing in order to induce OA in one knee. Dogs were allocated into four experimental groups (n=6). Three groups received CX-011 by intra-articular injection into the operated limb and the fourth control group (n=6) received an equal volume of vehicle. Intra-articular treatments were done four weeks postoperatively and repeated a second time at week 11. The three experimental groups given the test article received doses of concentration of 1 μg, 0.1 μg or 0.01 μg in a 1.0 mL volume. Knee joint CT imaging was repeated at 4 weeks and again at 16 weeks to assess OA progression. At 18 weeks the dogs were euthanized using pentobarbitol and the knee joints were harvested. MicroCT imaging at 45p resolution using a GE eXplore Locus platform (GE Healthcare/EVS Corp., London, ON, Canada) allowed 3D reconstructions to be created and adaptive regions of interest 3 mm deep were made in the central non-meniscus covered portion of the tibial plateau subchondral bone plate. Bone morphometry parameters including tissue mineral density and trabecular bone volume were calculated using Microview^{T}M analytical software (Parallax Innovations Ltd., London, ON, Canada). Following this the knee joints were dissected for macrophotography and biomechanical indentation testing. Thickness and instantaneous modulus were calculated for X points on the medial tibia and Y points on the medial femoral condyle using a 1 mm spherical indentor, a 0.2 mm/s rate with a 5 second relaxation time. The spherical indenter was replaced with a 26 G ⅜″ precision needle and an automated needle penetration test (0.5 mm/s up to a predefined load to reach subchondral bone) was performed at the same pre-defined mapping points to determine cartilage thickness. Heat scale color scale maps of modulus and thickness were created. Collection of synovial tissues for histology included synovial membrane tissue sections that were stained with H&E and osteochondral sections of the medial femoral condyle and tibial plateau that were stained with both H&E and safranin-O. All sections were reviewed by one investigator blind to the treatment allocations using the canine OARSI osteoarthritis score system.

A gp130 modality is responsible for mediating cytokine-induced pro-inflammatory and pro-fibrotic signaling cascade. The molecular inflammatory processes that are activated to promote regeneration in an attempt to reestablish homeostasis after an acute injury can, when overstimulated, progressively drive degeneration of tissue and fibrosis, which is a pathogenic process where connective tissue replaces normal parenchyvmal tissue forming a permanent scar. Inflammation mediated by IL-6 family cytokines has been identified as a driver of chronic inflammatory process and fibrosis. Engagement of gp130 by IL-6 cytokines leads to activation of various downstream signaling modules including the JAK/STAT3, MAPK/ERK, PI3K/AKT and less renowned SRC signaling but the role of each of these modules and their interactions are context specific and not fully understood.

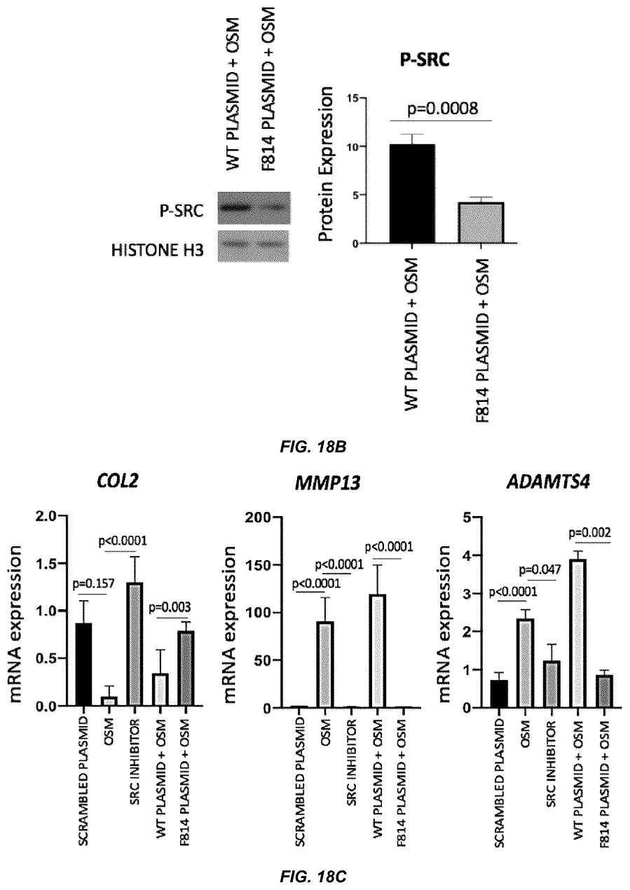

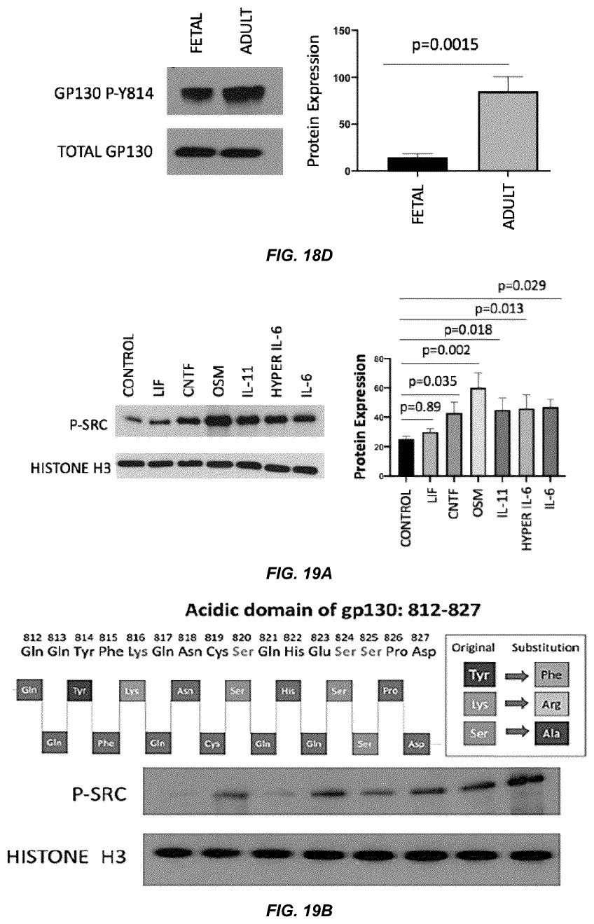

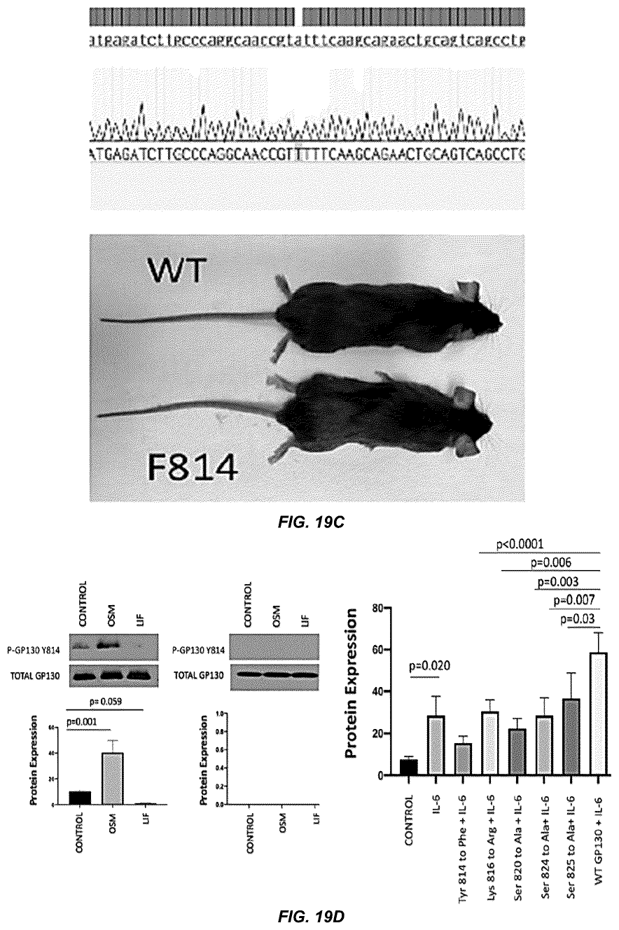

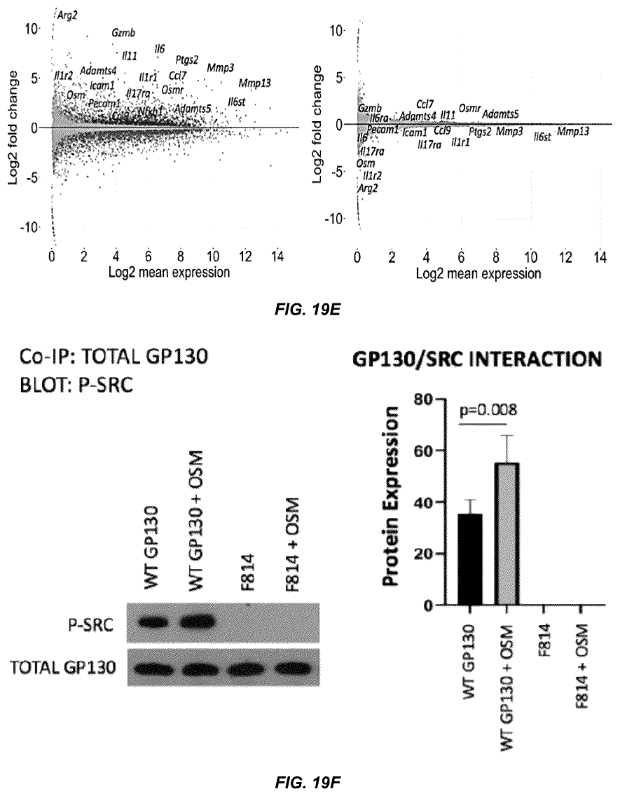

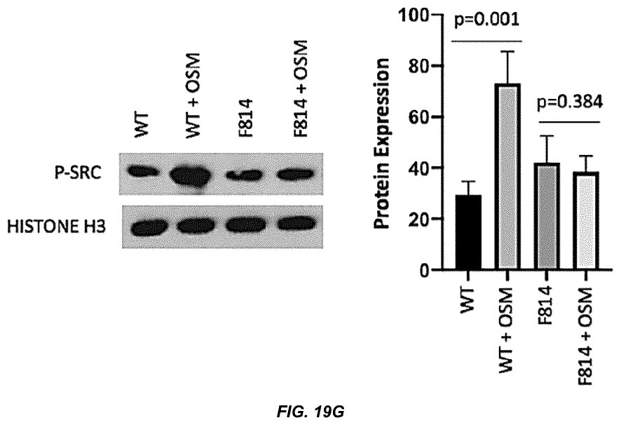

Experiments were performed to assess which potential residue plays the most predominant role in tissue degradation downstream of gp130, more specifically, degradation of extracellular matrix. To test this, pig articular chondrocytes were stimulated with OSM, which was the most pro-inflammatory IL-6 family member, followed by treatment with numerous pharmacological inhibitors to block gp130-dependent signaling. The results demonstrated that relative to other pathways, inhibition of SRC kinase had the most significant effect of abolishing OSM-induced transcription of matrix-degrading genes ADAMTS4 and MMP13 (FIG. 18A). This was of interest because prior studies reported SRC to be highly expressed in the synovium of patients with rheumatoid arthritis and constitutively activated in a rat model of collagen-induced arthritis. To determine which cytokine has the most substantial effect on SRC upregulation, pig articular chondrocytes were stimulated with various IL-6 cytokines and relative to all the cytokines tested, OSM displayed the strongest upregulation of phosphorylated (active) SRC (pSRC) (FIG. 19A). However, the specific mechanism of how gp130 regulates SRC activation was obscure although it is well-recognized that IL-6 ligands induce specific post-translational modifications in gp130 residues to activate signaling.

Based on previously published data identifying acidic domain 812-827 of gp130 as the primary region of SRC activation, the disclosure identifies specific residue responsible for SRC signaling. In order to determine which specific residue was responsible for this activation within residues 812-827, various amino acids were modified within this domain through sequence substitutions using plasmids in vitro. Using Ba/F3 cell line, which is completely deficient of gp130, the cells were transfected with plasmids containing wild type (WT) gp130 or gp130 sequence substitutions and stimulated with IL-6 to induce gp130 signaling. The results delineated that substitution of tyrosine (Y) to phenylalanine (F) at residue Y814 (F814) significantly reduced IL-6-stimulated pSRC (phosphorylated) expression relative to the control WT gp130 plasmid suggesting that Y814 might be responsible for pSRC regulation (FIG. 19B). Experiments were subsequently performed to verify that mutation in Y814 can prevent endogenous recruitment of SRC to gp130 blocking activation of matrix degrading enzymes. To demonstrate this, an ATDC5 cell line was used, which is a chondrogenic cell line where gp130 expression is minimal. Transfection of ATDC5 with F814 plasmid stimulated with OSM markedly reduced pSRC stimulation relative to the control WT gp130 plasmid (FIG. 18B), and in the same cells, there was a decreased expression of MMP13 and ADAMTS4, and an increased biosynthesis as confirmed by an increase in collagen type II (COL2) transcription (FIG. 18C). As such, tyrosine to phenylalanine modification of Y814 confirmed the necessity of this residue for SRC activation in chondrocytes suggesting that Y814 may play a role in cytokine-induced pro-inflammatory and pro-fibrotic signaling cascades downstream of gp130.

A polyclonal antibody was developed against phosphorylated (active) gp130 Y814 (pY814). Since Y814 was shown as potentially inducing biosynthesis, experiments were performed to see if there was a difference in expression of Y814 in a developing anabolic fetal joint verses adult joint. As expected, pY814 activation was significantly lower in fetal chondrocytes compared to adult (FIG. 18D) suggesting that Y814 does not play a major role in rapidly proliferating and highly anabolic primary cartilage cells.

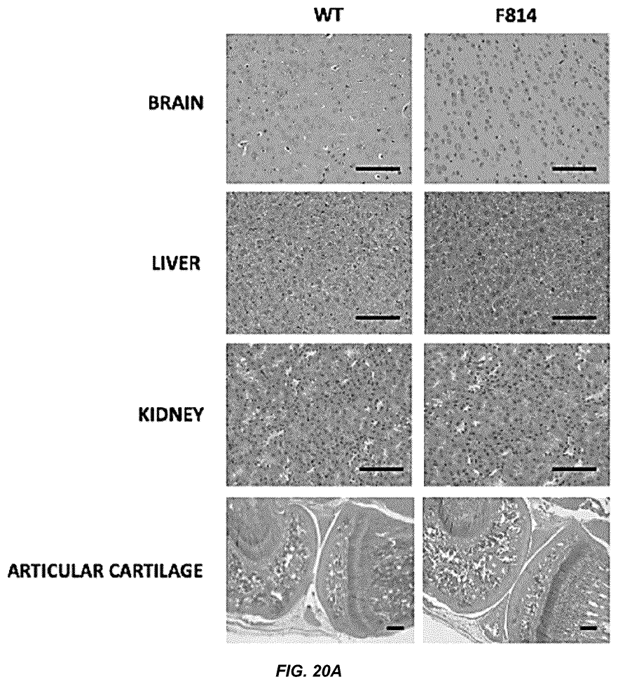

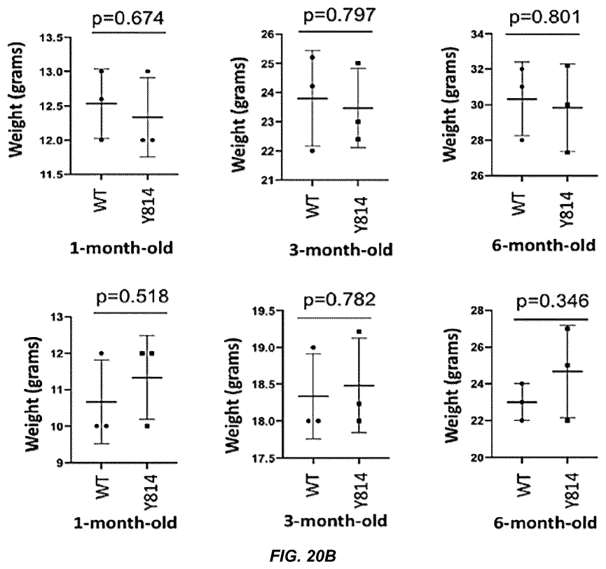

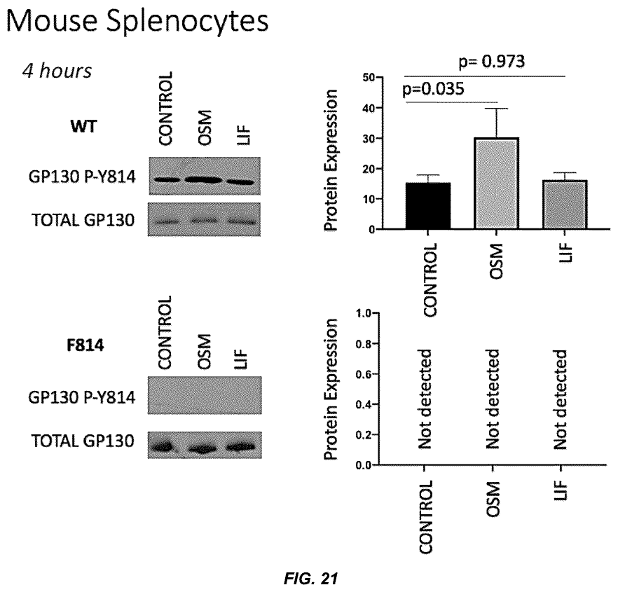

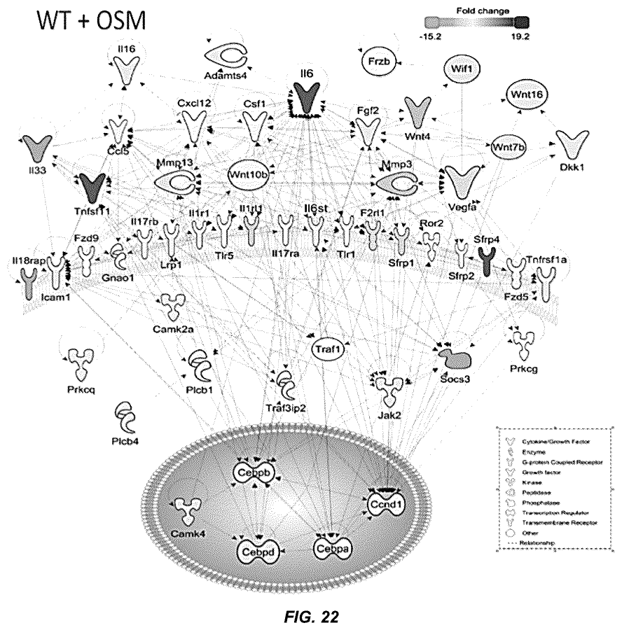

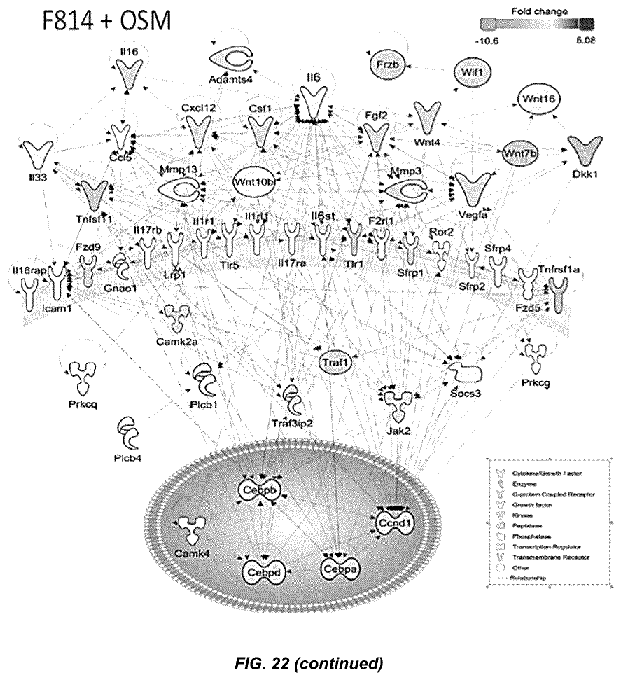

In order to expose the function of this residue in vivo, a CRISPR/Cas9 homozygous murine model was developed with a genetically modified gp130 Y814 (F814). The mouse was validated by functional tests and genetic sequencing. The mutant mouse is viable, fertile, and exhibits no significant morphological differences in the musculoskeletal tissues or other organs relative to the WT (FIGS. 19C and 20). To validate the deletion of Y814 in the mutant mice, F814 mouse splenocytes were used; no Y814 was detected endogenously or in response to OSM treatment though marked activation of this residue was seen in WT cells (FIGS. 19D and 21). Experiments were then performed to identify the potential differences in reactivity to IL-6 cytokines between the WT and F814 mouse. WT and F814 mouse periarticular fibroblast cells were treated with or without OSM and subjected to RNA sequencing (RNA seq). Ordinarily, OSM upregulates a variety of pro-inflammatory and pro-fibrotic genes. Strikingly, RNA seq results demonstrated that deletion of Y814 dramatically reduces expression of these genes, including major proteases (e.g., Mmps 3, 13; Adamts 4,5; Gzmb), cytokines and their receptors (e.g., Il-11, Il-6, Osm, Il-1r1&2, Osmr, Il-17ra), and major pro-inflammatory regulators (e.g., NF-kb1, Ptgs2/Cox2) in response to OSM. Ingenuity Pathway Analysis (IPA) suggested the Role of Macrophages, Fibroblasts and Endothelial Cells in Rheumatoid Arthritis (−log₁₀p-value=7.3 or p-value=5.01187E-08) (FIG. 22) and other pro-inflammatory and fibrosis-related pathways being the most affected by this mutation. MA plots for differential gene expression analysis confirmed that upon treatment with OSM, WT mouse fibroblasts upregulate a variety of pro-inflammatory and pro-fibrotic genes while F814 cells do not show much change in downstream gene expression (FIG. 19E) alluding to the fact that Y814 is a major stress sensor. Sequentially, mutant-derived splenocytes revealed no endogenous interaction of SRC with gp130 as shown by co-immunoprecipitation (FIG. 19F) and no activation of pSRC after stimulation with OSM stimulation was detected (FIG. 19G).

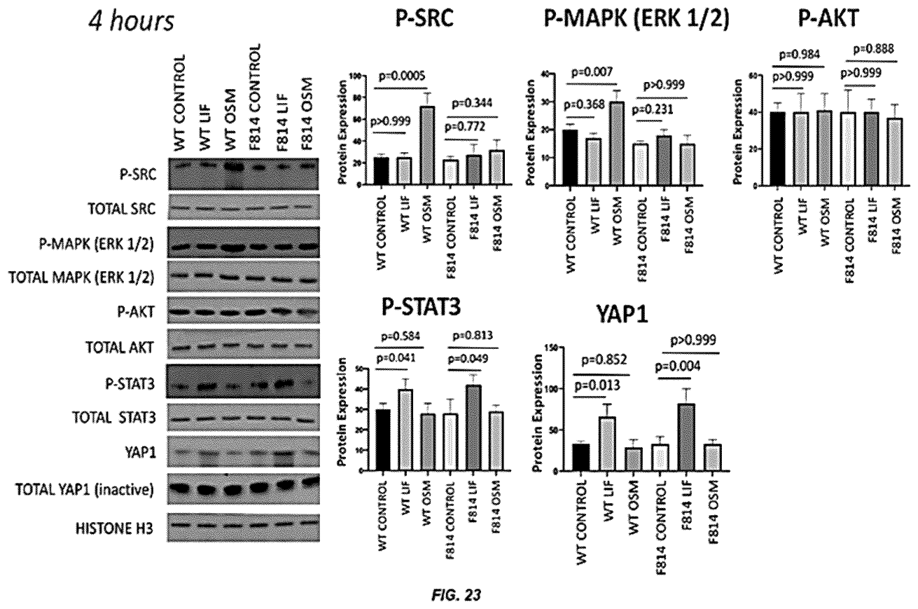

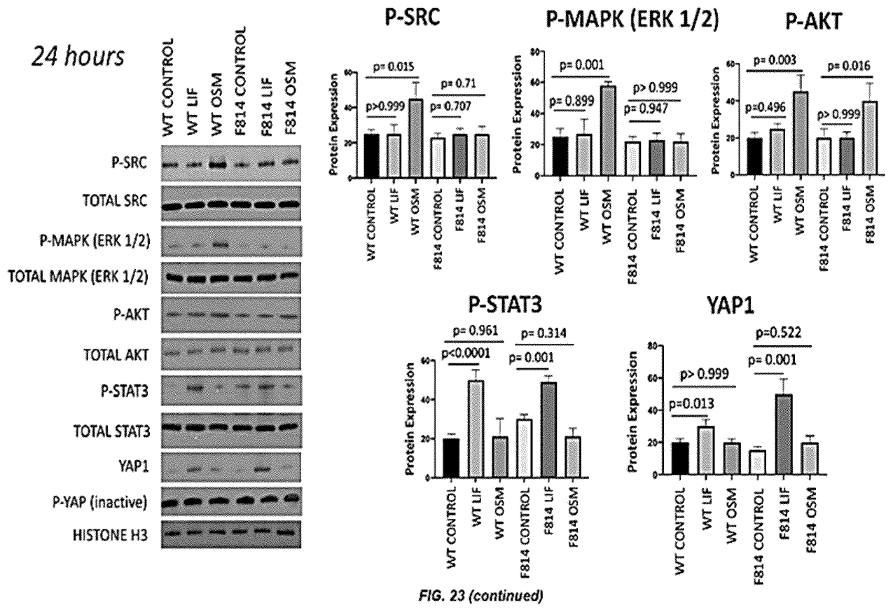

STAT3 and YAP signaling pathways downstream of gp130 play a major role in regeneration, development, and diseases such as osteoarthritis. Stimulation of WT and F814 mouse primary periarticular fibroblasts with LIF suggested that gp130 Y814 residue is minimally responsible for activation of STAT3 or YAP signaling as the mutant cells were not resistant to LIF-stimulated phosphorylated (active) STAT3 (pSTAT3) or YAP1 mirroring WT cells (FIG. 23). STAT3 and YAP1 levels were evaluated in response to LIF downstream of gp130 since LIF has been shown to primarily signal through STAT3 in chondrocytes. It is also noteworthy to mention that in WT mouse splenocytes, treatment with LIF did not upregulate gp130 pY814 further confirming that LIF is not involved in Y814 signaling (FIGS. 19D and 21). Although Y814 may be one of the four residues responsible for STAT3 recruitment, it may not be the primary docking site, which would explain why this lack of STAT3 regulation in the F814 cells was observed. Simultaneously, genetic ablation of Y814 has completely blunted OSM-mediated activation of SRC and MAPK/ERK signaling further supporting its role exclusively in pro-inflammatory and pro-fibrotic signaling (FIG. 23).

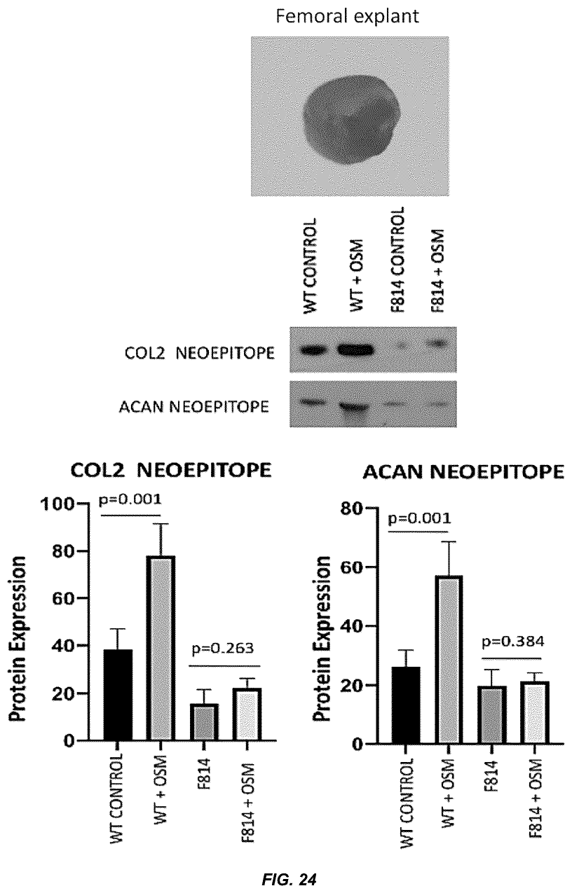

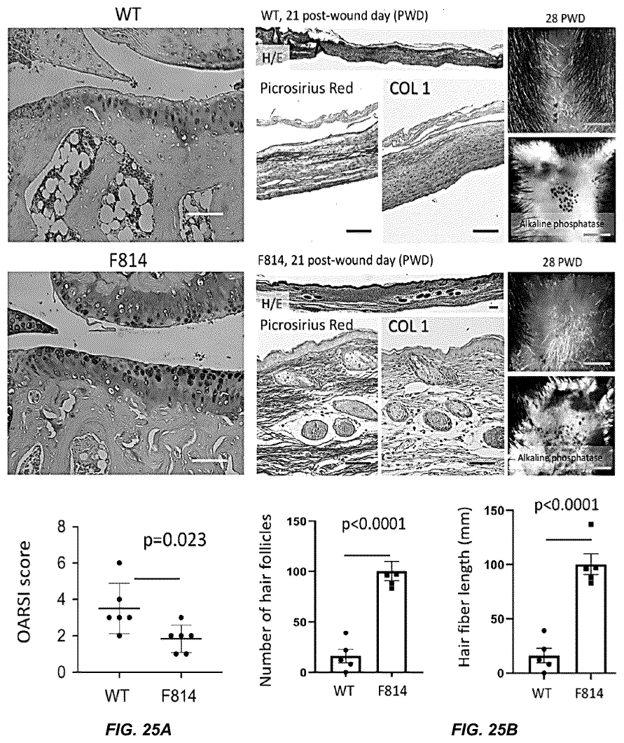

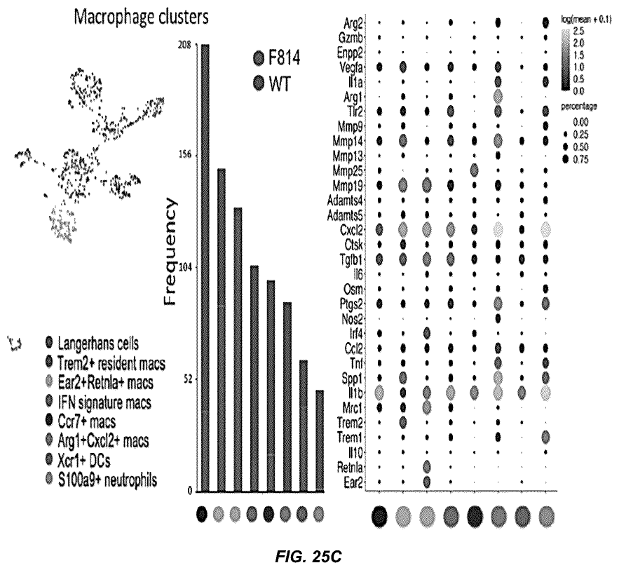

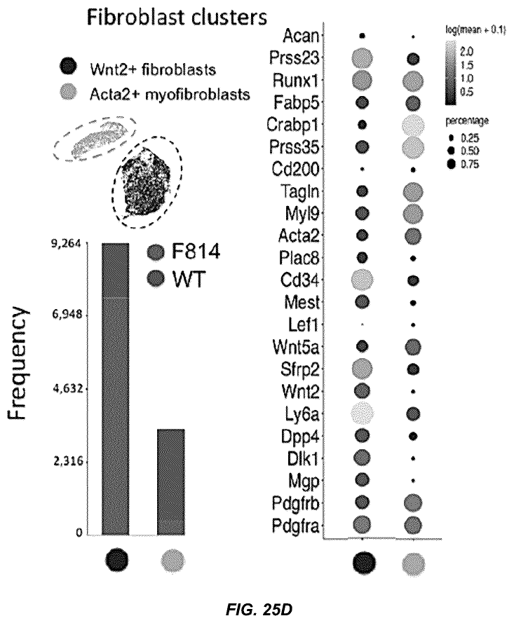

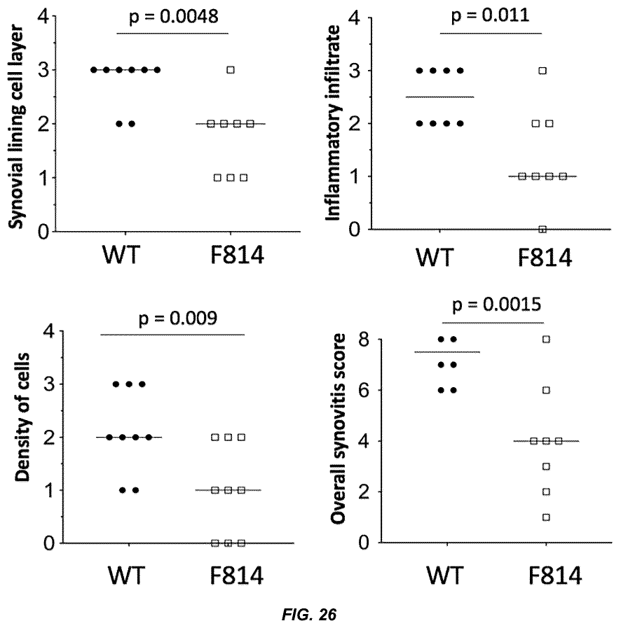

To assess the direct effect of the Y814 mutation on matrix loss, a neoepitope assay was performed using isolated femoral head explants cultured in the presence or absence of OSM. The results confirmed a significant decrease in cartilage degeneration in femoral head of Y814 mice relative to the WT as shown by the low aggrecanase and collagenase activity in the mutant (FIG. 24). To address whether Y814 mutation can ameliorate cartilage degeneration in joint disease, a destabilization of the medial meniscus murine model was used. This experiment clearly showed that F814 mice are resistant to surgically-induced OA as reflected by reduced loss of proteoglycans, reduced synovitis and synovial fibrosis (FIGS. 25A and 26).

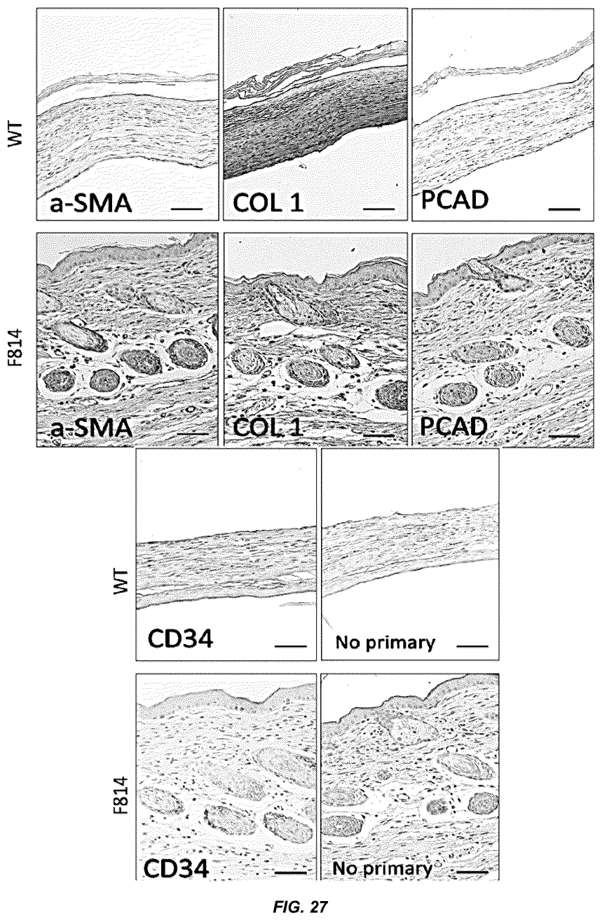

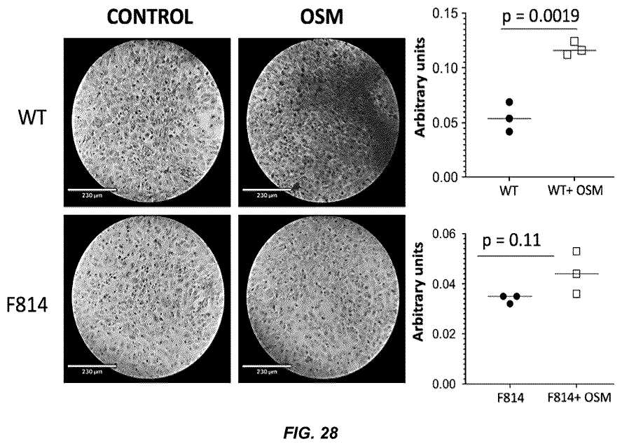

As previously mentioned, pro-inflammatory reactions resulting from tissue damage, if modulated, can contribute to successful regeneration of tissue. Since ablation of Y814 prevents aberrant inflammatory signaling initiated by pro-inflammatory cytokines while bypassing the pro-regenerative signaling in vitro, experiments were performed to determine whether F814 mouse has superior regenerative potential in vivo. Unfortunately, OA animal models are not well-suited to study regeneration; thus, a canonical skin excisional wound model was used where the regenerative ability of the WT and F814 mouse skin could be assessed and compared using the wound-induced hair neogenesis (WIHN) assay as evidence of hair follicle neogenesis and dermal regeneration. The histological observations after the procedure show, at the same magnification, that the post-wound day (PWD) 21 WT wounds are thinner and very few hair placodes are observed (FIG. 25B). Early hair placode marker P-cadherin and hair follicle bulge cells surface marker CD34 were stained negative in the wound (FIG. 27). On the contrary, the WT wound expressed high levels of notorious pro-fibrotic markers, α-SMA and COL1, and positively stained for Picrosirius red in the dermis showing high collagen deposition in addition to being contracted indicating fibrosis (FIG. 25B). In the PWD 21 F814 mouse sections, the wound is thicker and many prominent newly formed hair follicles were observed; α-SMA, CD34 and P-cadherin were expressed mostly within the hair placodes and follicles (FIGS. 25B and 27). In addition, the intensity of Picrosirius red and COL1 staining in the wound dermis was lower in F814 mouse relative to the WT mouse, and the wound was not as contracted signifying less fibrosis (FIG. 25B). On PWD 28, the F814 mice showed significantly more regenerated hair follicles and longer hair fiber length than WT wounds (FIG. 25B), which reflects that the mutant wounds not only healed with a better outcome but also healed faster. Nevertheless, it is still uncertain which cell types are affected by this mutation. However, the data shows that in the periarticular subcutaneous adipose fibroblasts, F814 mouse cells are resistant to OSM-induced collagen (COL1/3) deposition unlike the WT as demonstrated by Picrosirius Red staining, which supports the in vivo data (FIG. 28).

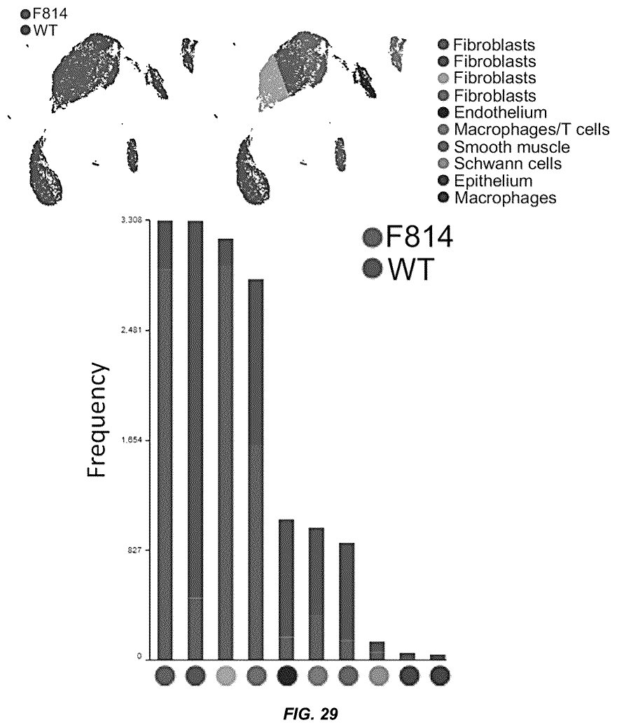

To interrogate the cellular and molecular basis for the increased WIHN observed in F814 wounds, single-cell RNA sequencing (scRNA-seq) was performed on PWD14 wounds, which is an active phase for wound healing. Live, Ter119-cells (Ter119 marks erythroid lineage) from 2-3 wounds from each genotype were sorted and processed using 10× genomics technology; cell numbers were downsampled to normalize the number of cells analyzed. UMAP followed by k-means clustering of the data delineated 9 different clusters, 4 of which expressed fibroblast genes and 2 of which were comprised of hematopoietic cells (FIG. 29). Three of the fibroblast clusters and both hematopoietic clusters were disproportionately comprised of cells from one genotype (FIG. 29). As macrophages have been implicated in wound healing and regenerative responses, the two hematopoietic clusters were “reclustered” following exclusion of CD3+ T cells and conducted k-means clustering (FIG. 25C). This analysis yielded eight clusters which were annotated based on their biomarkers (FIG. 25C). Of note, several clusters enriched for pro-inflammatory cytokines and proteins were disproportionately populated by cells derived from WT wounds. In contrast, a cluster evidencing high levels of Ear2, Retnla and Tgfb1, genes associated with anti-inflammatory, pro-regenerative macrophages, was constituted almost exclusively by cells isolated from F814 wounds (FIG. 25C). These data indicate that in addition to fewer macrophages being present in F814 wounds, they are biased toward a less inflammatory phenotype. Re-clustering and k-means clustering of the fibroblasts from both wound types yielded two clusters, with each populated primarily by one genotype (FIG. 25D). In the cluster dominated by cells from F814 wounds, cells expressed genes associated with both papillary (Dpp4/CD26) and reticular (Dlkl) fibroblasts. Notably, this cluster was enriched for genes related to Wnt signaling including Wnt2, Sfrp2 and Sfrp4 (FIG. 25D); Wnt signaling is a known requirement for regenerative fibroblast competency in WIHN. In contrast, the cluster comprised mostly of cells from WT wounds was enriched for genes associated with myofibroblasts including Acta2, Myl9 and Tagln (FIG. 25D). These data suggest that fibroblasts present in F814 wounds have increased competency to support regenerative vs. fibrotic wound resolution. Altogether, these results suggest that the enhanced healing observed in F814 mouse may be a result of macrophage polarization towards an anti-inflammatory phenotype and improvement of the milieu in the wound.

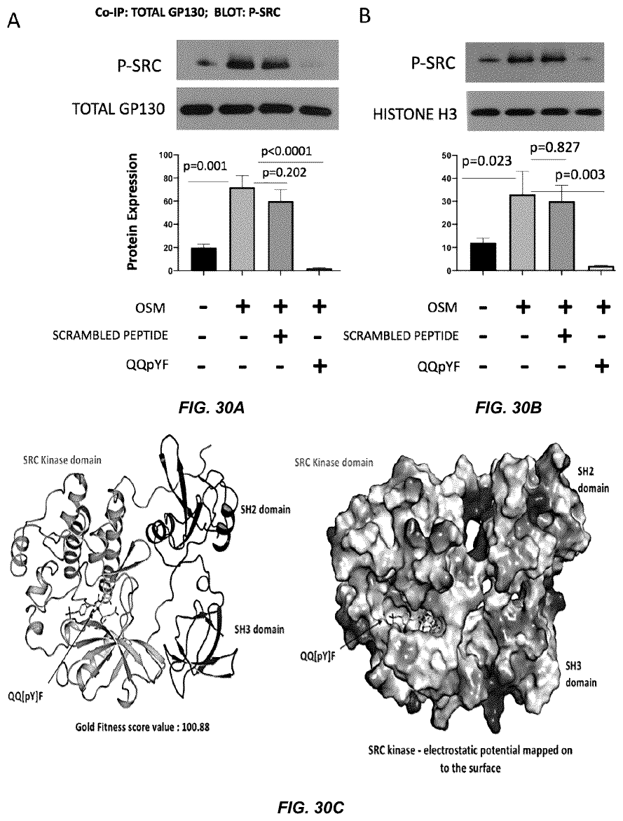

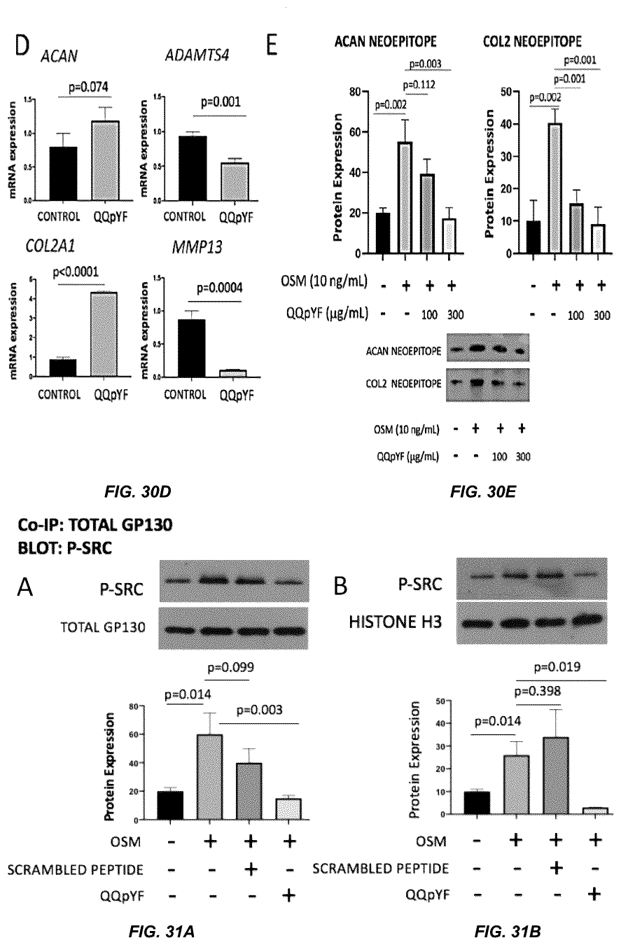

The studies delineate that gp130 Y814 regulates SRC downstream signaling; since SRC is known to associate with gp130, it was hypothesized that gp130 and SRC are capable of physically interacting on Y814 and hindering this interaction will prevent SRC from signaling. In order to impede this interaction, experiments were performed using an innovative peptide library screen with short and overlapping peptides mimicking the motif of gp130 around Y814 that were individually tested for pSRC downregulation; these peptides ranged from 4-15 amino acids. The peptides contained a protein sequence stretch of consecutive amino acids surrounding gp130 Y814 residue. To test their efficacy, pig articular chondrocytes were stimulated with or without OSM in presence or absence of the peptides followed by co-immunoprecipitation to determine interaction and western blot detecting pSRC activity. Out of all the synthesized peptides, a 4-amino acid interfering peptide (QQ[pY]F), proved to be the most efficacious at physically hindering gp130-SRC protein-protein interaction in pig articular chondrocytes by binding to SRC (c-SRC), preventing accessibility to Y814 as an active site (FIG. 30A, FIG. 31A) and decreasing pSRC activation (FIG. 30B, FIG. 31B). Due to sequence specificity, the peptide is capable of discriminating among other gp130 regions serving as a competitive inhibitor. Docking for peptide-protein interaction interfaces confirmed that peptide QQpYF has a strong binding affinity to SRC as shown by the spatial proximity through protein folding in resolved 3D structure (FIG. 30C).

To determine the effect of peptide QQpYF on matrix degeneration, qPCR was performed to quantify gene expression of matrix degrading enzymes in human adult OA articular chondrocytes. Transcription of ADAMTS4/5 and MMP13, were markedly lower in cells treated with peptide QQpYF while biosynthesis of COL2 and aggrecan (ACAN) was increased (FIG. 30D). Further, a neoepitope explant assay was performed where pig articular cartilage explants were stimulated with or without OSM and treated with or without peptide QQpYF at different doses and the levels of cleaved ACAN and COL2 neoepitope were measured. The results confirmed the protective effect of peptide QQpYF on matrix degeneration as shown by the decrease of aggrecanase and collagenase activity (FIG. 30E).

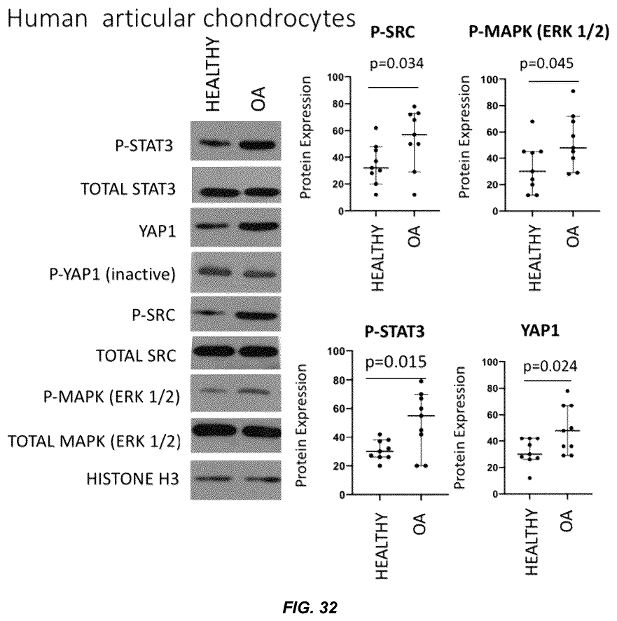

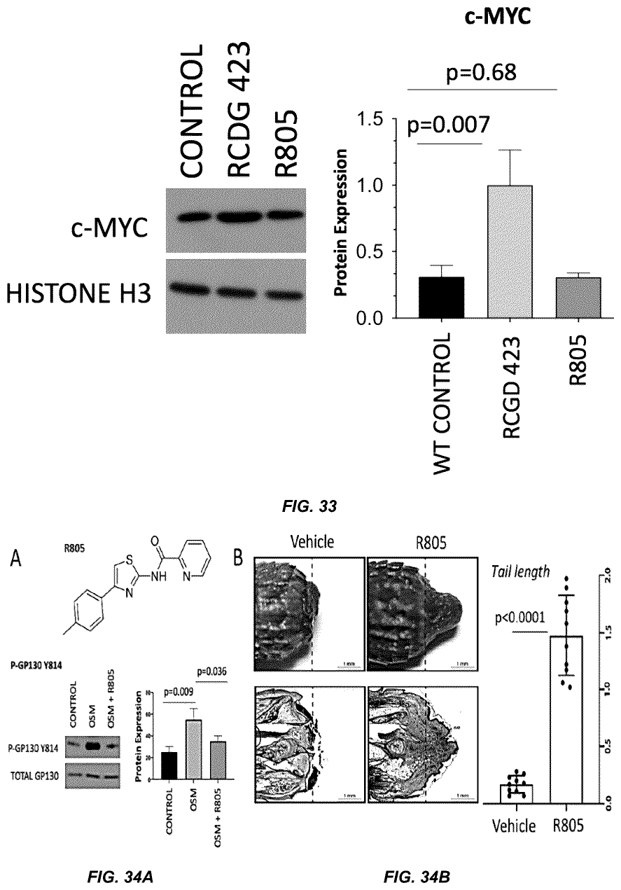

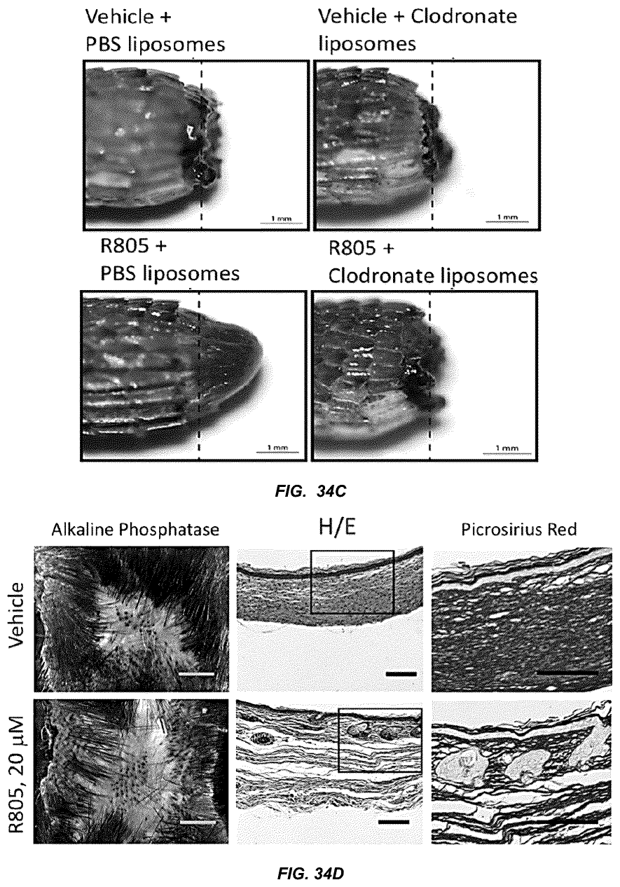

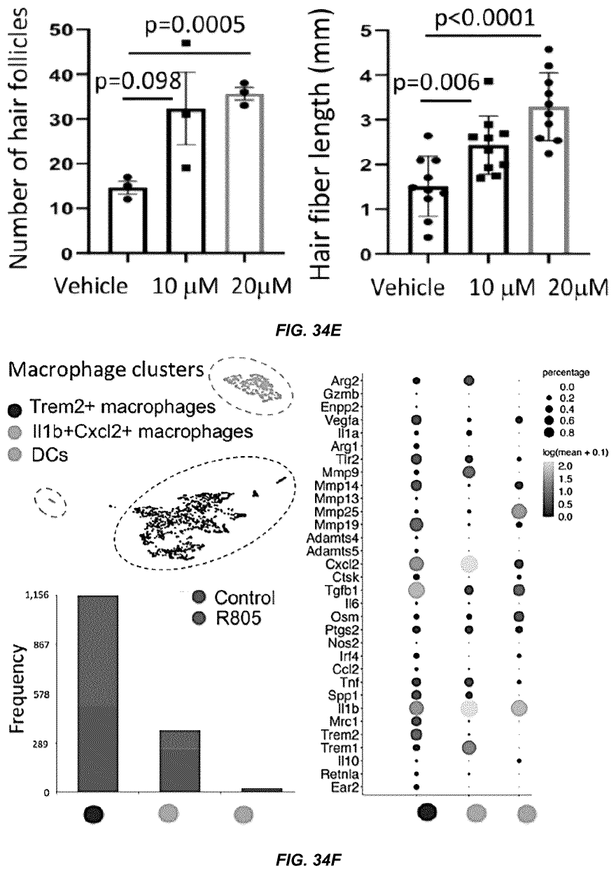

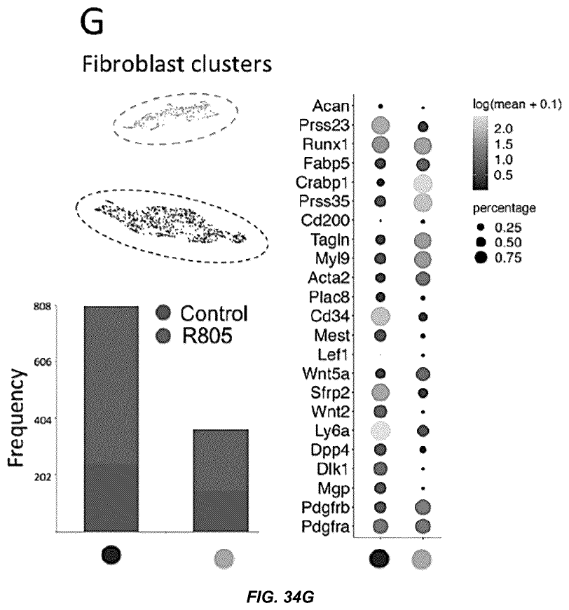

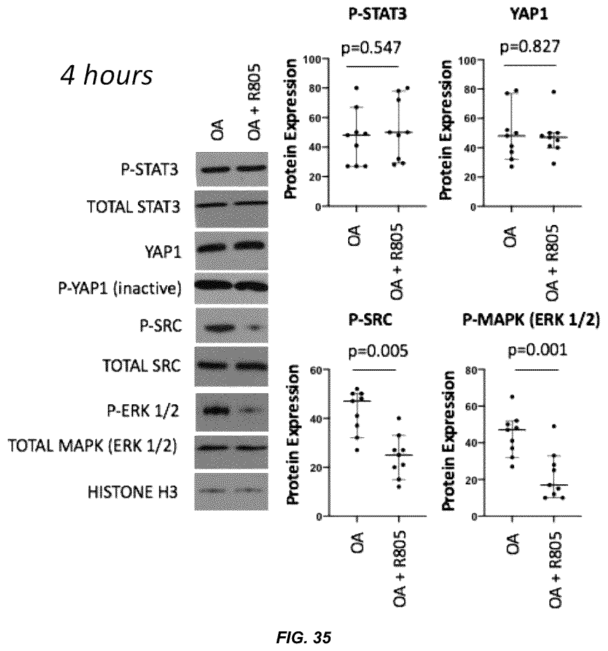

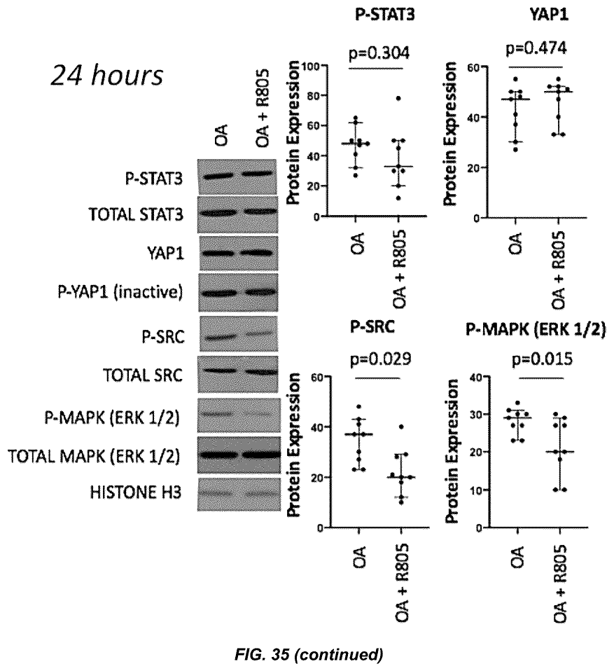

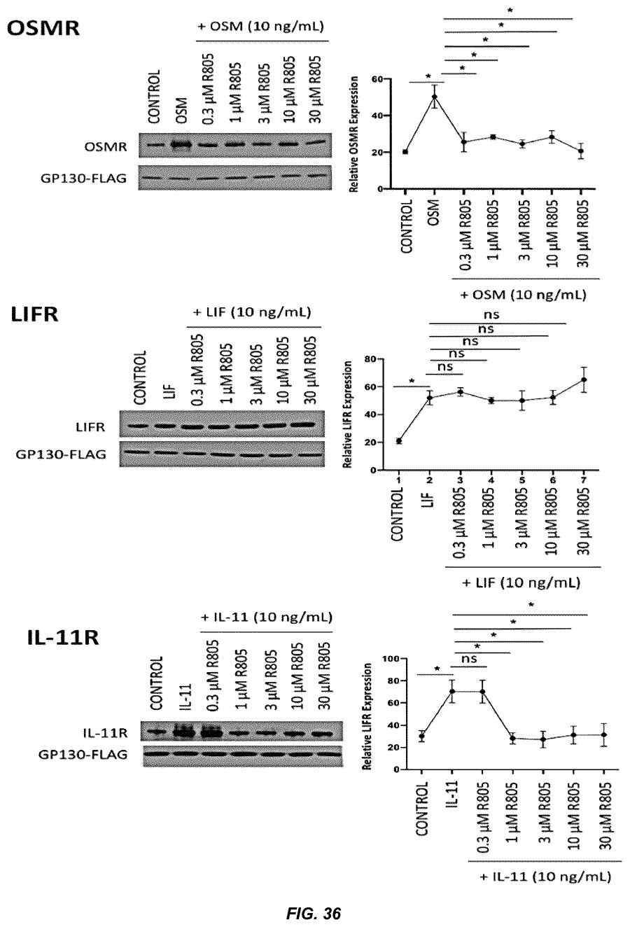

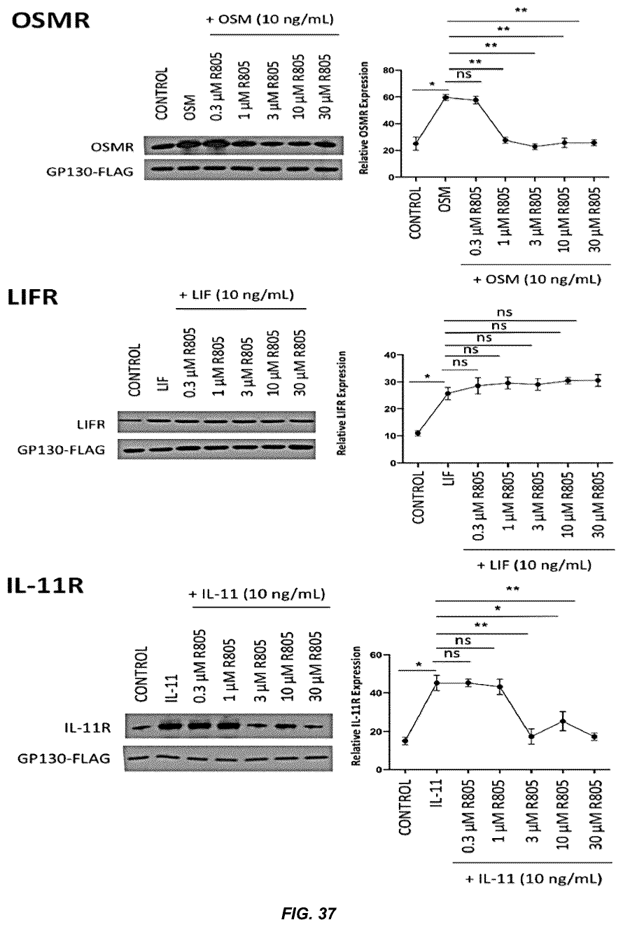

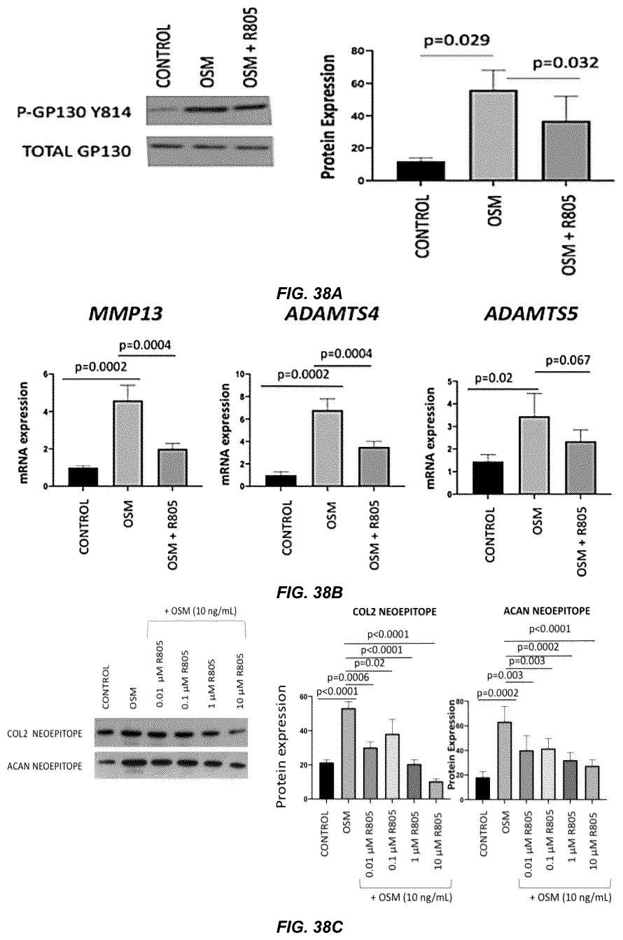

The previous studies in the areas of cartilage tissue degeneration and repair have identified a small molecule, RCGD 423, capable of modulating gp130 receptor signaling. RCGD 423 prevents activation of MAPK/ERK and NF-κB pathways and demonstrates strong anti-inflammatory and anti-degenerative outcomes; this molecule was also shown to highly activate STAT3 signaling and its downstream target, proto-oncogene MYC, deeming activation of this signaling potentially detrimental if hyperactivated, which is unsuitable for therapy. Therefore, it was imperative to find an analog that induces the equivalent beneficial functional outcomes but that does not upregulate MYC signaling while preventing upregulation of pro-inflammatory pathways. The detailed analysis of human primary OA chondrocytes has demonstrated high levels of pSTAT3 (phosphorylated) upregulation compared to adult articular chondrocytes from healthy joints (FIG. 32). STAT3 signaling has been shown to be highly upregulated in rapidly growing, anabolic fetal chondrocytes. Thus, STAT3 upregulation in OA cartilage may be due to an intrinsic attempt for the OA chondrocytes to harness their regenerative potential making additional activation of STAT3 during disease progression, at least in context of the joint, unnecessary. Out of a large library of RCGD 423 analogs that did not upregulate MYC in response to IL-6 family of cytokines (FIG. 33), an analog termed R805 was selected (FIG. 34A) for further characterization as it also minimally affected STAT3 signaling in a diseased joint (FIG. 35). R805 not only markedly suppressed IL-6 family-induced heterodimerization of gp130 with their α-receptors (FIG. 36, 37) similar to RCGD 423 and pro-inflammatory signaling in a diseased joint (FIG. 35), but it also selectively inhibited OSM-stimulated activation of Y814 (FIG. 34A, FIG. 38A). To measure the protective effect of R805 on matrix destruction, pig articular chondrocytes were treated with the drug after stimulation with OSM; based on the data, R805 was able to decrease the MMP13 and ADAMTS4/5 gene expression (FIG. 38B) along with the levels of COL2 and ACAN neoepitopes in OSM-treated explants (FIG. 38C).

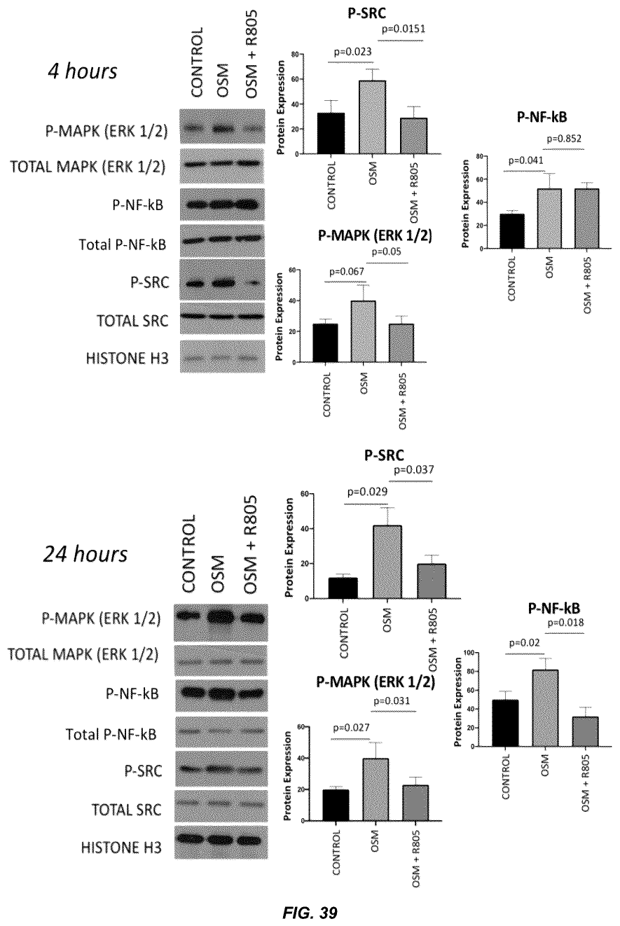

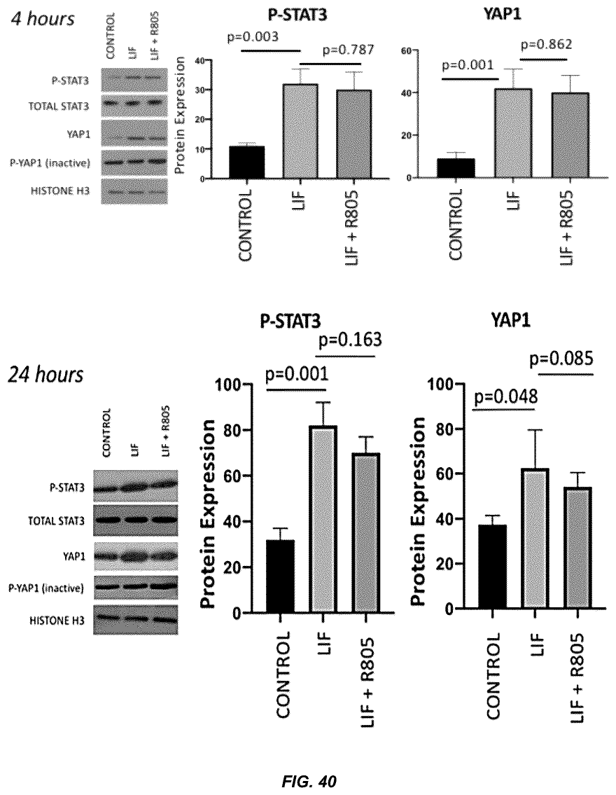

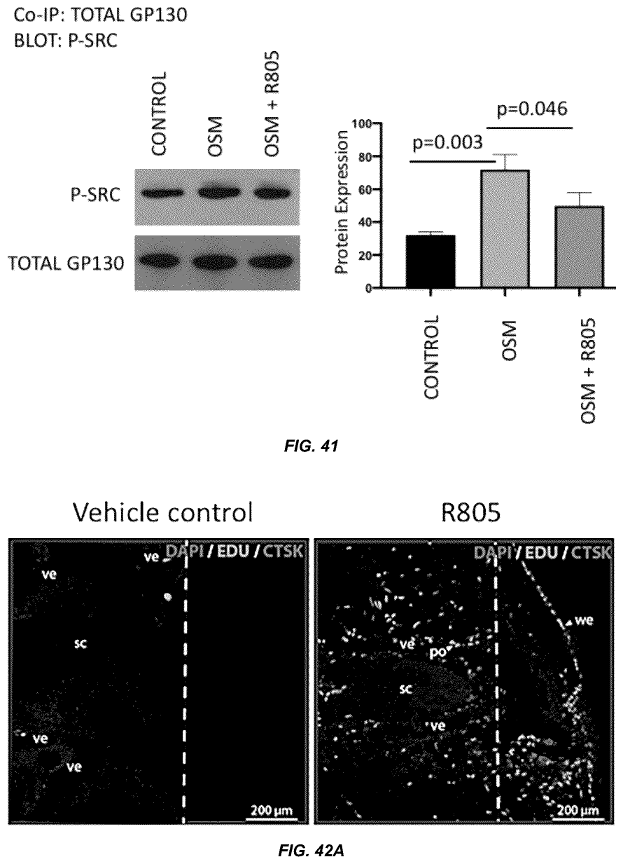

Experiments were performed to determine whether R805 is inclined to shift the signaling outputs downstream of gp130 promoting differential regulation similar to genetic ablation of Y814. As a readout, protein levels of phosphorylated SRC, MAPK (ERK 1/2) and NF-κB were measured in response to OSM while STAT3 and YAP1 levels were evaluated in response to LIF. While pSRC, MAPK (pERK 1/2) and pNF-κB (pNF-κBp65) activation induced by OSM stimulation was markedly suppressed by R805 (FIG. 39), the drug had minimal inhibitory effect on pSTAT3 and YAP1 downstream of LIF (FIG. 40) in pig articular chondrocytes solidifying the role of R805 as a modulator of gp130 receptor signaling rather than a complete inhibitor. Furthermore, R805 was able to prevent endogenous interaction of SRC with gp130 (FIG. 41). These data support the concept that signaling downstream of gp130 can be parsed and modulated such that pro-degenerative activation in a pathological signaling milieu can be greatly reduced while maintaining pro-regenerative signaling supporting the data obtained from WT and F814 mouse cells.

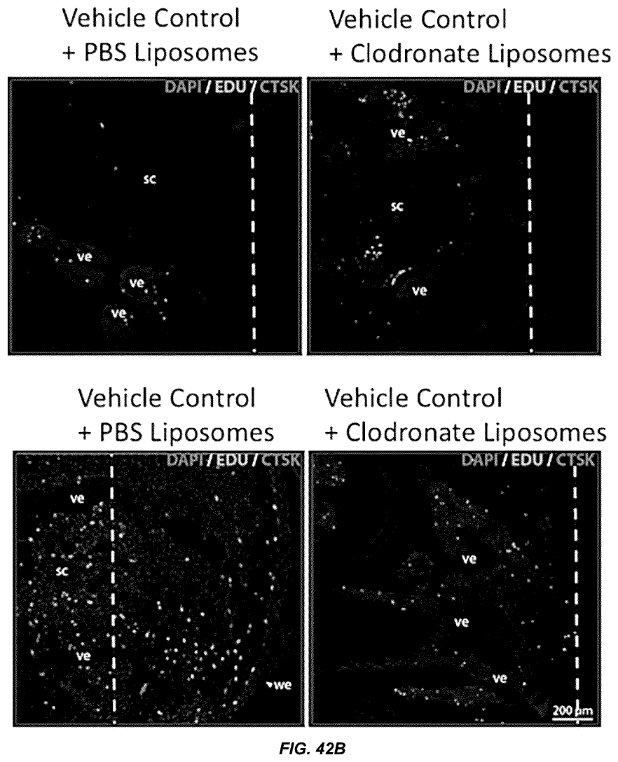

Further experiments were performed to test whether R805 can induce tissue regeneration in multiple animal models. First, a lizard tail injury model was used in a lizard species not naturally capable of tail regeneration, the bearded dragon (Pogona vitticeps). Injection of amputated lizard tails with R805 resulted in tissue regeneration as evidenced by blastema-like structures (FIG. 34B). However, control untreated tails instead formed fibrotic scars with no new tissue besides blood clot accumulation distal to amputation sites and degenerated tissue (FIG. 34B). Masson's trichrome staining showed that unlike R805-treated lizard tails, control tails exhibited open tail wounds at 14 days post-amputation and tail spinal cords formed pericyte scars within vertebrae centers (FIG. 34B). Because lizard tail regeneration is dependent upon the recruitment of CTSK+ macrophage populations to tail amputation sites, control and R805-treated tails were analyzed by CTSK and EdU staining (FIG. 42A). Unlike control, R805-treated lizard tails exhibited CTSK+ macrophage populations that extended into new tissue areas distal to amputation planes and demonstrated significantly higher numbers of proliferating EdU+ wound epithelium and periosteal cells (FIG. 42A). These results suggested that R805 treatment increases macrophage recruitment to lizard tail amputation injuries leading to cell proliferation and tissue regeneration over fibrosis. To further solidify the role of macrophages in inducing lizard tail blastema formation and regeneration, the amputated lizard tails were co-treated with R805 with and without clodronate liposomes, which systemically deplete phagocytic macrophage populations. While lizard tails co-treated with R805 and PBS liposomes exhibited blastema-like structures, co-treatment with clodronate liposomes inhibited R805-induced tissue regrowth (FIG. 34C). Subsequently, CTSK and EdU staining showed that co-treatment with R805 and PBS liposomes exhibited recruitment of CTSK+ macrophages and EdU+ proliferative cells distal to amputation planes while treatment with clodronate liposomes inhibited this macrophage recruitment and cell proliferation (FIG. 42B). These results suggested that macrophage populations are required for R805-induced lizard tail tissue regeneration.

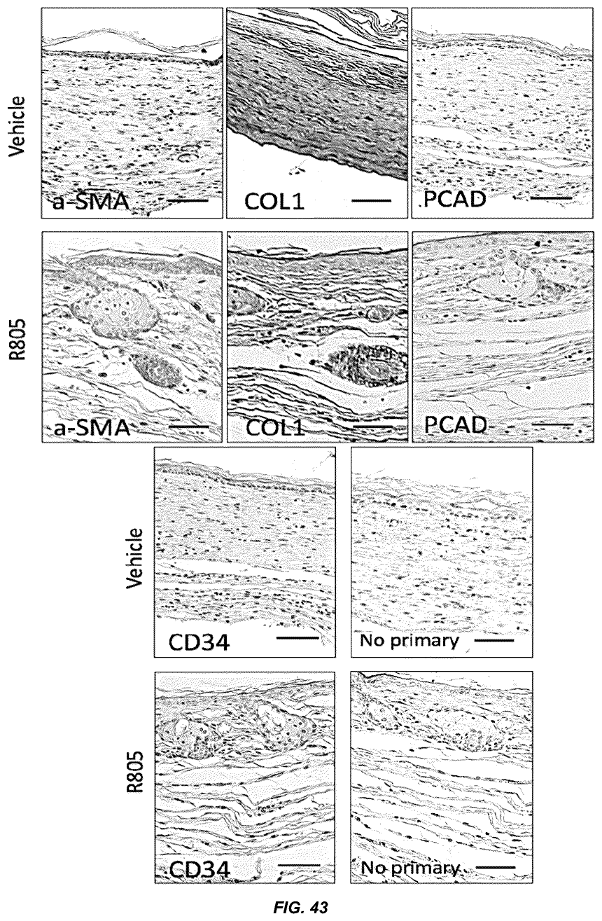

Since the data obtained from lizard tails was encouraging, further experiments were performed to confirm the regeneration potential of R805 in a mammalian model. For this, a skin excisional wound model was employed where the regenerative ability of the R805-treated and untreated mouse WT skin was assessed and compared using the WIHN assay. On PWD 21, the results demonstrated a significant hair follicle neogenesis and dermal regeneration upon topical administration of R805 on the mouse wound (FIG. 34D). The histological observations show that the untreated mouse wound is thinner; furthermore, it positively stained for Picrosirius red, α-SMA and COL1 in the dermis confirming high collagen deposition and is contracted, which indicates fibrosis (FIG. 34D, FIG. 43). This is contrary to R805-treated wound as it is thicker, less contracted and shows less collagen deposition (FIG. 34D, FIG. 43) comparable to the F814 mouse after skin excision. The increase of hair fiber length and number of hair follicles (FIG. 34E) also reveals that the treated wounds healed faster and with a better regenerative outcome, which was also concentration dependent.

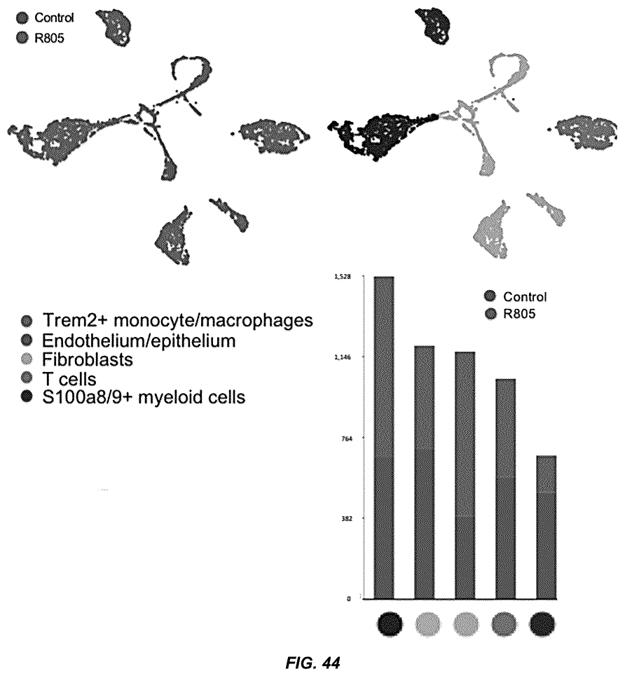

Unsupervised k-means clustering defined a clear macrophage subset (FIG. 44), and re-clustering of these cells delineated two macrophage clusters and a small dendritic cell cluster (FIG. 34F). The larger macrophage cluster contained more cells contributed by R805-treated wounds and evidenced higher expression of genes characteristic of pro-regenerative/anti-inflammatory macrophages including Mrc1, Trem2 and Tgfb1 (FIG. 34F). In contrast, the smaller cluster evidenced more pro-inflammatory gene expression (Il1b, Cxcl2 and Trem1) and was populated primarily by cells from control wounds (FIG. 34F). Re-clustering of fibroblasts in R805-treated and control wounds (FIG. 34G) yielded strikingly similar results to those observed with genetic manipulation of Y814, indicating that pharmacologic modulation of this residue can support tissue regeneration by shifting the microenvironment toward a less pro-inflammatory milieu in the damaged tissue.

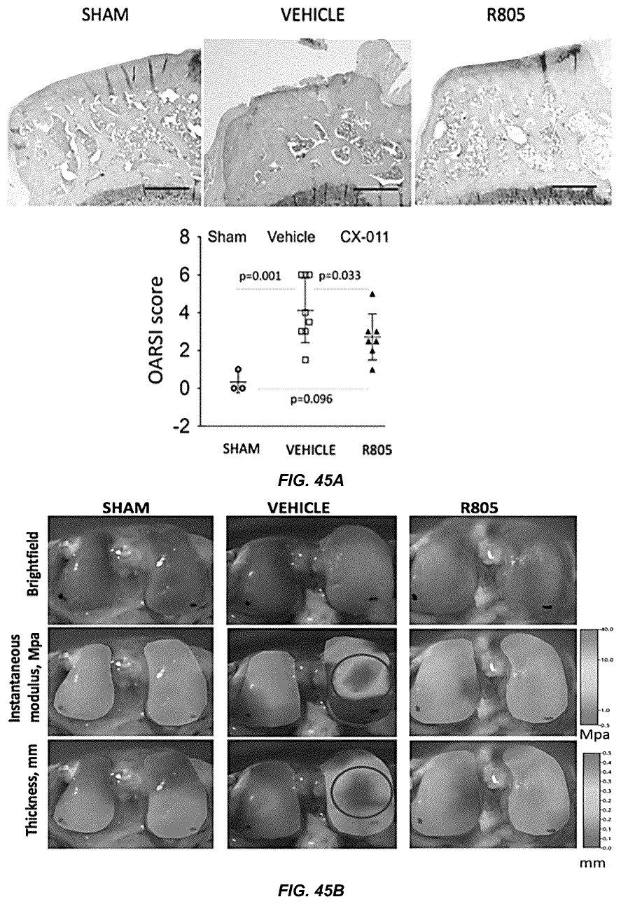

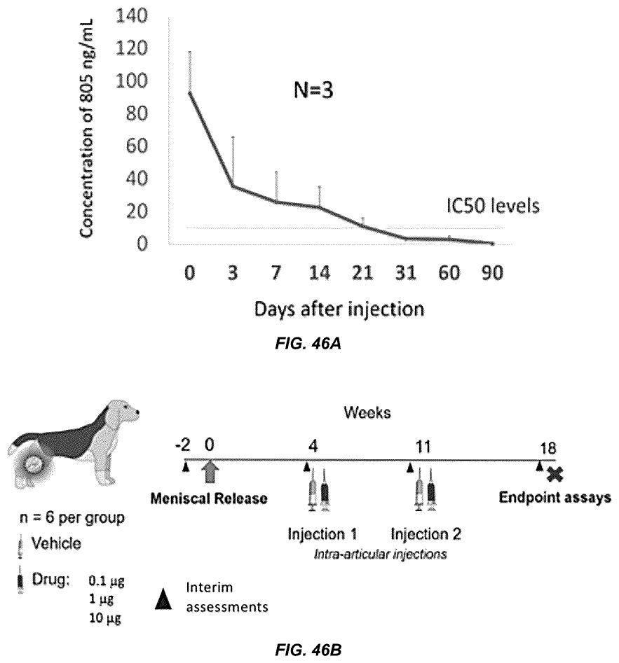

The ability of R805 to improve structural and functional outcomes in vivo was then tested in two commonly used models of posttraumatic OA. First, a rat medial meniscal tear model was used that is useful for the assessment of anti-arthritic drugs in vivo. This study showed prominent protective effects of R805 in the joint (FIG. 45A, B). Since the in vivo results in rats demonstrated efficacy of R805 in alleviating cartilage degradation after injury, a preliminary 90-day pharmacokinetic study was conducted in Beagles that showed a 1 μg intra-articular (IA) injection maintained IC₅₀ concentrations of R805 in the joint for 3 weeks; the compound was never detected in systemic circulation (FIG. 46A). Based on this data, doses of 10 μg, 1 μg and 0.1 μg IA were used in the main experiment. A high-value translational canine OA model employing baseline neuromuscular conditioning and performance assessment on stairs and obstacles, Colorado Pain Scale scoring, gait analysis and CT imaging before conducting a minimally invasive arthroscopic meniscal release was used to evaluate efficacy of R805. The four experimental groups received 1 μg, 0.1 μg or 0.01 μg of R805 or saline on postoperative week 4 and 11 (FIG. 46B).

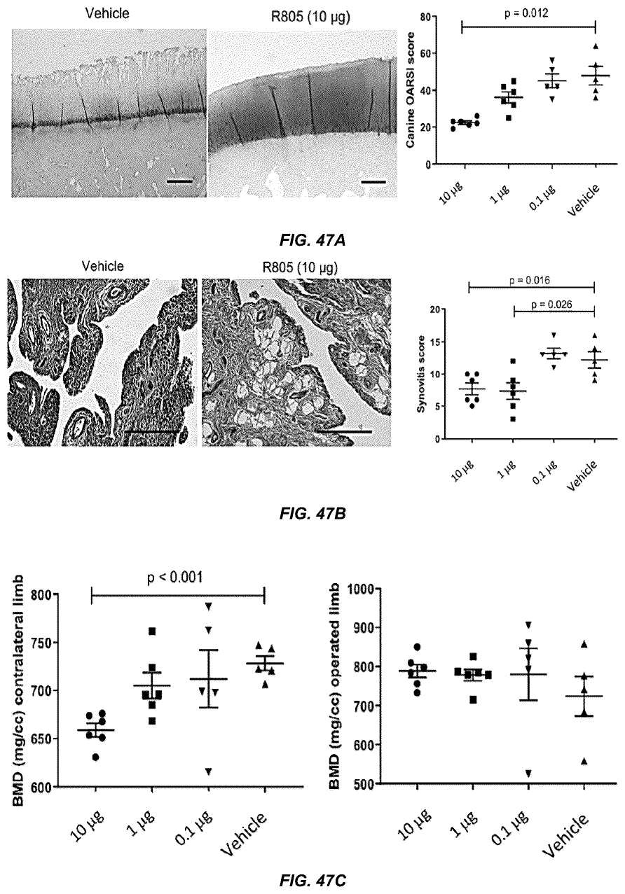

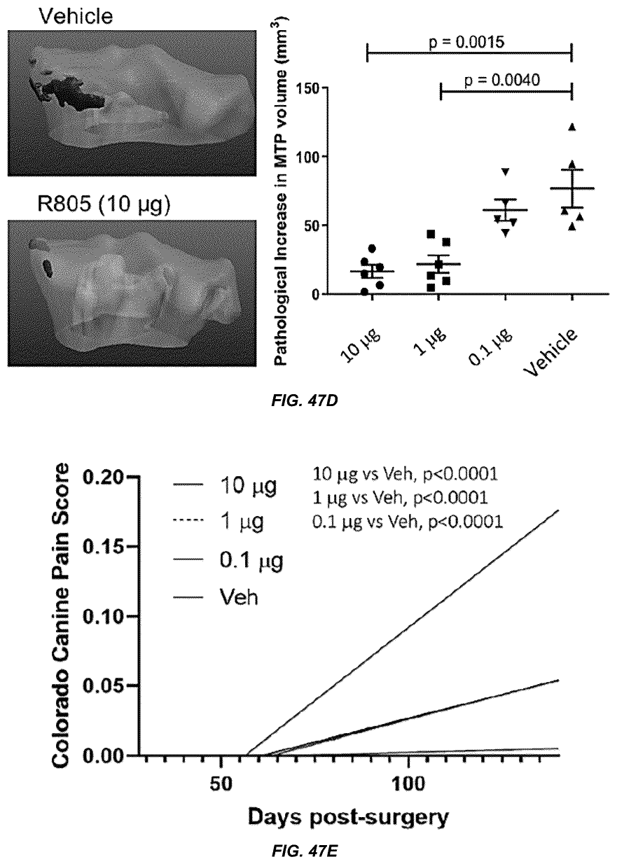

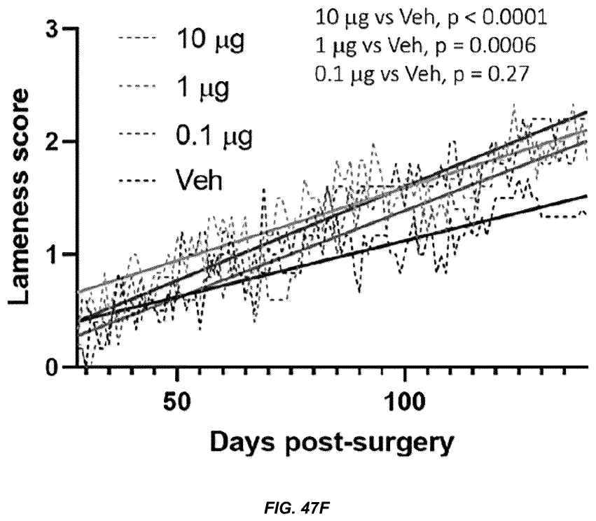

Histological abnormalities due to loss of meniscal load sharing in saline and 0.1 μg dose groups included focal partial- and full thickness cartilage erosions in central non-meniscus covered portion of the tibial plateau and longer linear erosions in the femoral condyle. In adjacent cartilage, there was a loss of the superficial collagen layer and its chondrocytes, loss of proteoglycan staining, invasion of the calcified cartilage by chondroclasts and vasculature, and some thickening (sclerosis) of the subchondral plate. There was incremental improvement in the histological scores as the R805 dose increased; erosions were more shallow and smaller, adjacent cartilage organization was preserved as was proteoglycan staining (FIG. 47A). Synovial membrane in the high and middle dose groups had mild intimal hypertrophy with occasional subintimal mononuclear cell infiltration, whereas low dose and saline treated dogs had a profound increase in subintimal vasculature, new connective tissue and cellular infiltrate comprised of lymphocytes and macrophages (FIG. 47B).

Since these dogs were trained on an agility course and were leash exercised daily outdoors weather permitting, they did not develop hindleg peak vertical force asymmetry as expected. The proportion of forelimb to hindlimb weight bearing in quadrupeds is 60/40. Typically, OA drives bone remodeling and drift in shape in the medial compartment, enlarging the weight bearing surfaces, but almost no change in the cortical outline occurred in the medial tibial plateau in the dogs receiving the 10 ug and 1 ug doses. As expected, bone mineral density increased in the medial tibial plateau in response to meniscal deficiency, but more intriguingly, dogs receiving high dose R805 did not develop subchondral sclerosis in the contralateral knee that usually arises from compensatory load transfer (FIG. 47C). Knee joint shape change, a validated imaging biomarker of OA in people and animals was also clearly controlled in dogs receiving IA R805 (FIG. 47D). This lack of the joint shape in the high dose group was consistent with pain scoring and gait analysis data that show the high dose group had minimal pain (FIG. 5E) and lameness (FIG. 47F).

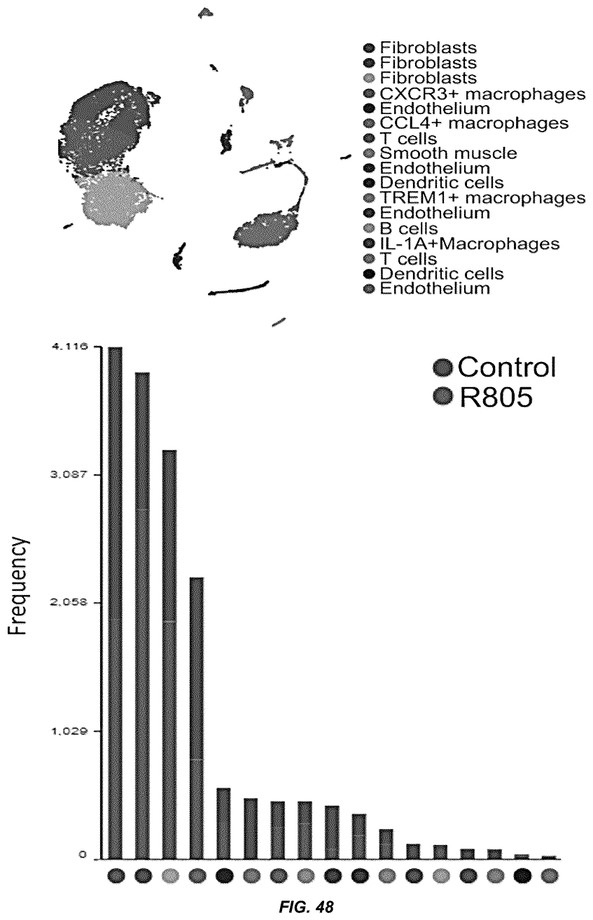

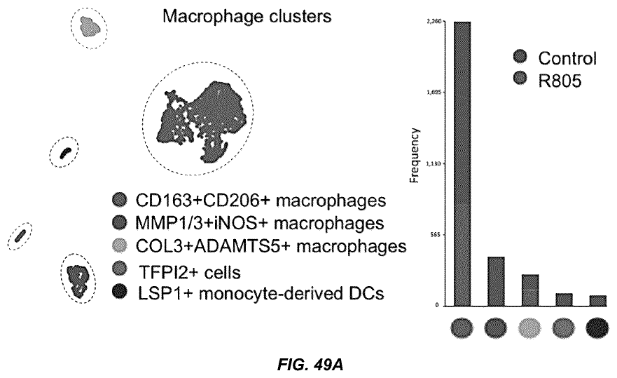

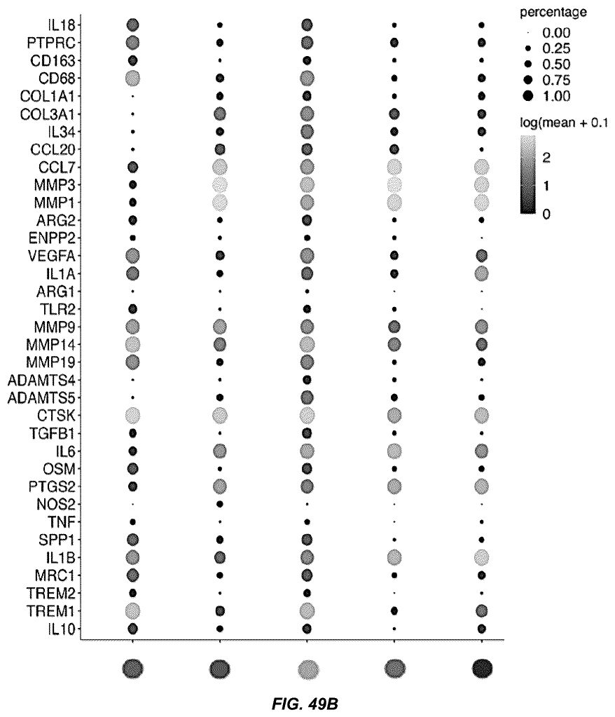

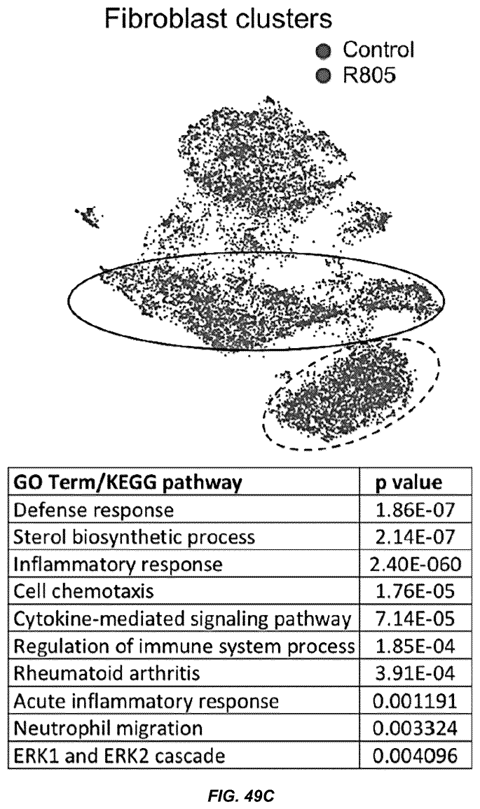

To further confirm the histological findings of reduced cartilage degeneration and synovial fibrosis, scRNA-seq was conducted on synoviocytes isolated from R805-treated and control animals. Unsupervised clustering identified several clusters expressing genes associated with macrophages (FIG. 48); re-clustering of these cells defined 5 clusters (FIG. 49A). The largest cluster was biased toward control cells and was enriched for cells expressing anti-inflammatory macrophage markers including CD206/Mrc1 and CD163 (FIG. 49A). Notably, one cluster populated entirely by cells derived from control animals expressed macrophage markers at lower levels but concurrently expressed high levels of the secreted collagenases MMP1 and MMP3 along with NOS2/Inos (FIG. 49B). Moreover, this population also expressed COL1A1 and COL3A1, suggestive of a potential fibrocyte identity. Re-clustering of synovial fibroblasts defined a clear bias in clustering based on drug treatment (FIG. 49C, ovals). Gene ontology analysis of genes significantly enriched in control vs. R805-treated fibroblasts (FDR<0.05, FC>1.5) revealed significant enrichment of categories related to inflammatory signaling and recruitment of inflammatory cells (FIG. 49C), anchored by enrichment of genes including IL36B, PTGS2 (COX2), IL36G, SAA1, TLR4, CCL3 and IL6. Together, these data show that R805 treatment biases the joint environment away from a chronic inflammatory identity following injury, therefore reducing the progression of cartilage degeneration and synovial fibrosis. Importantly, these findings were consistent with the data previously obtained from F814 mouse skin.

A number of embodiments of the invention have been described. Nevertheless, it will be understood that various modifications may be made without departing from the spirit and scope of the invention. Accordingly, other embodiments are within the scope of the following claims.

1. An isolated peptide of 4-50 amino acids that contains the sequence QQ(pY)F.

2. The isolated peptide of claim 1, wherein the peptide is cyclized with a reagent, thereby forming a cyclic peptide.

3. The isolated peptide of claim 2, wherein the reagent is a thiol-reactive reagent.

4. The isolated peptide of claim 3, wherein the thiol-reactive reagent is selected from the group consisting of: 1,2-bis(bromomethyl)benzene, 1,4-bis(bromomethyl)benzene, 2,6-bis(bromomethyl)pyridine, substituted bis(bromomethyl)benzenes and (E)-1,4-dibromobut-2-ene.

5. The isolated peptide of claim 2, wherein the thiol-reactive reagent is 1,3-bis(bromomethyl)benzene.

6. The isolated peptide of claim 1, wherein the peptide further comprises a cell penetrating peptide (CPP or PTD) sequence.

7. The isolated peptide of claim 1, wherein the peptide comprises one or more non-naturally occurring amino acids.

8. The isolated peptide of claim 1, wherein the peptide comprises one or more D-amino acids.

9. The isolated peptide of claim 1, wherein the peptide comprises a sequence set forth in Table 1 and containing QQ(pY)F.

10. A pharmaceutical composition comprising the peptide of claim 1 and a pharmaceutically acceptable carrier.

11. A pharmaceutical composition of claim 10 further comprising a compound Of

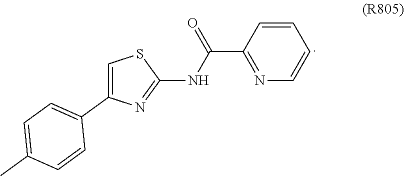 [SMILES: Cc1ccc(-c2csc(NC(=O)c3ccccn3)n2)cc1]

12. A method for the treatment of a gp130 associated disease or disorder in a subject comprising administering to said subject in need thereof of a therapeutically effective amount of a peptide of claim 1.

13. The method of claim 12, wherein the gp130 disease or disorder is an inflammatory disease or disorder or cell proliferative disease or disorder.

14. The method of claim 13, wherein the inflammatory disease or disorder or cell proliferative disease or disorder is selected from the group consisting of stroke, heart disease, cartilage degeneration, hair-loss, wound healing, arthritis, neurodegenerative disorders, aging, psoriasis, rosacea, lupus, rheumatoid arthritis, inflammatory bowel disease, fibrosis and cancer.

15. A method of modulating IL-6 mediated inflammatory cascade in a cell comprising contacting the cell with a peptide of claim 1.

16. The method of claim 15, wherein the cell is a chondrocyte.

17. A method of treating an acute or chronic inflammatory state comprising contact a subject with a peptide of claim 1.

18. (canceled)

19. A method of inhibiting the production or induction of pro-inflammatory genes, cytokines or mediators comprising contacting a cell or subject with a peptide of claim 1.

20. A method of inhibiting the production or induction of extracellular matrix degrading enzymes comprising contacting a cell or subject with a peptide of claim 1.

21. A polyclonal antibody that specifically binds to an epitope comprising or corresponding to pY814 of SEQ ID NO:26.

22-23. (canceled)

24. A non-human transgenic animal expressing a gp130 variant or homolog, wherein the gp130 variant or homolog comprises a substitution corresponding to the tyrosine residue at position 814 of SEQ ID NO:26 with a phenylalanine and wherein the transgenic animal exhibits reduced fibrosis and improved tissue regeneration compared to a wild-type animal.

25. (canceled)

26. The non-human transgenic animal of claim 24, wherein the mouse comprises a gp130 variant having a sequence of SEQ ID NO:27, wherein the gp130 variant comprises an F812 substitution.

27-30. (canceled)


SEQUENCE LISTING (71 sequences)

SEQ ID NO: 1 - protein, 9 aa, Artificial Sequence
  TAT peptide
RKKRRQRRR

SEQ ID NO: 2 - protein, 16 aa, Artificial Sequence
  Penetratin peptide
RQIKIWFQNRRMKWKK

SEQ ID NO: 3 - protein, 21 aa, Artificial Sequence
  Buforin II peptide
TRSSRAGLQFPVGRVHRLLRK

SEQ ID NO: 4 - protein, 26 aa, Artificial Sequence
  Transportan peptide
GWTLNSAGYLLGKINKALAALAKKIL

SEQ ID NO: 5 - protein, 18 aa, Artificial Sequence
  MAP peptide
KLALKLALKALKAALKLA

SEQ ID NO: 6 - protein, 16 aa, Artificial Sequence
  K-FGF peptide
AAVALLPAVLLALLAP

SEQ ID NO: 7 - protein, 10 aa, Artificial Sequence
  Ku70 peptide
VPMLKPMLKE

SEQ ID NO: 8 - protein, 28 aa, Artificial Sequence
  Prion peptide
MANLGYWLLALFVTMWTDVGLCKKRPKP

SEQ ID NO: 9 - protein, 18 aa, Artificial Sequence
  pVEC peptide
LLIILRRRIRKQAHAHSK

SEQ ID NO: 10 - protein, 21 aa, Artificial Sequence
  Pep-1 peptide
KETWWETWWTEWSQPKKKRKV

SEQ ID NO: 11 - protein, 18 aa, Artificial Sequence
  SynB1 peptide
RGGRLSYSRRRFSTSTGR

SEQ ID NO: 12 - protein, 15 aa, Artificial Sequence
  Pep-7 peptide
SDLWEMMMVSLACQY

SEQ ID NO: 13 - protein, 12 aa, Artificial Sequence
  HN-1 peptide
TSPLNIHNGQKL

SEQ ID NO: 14 - protein, 8 aa, Artificial Sequence
  PTD Consensus sequence
  MISC_FEATURE (1)..(1): Xaa is any basic amino acid
  MISC_FEATURE (2)..(4): Xaa is an alpha-helix enhancing amino acid
  MISC_FEATURE (5)..(5): Xaa is a basic amino acid
  MISC_FEATURE (6)..(7): Xaa is an alpha-helix enhancing amino acid
  MISC_FEATURE (8)..(8): Xaa is a basic amino acid
XXXXXXXX

SEQ ID NO: 15 - protein, 8 aa, Artificial Sequence
  PTD consensus sequence
  MISC_FEATURE (1)..(1): Xaa is any basic amino acid
  MISC_FEATURE (2)..(3): Xaa is any alpha helix enhancing amino acid
  MISC_FEATURE (4)..(5): Xaa is any basic amino acid
  MISC_FEATURE (6)..(7): Xaa is any alpha helix enhancing amino acid
  MISC_FEATURE (8)..(8): Xaa is any basic amino acid
XXXXXXXX

SEQ ID NO: 16 - protein, 7 aa, Artificial Sequence
  PTD sequence
KKRPKPG

SEQ ID NO: 17 - protein, 11 aa, Artificial Sequence
  PTD Consensus sequence
  MISC_FEATURE (1)..(2): Xaa is any alpha helical enhancing amino acid
  MISC_FEATURE (4)..(4): Xaa is any alpha helical enhancing amino acid
  MISC_FEATURE (5)..(5): Xaa is any alpha helical enhancing amino acid or a proline
  MISC_FEATURE (6)..(6): Xaa is any alpha helical enhancing amino acid or basic amino acid
  MISC_FEATURE (7)..(7): Xaa is any basic amino acid
  MISC_FEATURE (8)..(8): Xaa is any alpha helical enhancing amino acid or a proline
  MISC_FEATURE (9)..(9): Xaa is any alpha helical enhancing amino acid
  MISC_FEATURE (10)..(10): Xaa is any basic amino acid
  MISC_FEATURE (11)..(11): Xaa is any alpha helical enhancing amino acid or basic amino acid
XXRXXXXXXXX

SEQ ID NO: 18 - protein, 5 aa, Artificial Sequence
  PTD Consensus sequence
  MISC_FEATURE (2)..(2): Xaa is R or K
  MISC_FEATURE (4)..(4): Xaa is any amino acid
  MISC_FEATURE (5)..(5): Xaa is R or K
KXRXX

SEQ ID NO: 19 - protein, 5 aa, Artificial Sequence
  Peptide Linker
GGGGS

SEQ ID NO: 20 - protein, 5 aa, Artificial Sequence
  Peptide Linker
GGGGS

SEQ ID NO: 21 - protein, 12 aa, Artificial Sequence
  Peptide Linker
GKSSGSGSESKS

SEQ ID NO: 22 - protein, 14 aa, Artificial Sequence
  Peptide Linker
GSTSGSGKSSEGKG

SEQ ID NO: 23 - protein, 18 aa, Artificial Sequence
  Peptide Linker
GSTSGSGKSSEGSGSTKG

SEQ ID NO: 24 - protein, 18 aa, Artificial Sequence
  Peptide Linker
GSTSGSGKPGSGEGSTKG

SEQ ID NO: 25 - protein, 14 aa, Artificial Sequence
  Peptide Linker
EGKSSGSGSESKEF

SEQ ID NO: 26 - protein, 917 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 27 - protein, 917 aa, Mus Musculus
MSAPRIWLAQALLFFLTTESIGQLLEPCGYIYPEFPVVQRGSNFTAICVLKEACLQHYYVNASYIVWKTNHAAVPREQVTVINRTTSSVTFTDVVLPSVQLTCNILSFGQIEQNVYGVTMLSGFPPDKPTNLTCIVNEGKNMLCQWDPGRETYLETNYTLKSEWATEKFPDCQSKHGTSCMVSYMPTYYVNIEVWVEAENALGKVSSESINFDPVDKVKPTPPYNLSVTNSEELSSILKLSWVSSGLGGLLDLKSDIQYRTKDASTWIQVPLEDTMSPRTSFTVQDLKPFTEYVFRIRSIKDSGKGYWSDWSEEASGTTYEDRPSRPPSFWYKTNPSHGQEYRSVRLIWKALPLSEANGKILDYEVILTQSKSVSQTYTVTGTELTVNLTNDRYVASLAARNKVGKSAAAVLTIPSPHVTAAYSVVNLKAFPKDNLLWVEWTPPPKPVSKYILEWCVLSENAPCVEDWQQEDATVNRTHLRGRLLESKCYQITVTPVFATGPGGSESLKAYLKQAAPARGPTVRTKKVGKNEAVLAWDQIPVDDQNGFIRNYSISYRTSVGKEMVVHVDSSHTEYTLSSLSSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANNKKPCPDDLKSVDLFKKEKVSTEGHSSGIGGSSCMSSSRPSISSNEENESAQSTASTVQYSTVVHSGYRHQVPSVQVFSRSESTQPLLDSEERPEDLQLVDSVDGGDEILPRQPYFKQNCSQPEACPEISHFERSNQVLSGNEEDFVRLKQQQVSDHISQPYGSEQRRLFQEGSTADALGTGADGQMERFESVGMETTIDEEIPKSYLPQTVRQGGYMPQ

SEQ ID NO: 28 - DNA, 19 bp, Artificial Sequence
  TBP forward primer
gatggacgttcggtttagg

SEQ ID NO: 29 - DNA, 19 bp, Artificial Sequence
  TBP reverse primer
agcagcacagtacgagcaa

SEQ ID NO: 30 - DNA, 21 bp, Artificial Sequence
  MMP13 forward primer
actgagaggctccgagaaatg

SEQ ID NO: 31 - DNA, 19 bp, Artificial Sequence
  MMP13 reverse primer
gaaccccgcatcttggctt

SEQ ID NO: 32 - DNA, 23 bp, Artificial Sequence
  ADAMTS5 forward primer
gaacatcgaccaactctactccg

SEQ ID NO: 33 - DNA, 20 bp, Artificial Sequence
  ADAMTS5 reverse primer
caatgcccaccgaaccatct

SEQ ID NO: 34 - DNA, 20 bp, Artificial Sequence
  ADAMTS4 forward primer
gcaacgtcaaggctcctctt

SEQ ID NO: 35 - DNA, 25 bp, Artificial Sequence
  ADAMTS4 reverse primer
ctccacaaatctactcagtgaagca

SEQ ID NO: 36 - DNA, 22 bp, Artificial Sequence
  Collagen II forward primer
cctggcaaagatggtgagacag

SEQ ID NO: 37 - DNA, 22 bp, Artificial Sequence
  Collagen II reverse primer
cctggttttccaccttcacctg

SEQ ID NO: 38 - DNA, 19 bp, Artificial Sequence
  Aggrecan forward primer
aggcagcgtgatccttacc

SEQ ID NO: 39 - DNA, 21 bp, Artificial Sequence
  Aggrecan reverse primer
ggcctctccagtctcattctc

SEQ ID NO: 40 - DNA, 36 bp, Artificial Sequence
  Synthetic oligonucleotide
gatggtattttgcccaggagtcagcatgaatccagt

SEQ ID NO: 41 - protein, 15 aa, Artificial Sequence
  Synthetic peptide
RQQYFKQNCSQHESS

SEQ ID NO: 42 - protein, 15 aa, Artificial Sequence
  Synthetic peptide
DGILPRQQYFKQNCS

SEQ ID NO: 43 - protein, 16 aa, Artificial Sequence
  Synthetic peptide
YFKQNCSQHESSPDIS

SEQ ID NO: 44 - protein, 14 aa, Artificial Sequence
  Synthetic peptide
GDGILPRQQYFKQN

SEQ ID NO: 45 - protein, 14 aa, Artificial Sequence
  Synthetic peptide
QYFKQNCSQHESSP

SEQ ID NO: 46 - protein, 15 aa, Artificial Sequence
  Synthetic peptide
VDGGDGILPRQQYFK

SEQ ID NO: 47 - protein, 14 aa, Artificial Sequence
  Synthetic peptide
DGGDGILPRQQYFK

SEQ ID NO: 48 - protein, 15 aa, Artificial Sequence
  Synthetic peptide
QYFKQNCSQHESSPD

SEQ ID NO: 49 - protein, 16 aa, Artificial Sequence
  Synthetic peptide
DGGDGILPRQQYFKQN

SEQ ID NO: 50 - protein, 15 aa, Artificial Sequence
  Synthetic peptide
GDGILPRQQYFKQNC

SEQ ID NO: 51 - protein, 15 aa, Artificial Sequence
  Synthetic peptide
GGDGILPRQQYFKQN

SEQ ID NO: 52 - protein, 15 aa, Artificial Sequence
  Synthetic peptide
GILPRQQYFKQNCSQ

SEQ ID NO: 53 - protein, 14 aa, Artificial Sequence
  Synthetic peptide
PRQQYFKQNCSQHE

SEQ ID NO: 54 - protein, 15 aa, Artificial Sequence
  Synthetic peptide
PRQQYFKQNCSQHES

SEQ ID NO: 55 - protein, 12 aa, Artificial Sequence
  Synthetic peptide
QQYFKQNCSQHE

SEQ ID NO: 56 - protein, 5 aa, Artificial Sequence
  Synthetic peptide
QQYFK

SEQ ID NO: 57 - DNA, 23 bp, Artificial Sequence
  Cas9 sgRNA
aaaatgtgaaatctctggacagg

SEQ ID NO: 58 - protein, 10 aa, Artificial Sequence
  Synthetic peptide
  MISC_FEATURE (5)..(5): Y at position 5 is phosphorylated or not phosphorylated
PRQPYFKQNC

SEQ ID NO: 59 - DNA, 50 bp, Artificial Sequence
  Oligonucleotide fragment
atgagatcttgcccaggcaaccgtatttcaagcagaactgcagtcagcct

SEQ ID NO: 60 - DNA, 51 bp, Artificial Sequence
  Oligonucleotide fragment
atgagatcttgcccaggcaaccgtttttcaagcagaactgcagtcagcctg

SEQ ID NO: 61 - protein, 8 aa, Artificial Sequence
  Synthetic peptide
ILPRQQYF

SEQ ID NO: 62 - protein, 8 aa, Artificial Sequence
  Synthetic peptide
QYFKQNCS

SEQ ID NO: 63 - protein, 8 aa, Artificial Sequence
  Synthetic peptide
LPRQQYFK

SEQ ID NO: 64 - protein, 8 aa, Artificial Sequence
  Synthetic peptide
RQQYFKQN

SEQ ID NO: 65 - protein, 8 aa, Artificial Sequence
  Synthetic peptide
PRQQYFKQ

SEQ ID NO: 66 - protein, 15 aa, Artificial Sequence
  Synthetic peptide
HVDGGDGILPRQQYF

SEQ ID NO: 67 - protein, 16 aa, Artificial Sequence
  Synthetic peptide
QYFKQNCSQHEESSPD

SEQ ID NO: 68 - protein, 14 aa, Artificial Sequence
  Synthetic peptide
GILPRQQYFKQNCS

SEQ ID NO: 69 - protein, 8 aa, Artificial Sequence
  Synthetic peptide
GILPRQQY

SEQ ID NO: 70 - protein, 6 aa, Artificial Sequence
  Synthetic peptide
FKQNCS

SEQ ID NO: 71 - protein, 13 aa, Artificial Sequence
  Synthetic peptide
HWWYITTGPVREK